(19) United States
(12) Patent Application Publication
(10) Pub. No.: US 2025/0006331 A1
(43) Pub. Date: Jan. 2, 2025

(54) QUANTITATIVE SYSTEMS PHARMACOLOGY METHODS FOR IDENTIFYING THERAPEUTICS FOR DISEASE STATES

(71) Applicant: UNIVERSITY OF PITTSBURGH-OF THE COMMONWEALTH SYSTEM OF HIGHER EDUCATION, Pittsburgh, PA (US)

(72) Inventors: Douglas Lansing TAYLOR, Pittsburgh, PA (US); Albert H. GOUGH, Glenshaw, PA (US); Andrew Michael STERN, Bradenton, FL (US)

(21) Appl. No.: 18/686,925

(22) PCT Filed: Aug. 31, 2022
(86) PCT No.: PCT/US2022/042153

§ 371 Date: Feb. 27, 2024

Related U.S. Application Data
(60) Provisional application No. 63/238,955, filed on Aug. 31, 2021.
(60) Provisional application No. 63/338,148, filed on May 4, 2022.

(51) Int. Cl. G16H 20/10; A61K 31/167; A61K 31/427; G16B 25/10; G16B 40/20

(52) U.S. Cl. CPC G16H 20/10; A61K 31/167; A61K 31/427; G16B 25/10; G16B 40/20

(57) ABSTRACT

An example method for quantitative systems pharmacology (QSP) includes analyzing, using a computing device, RNA sequencing (RNA-seq) data for a plurality of patients having a disease. The analysis identifies a plurality of differentially expressed genes (DEGs) and a plurality of differentially enriched biological pathways. Additionally, the method includes deriving, using the computing device, a plurality of gene expression signatures associated with each of a plurality of disease states of the disease using the DEGs and the differentially enriched biological pathways. The method also includes identifying, using the computing device, a plurality of drugs predicted to reverse a particular gene expression signature associated with a particular disease state of the disease. The method further includes prioritizing, using the computing device, the drugs predicted to normalize the particular gene expression signature associated with the particular disease state of the disease for further experimental testing.

### CROSS-REFERENCE TO RELATED APPLICATIONS

This application claims the benefit of U.S. provisional patent application No. 63/238,955, filed on Aug. 31, 2021, and titled “QUANTITATIVE SYSTEMS PHARMACOLOGY METHODS FOR IDENTIFYING THERAPEUTICS FOR DISEASE STATES,” and U.S. provisional patent application No. 63/338,148, filed on May 4, 2022, and titled “QUANTITATIVE SYSTEMS PHARMACOLOGY METHODS FOR IDENTIFYING THERAPEUTICS FOR DISEASE STATES,” the disclosures of which are expressly incorporated herein by reference in their entireties.

### STATEMENT REGARDING FEDERALLY FUNDED RESEARCH

This invention was made with government support under Grant nos. DK119973 and DK117881 awarded by the National Institutes of Health. The government has certain rights in the invention.

### BACKGROUND

Complex diseases involve the aberrant expression and function of multiple genes, their products, and signaling pathways often exacerbated by acute and/or chronic environmental cues and an abnormal microbiome. Although each of these dysregulated pathways has the potential to be a drug target, their large number and diversity, the prospect of redundancy, and the uncertainty regarding their individual contribution to pathogenesis especially across a heterogeneous patient population, all present challenges to translating this information into therapeutic strategies. In the case of type 2 diabetes, the disease state appears to result from a few dominant dyshomeostatic pathways that can be pharmacologically modulated to modify the disease. For non alcoholic fatty liver disease (NAFLD) the pathophysiology appears more complicated thwarting successful drug development efforts to date and apparently requiring the identification of drugs targeting the disease state in contrast to just simply a particular pathway.

Nonalcoholic fatty liver disease (NAFLD) is also known as metabolic dysfunction associated fatty liver disease (MAFLD) (Eslam, Sanyal et al. 2020) to better reflect the extensive patient heterogeneity. This heterogeneity appears to be a consequence of the complex pathogenesis of NAFLD involving diverse but convergent signaling cues from the environment, the microbiome, metabolism, comorbidities such as type 2 diabetes, and genetic risk factors (Friedman, Neuschwander-Tetri et al. 2018). The traditional NAFLD definition comprises a spectrum of progressive disease states from simple hepatic steatosis (fatty liver) termed NAFL to a more serious condition, nonalcoholic steatohepatitis (NASH), involving inflammation, hepatocyte damage (i.e., ballooning) and most often pericellular fibrosis (Hardy, Oakley et al. 2016, Sanyal 2019). NASH itself is a risk factor for cirrhosis and end-stage liver disease requiring liver transplantation (Younossi, Marchesini et al. 2019) and for hepatocellular carcinoma (HCC) that insidiously can progress asymptomatically before cirrhosis is diagnosed (Anstee, Reeves et al. 2019). The prevalence of NAFLD is approximately 25% across adult populations world-wide with the proportion of those with NASH predicted to increase over the next decade (Younossi, Marchesini et al. 2019).

Despite the major public health problem NAFLD presents and the economic burden it exacts (Younossi, Henry et al. 2018), no single drug has yet been specifically approved for NAFLD (Polyzos, Kountouras et al. 2018, Sanyal 2019). The challenges facing this unmet need appear to be rooted in the complexity and intrinsic patient heterogeneity of NAFLD. NAFLD has variable rates of progression and clinical manifestations across individual patients (Friedman, Neuschwander-Tetri et al. 2018), with most patients progressing to advanced fibrosis over decades in contrast to approximately 20% who progress much more rapidly (McPherson, Hardy et al. 2015, Singh, Allen et al. 2015).

### SUMMARY

Described herein is a device integrating a clinical data-based computational module, public databases, and a clinically relevant human experimental disease model to 1) derive molecular signatures for a disease state, such as lobular inflammation or primarily fibrosis in non-alcoholic fatty liver disease (NAFLD) that are intrinsic to the disease patholphysiology; 2) predict drugs that can revert these disease state signatures and the biomarkers indicative of the disease state (e.g cytokine release for immune activation and TIMP1 for fibrosis); 3) experimentally test and validate these computational inferences; 4) employ an iterative strategy to optimize therapeutic regimens that include drug combinations; 5) identify disease mechanisms and validate biomarkers; and/or 6) optimize clinical trial design to address patient heterogeneity. When applied to NAFLD, this platform prioritizes drugs having a selective mode-of-action with the potential to induce direct downstream pleiotropic effects that independently have been implicated in disease progression (i.e., normalize the disease state).

An example method for quantitative systems pharmacology (QSP) includes analyzing, using a computing device, RNA sequencing (RNA-seq) data for a plurality of patients having a disease. The analysis identifies a plurality of differentially expressed genes (DEGs) and a plurality of differentially enriched biological pathways. Additionally, the method includes deriving, using the computing device, a plurality of gene expression signatures associated with each of a plurality of disease states of the disease using the DEGs and the differentially enriched biological pathways. The method also includes identifying, using the computing device, a plurality of drugs predicted to reverse a particular gene expression signature associated with a particular disease state of the disease. The method further includes prioritizing, using the computing device, the drugs predicted to normalize the particular gene expression signature and/or physiological characteristic associated with the particular disease state of the disease for further experimental testing.

Additionally, in some implementations, the disease is metabolic dysfunction associated fatty liver disease (MAFLD) or non-alcoholic fatty liver disease (NAFLD).

Alternatively or additionally, the disease states include entirely normal and steatosis, predominantly lobular inflammation, and predominantly fibrosis.

Alternatively or additionally, in some implementations, the method further includes testing, using a microphysiological systems (MPS) platform, a drug or combination of drugs selected from the drugs predicted to normalize the particular gene expression signature and/or physiological characteristic associated with the particular disease state of the disease.

In some implementations, the step of analyzing, using the computing device, the RNA-seq data includes mapping a plurality of gene expression values to a plurality of biological pathway expression profiles; and associating the biological pathway expression profiles with the disease states of the disease. Additionally, the step of mapping the gene expression values to the biological pathway expression profiles includes using a gene set variation analysis (GSVA) algorithm. Additionally, the step of associating the biological pathway expression profiles with the disease states of the disease includes using a clustering algorithm.

In some implementations, the step of identifying, using the computing device, the drugs predicted to reverse the particular gene expression signature and/or physiological characteristic associated with the particular disease state of the disease includes using a connectivity map (CMap).

In some implementations, the step of prioritizing for further experimental testing, using the computing device, the drugs predicted to normalize the particular gene expression signature and/or physiological characteristic associated with the particular disease state of the disease includes using a signature frequency ranking algorithm. Alternatively, in other implementations, the step of prioritizing for further experimental testing, using the computing device, the drugs predicted to normalize the particular gene expression signature and/or physiological characteristic associated with the particular disease state of the disease includes using a network mapping algorithm. Optionally, the network mapping algorithm considers best scores or percentile scores.

Alternatively or additionally, in some implementations, the method further includes demonstrating, using a microphysiological systems (MPS) platform, that the drugs predicted to normalize the particular gene expression signature and/or physiological characteristic associated with the particular disease state of the disease reverses or halts the progression of the disease.

Alternatively or additionally, in some implementations, the drugs predicted to normalize the particular gene expression signature and/or physiological characteristic associated with the particular disease state of the disease include a modulator directly acting on one or more targets with or without downstream pleiotropic effects to correct the particular disease state of the disease. Optionally, in some implementations, the method further includes testing, using a microphysiological systems (MPS) platform, the modulator directly acting on one or more targets with or without downstream pleiotropic effects to correct the particular disease state of the disease.

Alternatively or additionally, in some implementations, the drugs include a compound defined by Formula I:

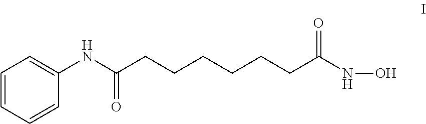 [SMILES: O=C(CCCCCCC(=O)Nc1ccccc1)NO]

or a pharmaceutically acceptable salt thereof.

Alternatively, in some implementations, the drugs further include a compound defined by Formula II:

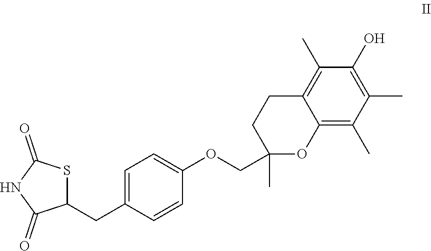 [SMILES: Cc1c(C)c2c(c(C)c1O)CCC(C)(COc1ccc(CC3SC(=O)NC3=O)cc1)O2]

or a pharmaceutically acceptable salt thereof.

Alternatively or additionally, in some implementations, the drugs are selected from the group consisting of 7-hydroxystaurosporine, adenosine-phosphate, alfacalcidol cinnarizine, alvespimycin, alvocidib, ambrisentan, amorolfine, at-7519, auranofin, bezafibrate, brequinar, bromocriptine, brompheniramine, capsaicin, cebranopadol, cefotaxime, chlorpromazine, cladribine, curcumin, cytarabine, dasatinib, dexamethasone, dinoprost, dopamine, eltanolone, ephedrine, ethinylestradiol, fenofibrate, fenoprofen, fexaramine, fexofenadine, flucloxacillin, flucytosine, fluocinolone, Fluvastatin, fulvestrant, geldanamycin, gemcitabine, granisetron, gw-9662, hexestrol, indirubin, iohexol, isoprenaline, itraconazole, k-252a, medrysone, melphalan, mestranol, methylene-blue, mevastatin, midazolam, mifepristone, nifedipine, nitrendipine, norethindrone, Olaparib, olomoucine, oxacillin, oxandrolone, Palbociclib, pd-0325901, phenacetin, piceatannol, probenecid, proxyphylline, PX-12, pyrazolanthrone, ramipril, resveratrol, sildenafil, SN-38, streptozotocin, sulfanitran, tamoxifen, telmisartan, teniposide, tetracycline, thalidomide, trichostatin-a, troxerutin. Vemurafenib, vorinostat, wortmannin, derivatives thereof, and combinations thereof.

Alternatively or additionally, in some implementations, the drugs are selected from the group consisting of 7-hydroxystaurosporine, adenosine-phosphate, alfacalcidol cinnarizine, alvespimycin, alvocidib, amorolfine, at-7519, auranofin, bezafibrate, brequinar, bromocriptine, brompheniramine, capsaicin, cebranopadol, cefotaxime, chlorpromazine, cladribine, curcumin, dasatinib, dexamethasone, dinoprost, dopamine, eltanolone, fenofibrate, fenoprofen, fexaramine, fexofenadine, flucloxacillin, fulvestrant, geldanamycin, gemcitabine, granisetron, gw-9662, hexestrol, iohexol, isoprenaline, itraconazole, k-252a, medrysone, melphalan, mestranol, methylene-blue, midazolam, nitrendipine, norethindrone, Olaparib, olomoucine, oxacillin, oxandrolone, Palbociclib, pd-0325901, phenacetin, piceatannol, probenecid, proxyphylline, PX-12, pyrazolanthrone, ramipril, resveratrol, sildenafil, SN-38, streptozotocin, sulfanitran, tamoxifen, telmisartan, teniposide, tetracycline, thalidomide, trichostatin-a, troxerutin, vorinostat, wortmannin, derivatives thereof, and combinations thereof.

Alternatively or additionally, in some implementations, the drugs are selected from the group consisting of vorinostat, SN-38, auranofin, PX-12, methylene-blue, teniposide, trichostatin-a, trichostatin-a, dexamethasone, geldanamycin, capsaicin, curcumin, itraconazole, midazolam, Olaparib, chlorpromazine, fulvestrant, gemcitabine, alvocidib, brompheniramine, cladribine, dasatinib, dinoprost, fexaramine, fexofenadine, derivatives thereof, and combinations thereof.

Alternatively or additionally, in some implementations, the drugs are selected from the group consisting of eltanolone, fenoprofen, oxandrolone, cefotaxime, amorolfine, dexamethasone, proxyphylline, sn-38, sulfanitran, tetracycline, 7-hydroxystaurosporine, dopamine, medrysone, mestranol, norethindrone, troxerutin, brequinar, bromocriptine, cebranopadol, flucloxacillin, granisetron, hexestrol, iohexol, melphalan, oxacillin, derivatives thereof, and combinations thereof.

Alternatively or additionally, in some implementations, the drugs are selected from the group consisting of bezafibrate, geldanamycin, wortmannin, pd-0325901, piceatannol, fenofibrate, gw-9662, Palbociclib, alvespimycin, olomoucine, dasatinib, telmisartan, pyrazolanthrone, thalidomide, at-7519, nitrendipine, resveratrol, alvocidib, curcumin, probenecid, tamoxifen, sildenafil, methylene-blue, phenacetin, ramipril, derivatives thereof, and combinations thereof.

Alternatively or additionally, in some implementations, the drugs are selected from the group consisting of isoprenaline, fenoprofen, streptozotocin, Palbociclib, 7-hydroxystaurosporine, alvespimycin, k-252a, adenosine-phosphate, alfacalcidol, cinnarizine, ambrisentan, hexestrol, nifedipine, mifepristone, Fluvastatin, mevastatin, cytarabine, ephedrine, ethinylestradiol, tetracycline, fluocinolone, indirubin, dopamine, flucytosine, vemurafenib, derivatives thereof, and combinations thereof.

Alternatively or additionally, in some implementations, the drugs are selected from the group consisting of eltanolone, fenoprofen, oxandrolone, cefotaxime, amorolfine, dexamethasone, proxyphylline, sn-38, sulfanitran, tetracycline, 7-hydroxystaurosporine, dopamine, medrysone, mestranol, norethindrone, troxerutin, brequinar, bromocriptine, cebranopadol, flucloxacillin, granisetron, hexestrol, iohexol, melphalan, oxacillin, derivatives thereof, and combinations thereof.

Alternatively or additionally, in some implementations, the method further includes analyzing the drugs predicted to normalize the particular gene expression signature and/or physiological characteristic associated with the particular disease state of the disease to identify a common thread for further experimental testing.

An example method for treating non-alcoholic fatty liver disease (NAFLD) includes administering the drugs identified by the methods described herein to a subject in need thereof in an effective amount to decrease or inhibit the disease.

Alternatively or additionally, in some implementations, the drugs include a compound defined by Formula I:

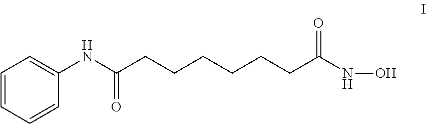 [SMILES: O=C(CCCCCCC(=O)Nc1ccccc1)NO]

or a pharmaceutically acceptable salt thereof.

Alternatively, in some implementations, the drugs further include a compound defined by Formula II:

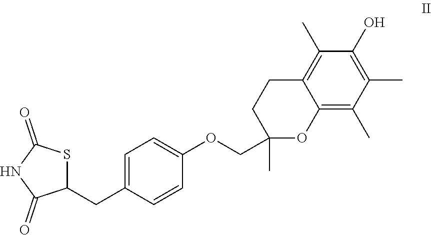 [SMILES: Cc1c(C)c2c(c(C)c1O)CCC(C)(COc1ccc(CC3SC(=O)NC3=O)cc1)O2]

or a pharmaceutically acceptable salt thereof.

Alternatively or additionally, in some implementations, the drugs are selected from the group consisting of 7-hydroxystaurosporine, adenosine-phosphate, alfacalcidol cinnarizine, alvespimycin, alvocidib, ambrisentan, amorolfine, at-7519, auranofin, bezafibrate, brequinar, bromocriptine, brompheniramine, capsaicin, cebranopadol, cefotaxime, chlorpromazine, cladribine, curcumin, cytarabine, dasatinib, dexamethasone, dinoprost, dopamine, eltanolone, ephedrine, ethinylestradiol, fenofibrate, fenoprofen, fexaramine, fexofenadine, flucloxacillin, flucytosine, fluocinolone, Fluvastatin, fulvestrant, geldanamycin, gemcitabine, granisetron, gw-9662, hexestrol, indirubin, iohexol, isoprenaline, itraconazole, k-252a, medrysone, melphalan, mestranol, methylene-blue, mevastatin, midazolam, mifepristone, nifedipine, nitrendipine, norethindrone, Olaparib, olomoucine, oxacillin, oxandrolone, Palbociclib, pd-0325901, phenacetin, piceatannol, probenecid, proxyphylline, PX-12, pyrazolanthrone, ramipril, resveratrol, sildenafil, SN-38, streptozotocin, sulfanitran, tamoxifen, telmisartan, teniposide, tetracycline, thalidomide, trichostatin-a, troxerutin. Vemurafenib, vorinostat, wortmannin, derivatives thereof, and combinations thereof.

Alternatively or additionally, in some implementations, the drugs are selected from the group consisting of 7-hydroxystaurosporine, adenosine-phosphate, alfacalcidol cinnarizine, alvespimycin, alvocidib, amorolfine, at-7519, auranofin, bezafibrate, brequinar, bromocriptine, brompheniramine, capsaicin, cebranopadol, cefotaxime, chlorpromazine, cladribine, curcumin, dasatinib, dexamethasone, dinoprost, dopamine, eltanolone, fenofibrate, fenoprofen, fexaramine, fexofenadine, flucloxacillin, fulvestrant, geldanamycin, gemcitabine, granisetron, gw-9662, hexestrol, iohexol, isoprenaline, itraconazole, k-252a, medrysone, melphalan, mestranol, methylene-blue, midazolam, nitrendipine, norethindrone, Olaparib, olomoucine, oxacillin, oxandrolone, Palbociclib, pd-0325901, phenacetin, piceatannol, probenecid, proxyphylline, PX-12, pyrazolanthrone, ramipril, resveratrol, sildenafil, SN-38, streptozotocin, sulfanitran, tamoxifen, telmisartan, teniposide, tetracycline, thalidomide, trichostatin-a, troxerutin, vorinostat, wortmannin, derivatives thereof, and combinations thereof.

Alternatively or additionally, in some implementations, the drugs are selected from the group consisting of vorinostat, SN-38, auranofin, PX-12, methylene-blue, teniposide, trichostatin-a, trichostatin-a, dexamethasone, geldanamycin, capsaicin, curcumin, itraconazole, midazolam, Olaparib, chlorpromazine, fulvestrant, gemcitabine, alvocidib, brompheniramine, cladribine, dasatinib, dinoprost, fexaramine, fexofenadine, derivatives thereof, and combinations thereof.

Alternatively or additionally, in some implementations, the drugs are selected from the group consisting of eltanolone, fenoprofen, oxandrolone, cefotaxime, amorolfine, dexamethasone, proxyphylline, sn-38, sulfanitran, tetracycline, 7-hydroxystaurosporine, dopamine, medrysone, mestranol, norethindrone, troxerutin, brequinar, bromocriptine, cebranopadol, flucloxacillin, granisetron, hexestrol, iohexol, melphalan, oxacillin, derivatives thereof, and combinations thereof.

Alternatively or additionally, in some implementations, the drugs are selected from the group consisting of bezafibrate, geldanamycin, wortmannin, pd-0325901, piceatannol, fenofibrate, gw-9662, Palbociclib, alvespimycin, olomoucine, dasatinib, telmisartan, pyrazolanthrone, thalidomide, at-7519, nitrendipine, resveratrol, alvocidib, curcumin, probenecid, tamoxifen, sildenafil, methylene-blue, phenacetin, ramipril, derivatives thereof, and combinations thereof.

Alternatively or additionally, in some implementations, the drugs are selected from the group consisting of isoprenaline, fenoprofen, streptozotocin, Palbociclib, 7-hydroxystaurosporine, alvespimycin, k-252a, adenosine-phosphate, alfacalcidol, cinnarizine, ambrisentan, hexestrol, nifedipine, mifepristone, Fluvastatin, mevastatin, cytarabine, ephedrine, ethinylestradiol, tetracycline, fluocinolone, indirubin, dopamine, flucytosine, vemurafenib, derivatives thereof, and combinations thereof.

Alternatively or additionally, in some implementations, the drugs are selected from the group consisting of eltanolone, fenoprofen, oxandrolone, cefotaxime, amorolfine, dexamethasone, proxyphylline, sn-38, sulfanitran, tetracycline, 7-hydroxystaurosporine, dopamine, medrysone, mestranol, norethindrone, troxerutin, brequinar, bromocriptine, cebranopadol, flucloxacillin, granisetron, hexestrol, iohexol, melphalan, oxacillin, derivatives thereof, and combinations thereof.

An example quantitative systems pharmacology (QSP) device is described herein. The device includes at least one processor; and a memory operably coupled to the at least one processor, where the memory has computer-executable instructions stored thereon. The at least one processor is configured to analyze RNA sequencing (RNA-seq) data for a plurality of patients having a disease, where the analysis identifies a plurality of differentially expressed genes (DEGs) and a plurality of differentially enriched biological pathways. The at least one processor is also configured to derive a plurality of gene expression signatures associated with each of a plurality of disease states of the disease using the DEGs and the differentially enriched biological pathways, identify a plurality of drugs predicted to reverse a particular gene expression signature associated with a particular disease state of the disease, and prioritize for further experimental testing the drugs predicted to normalize the particular gene expression signature and/or physiological characteristic associated with the particular disease state of the disease.

In some implementations, the step of analyzing the RNA-seq data includes mapping a plurality of gene expression values to a plurality of biological pathway expression profiles; and associating the biological pathway expression profiles with the disease states of the disease. Additionally, the step of mapping the gene expression values to the biological pathway expression profiles includes using a gene set variation analysis (GSVA) algorithm. Additionally, the step of associating the biological pathway expression profiles with the disease states of the disease includes using a clustering algorithm.

In some implementations, the step of identifying the drugs predicted to reverse the particular gene expression signature and/or physiological characteristic associated with the particular disease state of the disease includes using a connectivity map (CMap).

In some implementations, the step of prioritizing for further experimental testing the drugs predicted to normalize the particular gene expression signature and/or physiological characteristic associated with the particular disease state of the disease includes using a signature frequency ranking algorithm. Alternatively, in other implementations, the step of prioritizing for further experimental testing the drugs predicted to normalize the particular gene expression signature and/or physiological characteristic associated with the particular disease state of the disease includes using a network mapping algorithm. Optionally, the network mapping algorithm considers best scores or percentile scores.

Alternatively or additionally, in some implementations, the at least one processor is further configured to analyze the drugs predicted to normalize the particular gene expression signature and/or physiological characteristic associated with the particular disease state of the disease to identify a common thread for further experimental testing.

An example quantitative systems pharmacology (QSP) system is described herein. The system includes a microphysiological systems (MPS) platform; and a computing device, where the computing device includes at least one processor and a memory operably coupled to the at least one processor, where the memory has computer-executable instructions stored thereon. The at least one processor is configured to analyze RNA sequencing (RNA-seq) data for a plurality of patients having a disease, where the analysis identifies a plurality of differentially expressed genes (DEGs) and a plurality of differentially enriched biological pathways. The at least one processor is also configured to derive a plurality of gene expression signatures associated with each of a plurality of disease states of the disease using the DEGs and the differentially enriched biological pathways, identify a plurality of drugs predicted to reverse a particular gene expression signature associated with a particular disease state of the disease, and prioritize for further experimental testing the drugs predicted to normalize the particular gene expression signature and/or physiological characteristic associated with the particular disease state of the disease.

In some implementations, the step of analyzing the RNA-seq data includes mapping a plurality of gene expression values to a plurality of biological pathway expression profiles; and associating the biological pathway expression profiles with the disease states of the disease. Additionally, the step of mapping the gene expression values to the biological pathway expression profiles includes using a gene set variation analysis (GSVA) algorithm. Additionally, the step of associating the biological pathway expression profiles with the disease states of the disease includes using a clustering algorithm.

In some implementations, the step of identifying the drugs predicted to reverse the particular gene expression signature and/or physiological characteristic associated with the particular disease state of the disease includes using a connectivity map (CMap).

In some implementations, the step of prioritizing for further experimental testing the drugs predicted to normalize the particular gene expression signature and/or physiological characteristic associated with the particular disease state of the disease includes using a signature frequency ranking algorithm. Alternatively, in other implementations, the step of prioritizing for further experimental testing the drugs predicted to normalize the particular gene expression signature and/or physiological characteristic associated with the particular disease state of the disease includes using a network mapping algorithm. Optionally, the network mapping algorithm considers best scores or percentile scores.

Alternatively or additionally, in some implementations, the at least one processor is further configured to analyze the drugs predicted to normalize the particular gene expression signature and/or physiological characteristic associated with the particular disease state of the disease to identify a common thread for further experimental testing.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a workflow associating NAFLD subtypes with gene expression signatures to computationally predict and prioritize drugs for testing in a microphysiological model of disease progression.

FIG. 2 is Table S1. Index of associated tables, figures, data files or notebook analyses for each step in FIG. 1. See Methods and Results for computational and experimental details.

FIG. 3 illustrates unsupervised classification/clustering of patients according to their KEGG pathway enrichment profiles into three predominant NAFLD clusters; normal & simple steatosis (N&S), predominately lobular inflammation (PLI), and predominately Fibrosis (PF).

FIG. 4 is Table S2 of NAFLD patient subtypes within the three clusters defined in FIG. 3 indicating the high degree of concordance of the clusters from FIG. 3 and the clinical diagnoses.

FIGS. 5A-5C illustrate distribution of differentially enriched pathways and their respective KEGG groups and NAFLD categories among the pairwise cluster comparisons defined in FIG. 3. FIG. 5A illustrates the distribution with respect to KEGG Groups. FIG. 5B illustrates the distribution with respect to NAFLD Categories. FIG. 5C illustrates the distribution with respect to KEGG Groups. FIG. 5C illustrates the top 10 differentially enriched pathways for each comparison along with their association (black circles) with NAFLD categories C1-4.

FIG. 6 is an example from Table S3 of NAFLD patient subtypes within the three clusters defined in FIGS. 5A-5C.

FIGS. 7A-71 illustrates how obeticholic acid, Pioglitazone and Troglitazone reduce both lipid accumulation and stellate cell activation in the human MPS (LAMPS model) treated with NAFLD disease media to induce NAFLD. Obeticholic acid and Pioglitazone are positive controls. Troglitazone was predicted. FIGS. 7A-7C illustrate Albumin, Blood Urea Nitrogen, and Lactate Dehydrogenase curves throughout a 10 day time course. FIG. 7D displays representative 20× Day 10 LipidTOX™ (D) images of LAMPS under each treatment condition (i.e., OCA, TGZ, PGZ and vehicle control). FIG. 7E illustrates hepatocellular steatosis in the OCA, TGZ and PGZ treatment groups compared to vehicle control. FIG. 7F displays representative 20× Day 10 α-SMA images of LAMPS under each treatment condition (i.e., OCA, TGZ, PGZ and vehicle control). FIG. 7G illustrates stellate cell activation intensity in the OCA, TGZ and PGZ treatment groups compared to vehicle control. FIG. 7H illustrates secretion of the pro-fibrotic marker Pro-Col 1a1 in the OCA, TGZ and PGZ treatment groups compared to vehicle control. FIG. 7I illustrates secretion of TIMP-1 in the OCA, TGZ and PGZ treatment groups compared to vehicle control.

FIGS. 8A-8J illustrates how the predicted HDAC inhibitor Vorinostat reduces stellate cell activation, the secretion of both pro-fibrotic markers and inflammatory cytokines in LAMPS models treated with NAFLD disease media to induce NAFLD. FIGS. 8A-8C illustrate Albumin, Blood Urea Nitrogen, and Lactate Dehydrogenase curves throughout a 10 day time course. FIG. 8D displays representative 20× Day 10 LipidTOX™ (D) images of LAMPS under each treatment condition (i.e., OCA, TGZ, PGZ and vehicle control). FIG. 8E illustrates hepatocellular steatosis in the OCA, TGZ and PGZ treatment groups compared to vehicle control. FIG. 8F displays representative 20× Day 10 α-SMA images of LAMPS under each treatment condition (i.e., OCA, TGZ, PGZ and vehicle control). FIG. 8G illustrates stellate cell activation intensity in the OCA, TGZ and PGZ treatment groups compared to vehicle control. FIG. 8H illustrates secretion of the pro-fibrotic marker Pro-Col 1a1 in the OCA, TGZ and PGZ treatment groups compared to vehicle control. FIG. 8I illustrates secretion of TIMP-1 in the OCA, TGZ and PGZ treatment groups compared to vehicle control. FIG. 8J illustrates inflammatory cytokine production observed in the vorinostat treatment group.

FIGS. 9A-9J illustrates the ability to use combinations of predicted drugs to reduce steatosis, stellate cell activation as well as secretion of the pro-fibrotic markers and production of inflammatory cytokines in the LAMPS model of NAFLD. FIGS. 9A-9C illustrate Albumin, Blood Urea Nitrogen, and Lactate Dehydrogenase curves throughout a 10 day time course. FIG. 9D displays representative 20× Day 10 LipidTOX™ (D) images of LAMPS under each treatment condition (i.e., OCA, TGZ, PGZ and vehicle control). FIG. 9E illustrates hepatocellular steatosis in the OCA, TGZ and PGZ treatment groups compared to vehicle control. FIG. 9F displays representative 20× Day 10 α-SMA images of LAMPS under each treatment condition (i.e., OCA, TGZ, PGZ and vehicle control). FIG. 9G illustrates stellate cell activation intensity in the OCA, TGZ and PGZ treatment groups compared to vehicle control. FIG. 9H illustrates secretion of the pro-fibrotic marker Pro-Col 1a1 in the OCA, TGZ and PGZ treatment groups compared to vehicle control. FIG. 9I illustrates secretion of TIMP-1 in the OCA, TGZ and PGZ treatment groups compared to vehicle control. FIG. 9J illustrates inflammatory cytokine production observed when pioglitazone and vorinostat are used in combination.

FIG. 10 is Table 1, which illustrates prioritization of CMap-predicted drugs based on occurrence across multiple NAFLD-associated gene signature queries.

FIG. 11 is an example computing device.

FIG. 12 is Table S4. Gene signature index (created using Data file S3).

FIGS. 13A-13C are Table S5, which illustrates the top 20 drug and small molecule perturbagen predictions from CMap analysis for each of the 12 queries. FIG. 13A illustrates the top drugs for PLI vs N&S pairwise comparison. FIG. 13B illustrates the top drugs for PF vs N&S pairwise comparison. FIG. 13C illustrates the top drugs for PF vs PLI pairwise comparison.

FIG. 14 is Table S6, which illustrates the top 20 hubs ranked by degree in the NAFLD subnetwork.

FIG. 15 is Table S7, which illustrates prioritization of CMap-predicted drugs and small-molecule perturbagens based on NAFLD subnetwork proximity.

FIG. 16 is Table S8, which illustrates drug binding and cytotoxicity profiles for compounds used in MPS (LAMPS) studies.

FIG. 17 illustrates using the Biomimetic Human Liver Acinus MicroPhysiology System (LAMPS) for proof-of-concept experimental testing of CMap-predicted drugs.

FIG. 18 illustrates the CMap methodology.

FIG. 19 illustrates NAFLD associated protein interactome.

FIG. 20 is Data file S1, which illustrates DEGs resulting from the 3 pairwise cluster comparisons.

FIG. 21 is Data file S2, which illustrates differentially enriched pathways for each pairwise cluster comparison.

FIG. 22 is Data file S3, which illustrates gene signatures used for CMap analysis.

FIG. 23 is Data file S4, which illustrates non-zero CMAP scores of small molecules with a DrugBank ID for the 12 queries described in FIG. 15 and Methods.

FIG. 24 is Data file S5, which is the list of top 20 CMap (FDR p-value<0.05) predictions from the 12 signatures (196 predictions, 139 unique compounds) using the 2017 LINCS database.

FIG. 25 is Data file S6, which illustrates degree of the nodes in the NAFLD subnetwork.

FIG. 26 is Data file S7, which illustrates network proximity determined Z-scores for the highest ranking CMap-predicted drugs with targets mapping to the NAFLD subnetwork.

FIG. 27 is Table 2, which is cluster based gene signatures queried against the CMap 2017 perturbation database prioritized using the Best Score CMap score. Table 2 prioritizes drugs by the frequency the drugs appear in the gene signature based queries.

FIG. 28 is Table 3, which is cluster based gene signatures queried against the CMap 2020 perturbation database (expanded database) prioritized using the LINCS percentile CMap score. Table 3 prioritizes drugs by the frequency the drugs appear in the gene signature based queries.

FIG. 29 is Table 4, which is network proximity results using the cluster gene signatures queried against the CMap 2017 perturbation database prioritized by best CMap score. Red targets are in the NAFLD subnetwork. Table 4 prioritizes drugs by network proximity.

FIG. 30 is Table 5, which is network proximity results using the cluster gene signatures queried against the CMap 2020 perturbation database (expanded database) prioritized by the LINCS percentile CMap score. Red targets are in the NAFLD subnetwork. Table 5 prioritizes drugs by network proximity.

FIG. 31A illustrates an unbiased machine learning model of patient transcriptomic data identifies and predicts congruent clinical phenotypes within LAMPS. FIG. 31B illustrates the average sensitivity across the bootstrapping instances (numbers in parenthesis are standard deviations) are: 0.66 (0.11), 0.64 (0.12), 0.77 (0.08), 0.93 (0.07); average specificity 0.93 (0.03), 0.83 (0.03), 0.98 (0.02), 0.95 (0.03) for Normal, Steatosis, Lob, and Fibrosis respectively.

FIGS. 32A-32S illustrate control and predicted drugs reduce different NAFLD disease phenotypes in LAMPS models treated with EMS media. FIGS. 32A-32C illustrate Albumin, Blood Urea Nitrogen, and Lactate Dehydrogenase curves throughout a 10 day time course. FIG. 32D displays representative 20× Day 10 LipidTOX™ (D) images of LAMPS under each treatment condition (i.e., OCA, PGZ and vehicle control). FIG. 32E illustrates hepatocellular steatosis in the OCA, PGZ treatment groups compared to vehicle control. FIG. 32F displays representative 20× Day 10 α-SMA images of LAMPS under each treatment condition (i.e., OCA, PGZ and vehicle control). FIG. 32G illustrates stellate cell activation intensity in the OCA, PGZ treatment groups compared to vehicle control. FIG. 32H illustrates secretion of the pro-fibrotic marker Pro-Col 1a1 in the OCA, PGZ treatment groups compared to vehicle control. FIG. 32I illustrates secretion of TIMP-1 in the OCA, PGZ treatment groups compared to vehicle control. FIGS. 32J-32L illustrate Albumin, Blood Urea Nitrogen, and Lactate Dehydrogenase curves throughout a 10 day time course for predicted drug (vorinostat). FIG. 32M displays representative 20× Day 10 LipidTOX™ (FIG. 32D) images of LAMPS under each treatment condition (i.e., vorinostat and vehicle control). FIG. 32N illustrates hepatocellular steatosis in the vorinostat treatment groups compared to vehicle control. FIG. 32O displays representative 20× Day 10 α-SMA images of LAMPS under each treatment condition (i.e., vorinostat and vehicle control). FIG. 32P illustrates stellate cell activation intensity in the vorinostat treatment groups compared to vehicle control. FIG. 32Q illustrates secretion of the pro-fibrotic marker Pro-Col 1a1 in the vorinostat treatment groups compared to vehicle control. FIG. 32Q illustrates secretion of TIMP-1 in the vorinostat treatment groups compared to vehicle control. FIG. 32S illustrates inflammatory cytokine production observed in the vorinostat treatment group.

FIG. 33A-33J illustrate pioglitazone and vorinostat used in combination results in the reduction of steatosis and stellate cell activation as well as the secretion of pro-fibrotic markers and production of inflammatory cytokines in LAMPS models treated with EMS media. FIGS. 33A-33C illustrate Albumin, Blood Urea Nitrogen, and Lactate Dehydrogenase curves throughout a 10 day time course for predicted drug (vorinostat). FIG. 33D displays representative 20× Day 10 LipidTOX™ (FIG. 33D) images of LAMPS under each treatment condition (i.e., combination of pioglitazone and vorinostat and vehicle control). FIG. 33E illustrates hepatocellular steatosis in the combination of pioglitazone and vorinostat treatment groups compared to vehicle control. FIG. 33F displays representative 20× Day 10 α-SMA images of LAMPS under each treatment condition (i.e., combination of pioglitazone and vorinostat and vehicle control). FIG. 33G illustrates stellate cell activation intensity in the combination of pioglitazone and vorinostat treatment groups compared to vehicle control. FIG. 33H illustrates secretion of the pro-fibrotic marker Pro-Col 1a1 in the combination of pioglitazone and vorinostat treatment groups compared to vehicle control. FIG. 333I illustrates secretion of TIMP-1 in the combination of pioglitazone and vorinostat treatment groups compared to vehicle control. FIG. 33J illustrates inflammatory cytokine production observed when pioglitazone and vorinostat are used in combination.

FIG. 34 is Table 6 illustrating the 25 highest ranked CMap-predicted drugs based on frequency of occurrence across multiple NAFLD-associated gene signature queries using the expanded 2020 LINCS database.

FIGS. 35A-35C illustrate distribution of differentially enriched pathways and their respective KEGG groups and NAFLD categories among the pairwise cluster comparisons performed using the patient clinical classifications. FIGS. 35A-35C complement FIGS. 5A-5C. FIG. 35A illustrates the distribution with respect to KEGG Groups. FIG. 35B illustrates the distribution with respect to NAFLD Categories. FIG. 5C illustrates the distribution with respect to KEGG Groups. FIG. 35C illustrates the top 10 differentially enriched pathways for each comparison along with their association (black circles) with NAFLD categories C1-4. FIGS. 35A-35C complement FIGS. 5A-5C.

FIG. 36 are Venn diagrams showing the overlap of differentially enriched pathways (FDR p-value<0.001) identified in the cluster (left circle) and clinical label (right circle) pairwise comparisons. FIG. 36 supports FIGS. 5A-5C and 35A-35C.

FIG. 37 is a concordance analysis of the differentially enriched pathways in the cluster pairwise comparisons (left circle) and pathway list derived from microarray datasets (right circle).

FIG. 38 is a concordance analysis of the differentially enriched pathways in the LAMPS (left circle) and phenotypically matched patient pairwise comparisons.

FIGS. 39A-39C is an Exploratory data analysis and PCA of the patient transcriptome. FIG. 39A shows the boxplots (outliers are not shown) of the log 2 transformed counts per million log 2 (CPM) gene expression values for each patient, ordered by the patient ID (i.e., the order the samples were processed). FIG. 39B is a principal component analysis (PCA) of the log 2 (CPM) gene expression values. FIG. 39C is the PCA plot using the surrogate variable analysis (SVA) corrected gene expression matrix.

### DETAILED DESCRIPTION

Nonalcoholic fatty liver disease (also referred to herein as metabolic dysfunction associated fatty liver disease) is a major public health problem having a complex and heterogeneous pathophysiology involving metabolic dysregulation and aberrant immunologic responses that has made therapeutic development a major challenge. Described herein is an integrated quantitative systems pharmacology (QSP) platform comprised of computational algorithms for predicting drugs for repurposing and/or initiating development of novel therapeutics and a human biomimetic liver acinus microphysiological system (LAMPS) containing four key cell types that recapitulates key aspects of MAFLD progression for candidate drug testing. Analysis of individual patient-derived hepatic RNAseq data encompassing a full spectrum of NAFLD states from simple steatosis, to NASH, advanced fibrosis and cirrhosis, with some type 2 diabetes generated 12 gene signatures associating molecular phenotypes to clinical progression and pathophysiology (e.g., lipotoxicity, insulin resistance, ER and oxidative stress, inflammation, fibrosis). Focusing on normalizing disease states rather than individual genes or pathways in a mechanism-unbiased manner, drugs predicted to invert the expression of NAFLD-associated signatures were identified using connectivity mapping. Two independent approaches for prioritizing predicted drugs identified complementary sets of diverse drugs for testing. In one experiment described herein, vorinostat robustly mitigated inflammation and fibrosis, and significantly reduced disease-associated cell death without an impact on steatosis in the LAMPS experimental model. In contrast, troglitazone reduced steatosis but not fibrosis markers. The QSP platform described herein has predicted many drugs that could be part of a novel drug combination therapeutic strategy for MAFLD.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

“Administration” or “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used herein, the term “comprising” is intended to mean that the systems, compositions and methods include the recited elements, but not excluding others. “Consisting essentially of” when used to define systems, compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps. Embodiments defined by each of these transition terms are within the scope of this invention.

A “control” is an alternative subject or sample used in an experiment for comparison purposes. A control can be “positive” or “negative.”

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “suppress”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

As used herein, by a “subject” is meant an individual. The term “subject” is defined herein to include animals such as mammals, including, but not limited to, primates (e.g., humans), cows, sheep, goats, horses, dogs, cats, rabbits, rats, mice and the like. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. In some embodiments, the subject is a human.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of a disease (e.g., a cancer).

The term “therapeutically effective amount” refers to the amount of the composition used is of sufficient quantity to ameliorate one or more causes or symptoms of a disease or disorder. Such amelioration only requires a reduction or alteration, not necessarily elimination.

As used herein, the term “delivery” encompasses both local and systemic delivery.

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

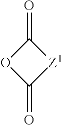

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O—.

A “carbonate ester” group as used herein is represented by the formula Z¹OC(O)OZ².

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

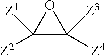

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Example Methods

Example quantitative systems pharmacology (QSP) methods are described herein. This disclosure contemplates that logical operations can be performed using one or more computing devices (e.g., a processing unit and memory as described herein). Additionally, in the examples below, the QSP methods are applied to developing therapeutics for nonalcoholic fatty liver disease (also referred to herein as metabolic dysfunction associated fatty liver disease). It should be understood that NAFLD (or MAFLD) is provided only as an example. This disclosure contemplates that the QSP methods can be applied to developing therapeutics for other diseases including, but not limited to, all solid tumor cancers and diseases where a patient tissue/organ sample can be obtained to perform RNASeq analyses.

As noted above, no single drug has yet been specifically approved for NAFLD (or MAFLD) despite its major impacts on public health. There are, however, technical challenges to developing therapeutics for treating NAFLD (or MAFLD) as described in detail herein. Such technical challenges include, but are not limited to, the complexity and intrinsic patient heterogeneity of the disease including genomic profile and phenotypic expression of the disease, the long length of disease progression (e.g., decades) in many patients, the complexity of the liver's physiology (including complex intra- and intercellular interactions), the complexity of the disease processes, the large number of DEGs and/or differentially enriched pathways relevant to the disease and their mechanistic roles with respect to the disease processes. The QSP methods described herein provide solutions to these technical challenges and as a result can be used to develop therapeutics for treating NAFLD (or MAFLD) when tested in microphysiology systems recapitulating the disease. Importantly, the DEGs, comprising the computationally derived signatures used to query the connectivity database to identify drugs, not only map to specific pathways but represent landmarks of disease processes independently implicated in NAFLD progression. The highest priority drugs that appear with the greatest frequency among the 12 signature-based queries (FIG. 10 (Table 1)) are predicted to robustly address the challenge imposed by the complex pathogenesis of NAFLD. This set of high priority drug predictions is enriched with drugs (e.g., isradipine, geldanamycin, vorinostat) having specific targets and the potential to engender normalizing pleiotropic effects on dysregulated physiology independently implicated in NAFLD. Thus, the integrated QSP methods described herein 1) identify the key characteristics of drugs predicted to address the complex pathophysiology of NAFLD and 2) provide the means to experimentally validate the relationship between drug efficacy, its mode-of-action, and disease mechanism.

The methods include analyzing RNA sequencing (RNA-seq) data for a plurality of patients having a disease, e.g., NAFLD (or MAFLD). The analysis identifies a plurality of differentially expressed genes (DEGs) and a plurality of differentially enriched biological pathways. This step is described in detail below, for example, in Example 1. The RNA-sequence data is derived from biopsies of livers of patients undergoing bariatric surgery, but can also be accessed from other patient cohorts. Each tissue sample is labeled according to the predominant liver histology of a patient (e.g., normal, steatosis, lobular inflammation, fibrosis) from which the tissue sample is obtained. It should be understood that biopsies are obtained and RNA-seq data is derived according to techniques known in the art. For example, gene expression levels can be obtained by sampling liver tissue, extracting RNA from the sample, sequencing the RNA, and quantifying the gene expression levels as compared to a control. This disclosure contemplates that data other than RNA-seq data can be analyzed. For example, other data may include, but is not limited to, metabolomic data, phosphoproteomic data, or other data. It should be understood that such other data can be analyzed in addition to RNA-seq data, or in some implementations without RNA-seq data.

In some implementations, the step of analyzing the RNA-seq data includes mapping a plurality of gene expression values to a plurality of biological pathway expression profiles; and associating the biological pathway expression profiles with the disease states of the disease (e.g., NAFLD). Disease states of NAFLD include, but are not limited to, entirely normal and steatosis (N&S), predominantly lobular inflammation (PLI), and predominantly fibrosis (PF). Optionally, the step of mapping the gene expression values to the biological pathway expression profiles includes using a gene set variation analysis (GSVA) algorithm. This step is described in detail below, for example, in Example 1. It should be understood that GSVA algorithm is provided only as an example. This disclosure contemplates using other algorithms for mapping the gene expression values to the biological pathway expression profiles. Alternatively or additionally, the step of associating the biological pathway expression profiles with the disease states of the disease (e.g., NAFLD) optionally includes using an unsupervised learning algorithm such as a clustering algorithm. This step is described in detail below, for example, in Examples 1 and 3. It should be understood that clustering is provided only as an example algorithm. This disclosure contemplates using other algorithms for mapping the gene expression values to the biological pathway expression profiles.

The methods include deriving a plurality of gene expression signatures associated with each of a plurality of disease states (e.g., entirely normal and steatosis, predominantly lobular inflammation, and predominantly fibrosis) of the disease (e.g., MAFLD) using the DEGs and the differentially enriched biological pathways. This step is described in detail below, for example, in Example 1. As described in the examples, each disease state is associated with a plurality of gene expression signatures. For example, the disease state entirely normal and steatosis (N&S) is associated with a plurality of gene expression signatures. The disease state predominantly lobular inflammation (PLI) is associated with a plurality of gene expression signatures. The disease state predominantly fibrosis (PF) is associated with a plurality of gene expression signatures. It should be understood that the gene expression signatures for each disease state are unique to the disease state. Additionally, entirely N&S, PLI, and PF are provided only as example disease states of MAFLD. It should be understood that more or less disease states of MAFLD than those provided as examples may be considered.

The methods also include identifying a plurality of drugs predicted to reverse a particular gene expression signature associated with a particular disease state (e.g., entirely normal and steatosis, predominantly lobular inflammation, and predominantly fibrosis) of the disease (e.g., NAFLD) and/or a physiological characteristic associated with a particular disease state (e.g., entirely normal and steatosis, predominantly lobular inflammation, and predominantly fibrosis) of the disease (e.g., NAFLD). For example, such physiological characteristics can include, but are not limited to, phenotypic measurements of steatosis, lobular inflammation, fibrosis, or other metric measured in the MPS disease model. This step is described in detail below, for example, in Examples 1, 4 and 5. In this step, drugs (or combinations of drugs) predicted to normalize one of the disease states are identified. As used herein, a drug is a chemical substance used in diagnosis, treatment, or prevention of disease. This disclosure contemplates that the drug is an existing drug that may be repurposed for diagnosis, treatment, or prevention of NAFLD. Alternatively, this disclosure contemplates that the drug is a novel drug developed for diagnosis, treatment, or prevention of NAFLD. Additionally, in some implementations, the identified drug is a single drug. In other implementations, the identified drug is a combination of drugs. In some implementations, the drug or combination of drugs includes a modulator directly acting on one or more targets with or without downstream pleiotropic effects to correct a particular disease state of the disease as described herein.

In some implementations, the step of identifying the drugs predicted to reverse the particular gene expression signature and/or physiological characteristic associated with the particular disease state of the disease includes using a connectivity map (CMap). This step is described in detail below, for example, in Examples 1, 4, and 5. It should be understood that using a connectivity map is provided only as an example algorithm. This disclosure contemplates using other algorithms for identifying the drugs.

The method further includes prioritizing for further experimental testing the drugs predicted to normalize the particular gene expression signature and/or physiological characteristic associated with the particular disease state of the disease. This step is described in detail in Example 1 below. The method described herein identifies drugs/drug combinations that can be either single state modulators or pleiotropic modulators targeting the disease states that can be defined by genomic reversion of the gene expression profiles, as well as key in vitro biomarkers of the pathophysiology. For example, a plurality of drugs (i.e., more than one drug) are predicted to normalize a disease state, and these drugs are then prioritized for further experimental testing to determine which of these drugs can be used to diagnose, treat, and/or prevent disease (e.g., NAFLD). In some implementations, a signature frequency ranking algorithm is used to prioritize drugs. For example, the drugs are prioritized based on their frequency of appearance across all gene expression signatures. It should be understood that a drug with higher frequency of appearance across all gene expression signatures may be useful for normalizing multiple disease states and therefore may be more promising (i.e., should be prioritized for further testing in the MPS NAFLD experimental model). This step is described in detail below, for example, in Example 1. In other implementations, a network mapping algorithm is used to prioritize drugs. This step is described in detail below, for example, in Examples 1 and 6. It should be understood that signature frequency ranking and network mapping are provided only as example algorithms. This disclosure contemplates using other algorithms for prioritizing drugs for further experimental testing.

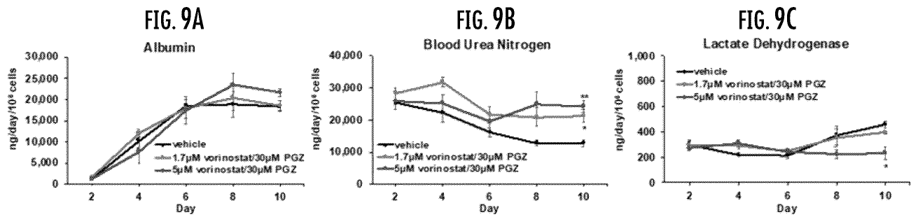

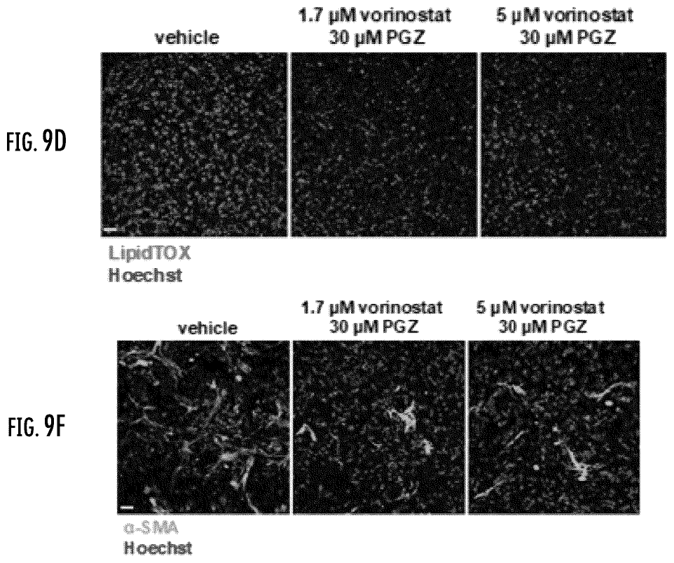

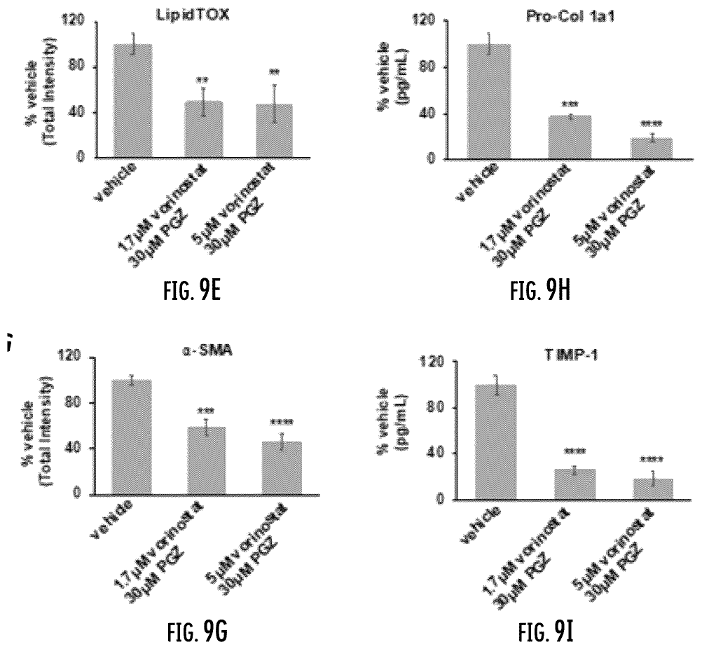

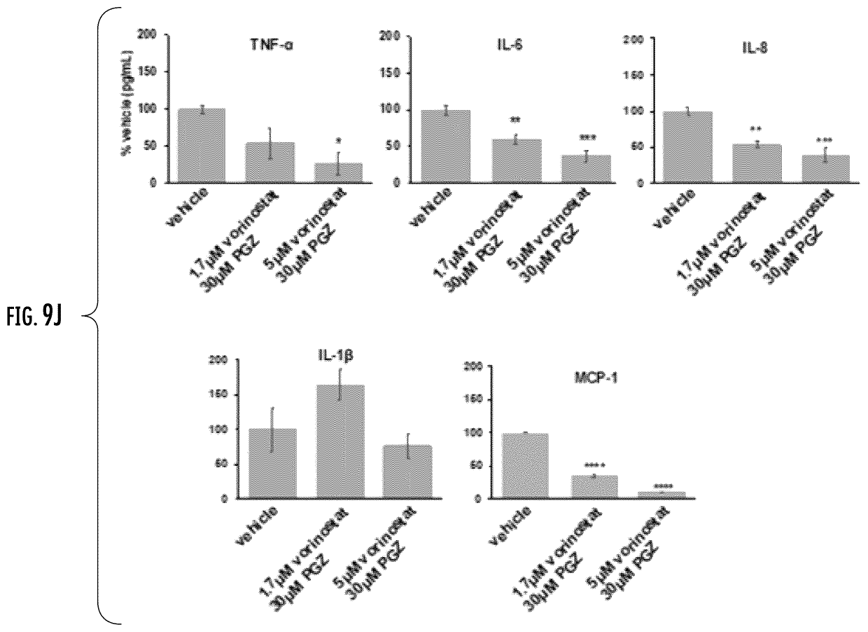

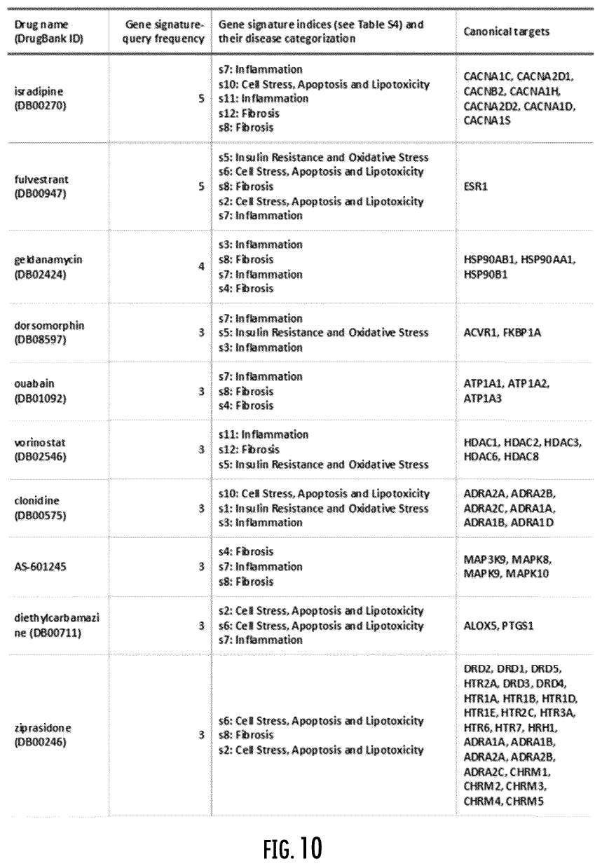

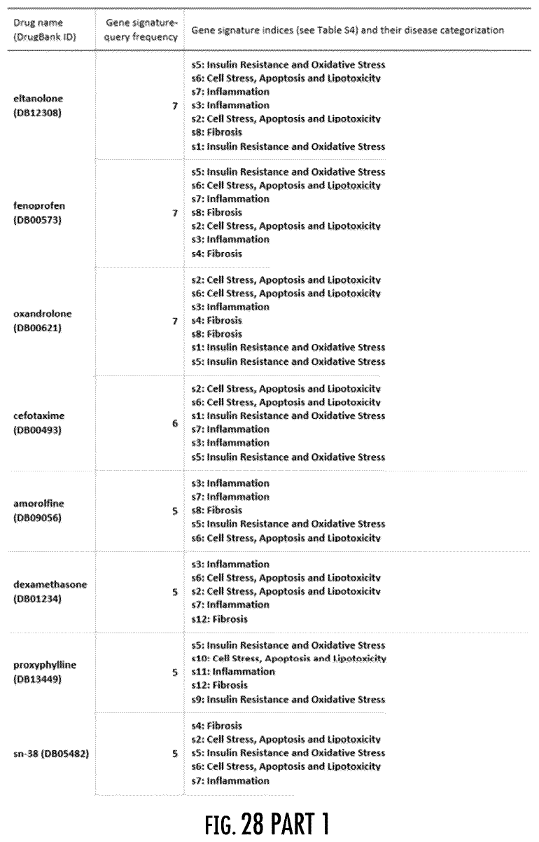

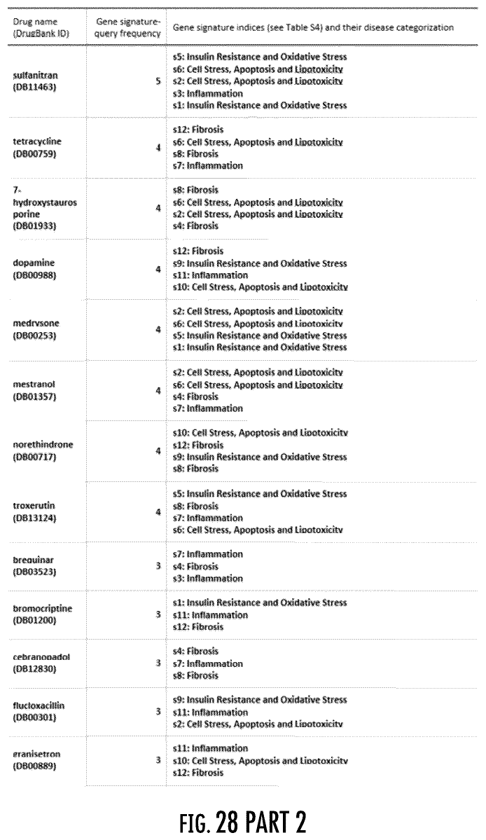

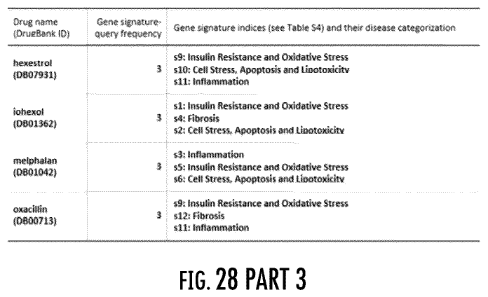

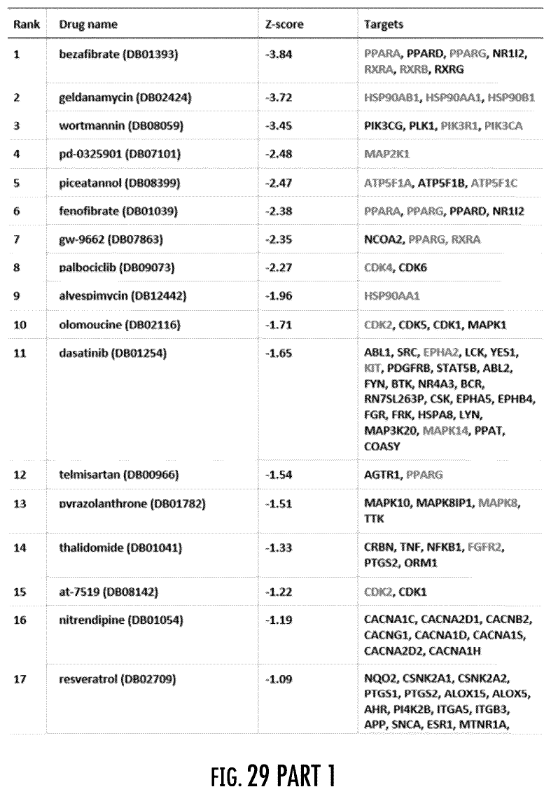

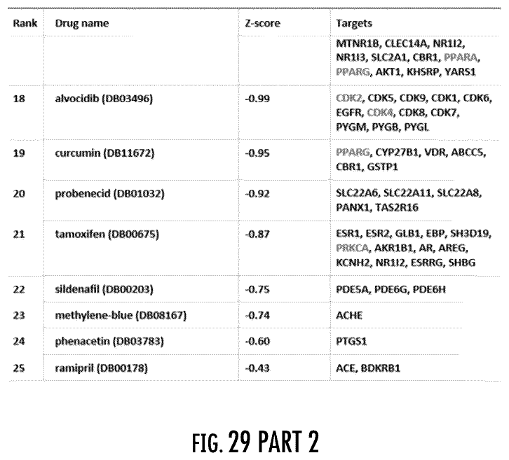

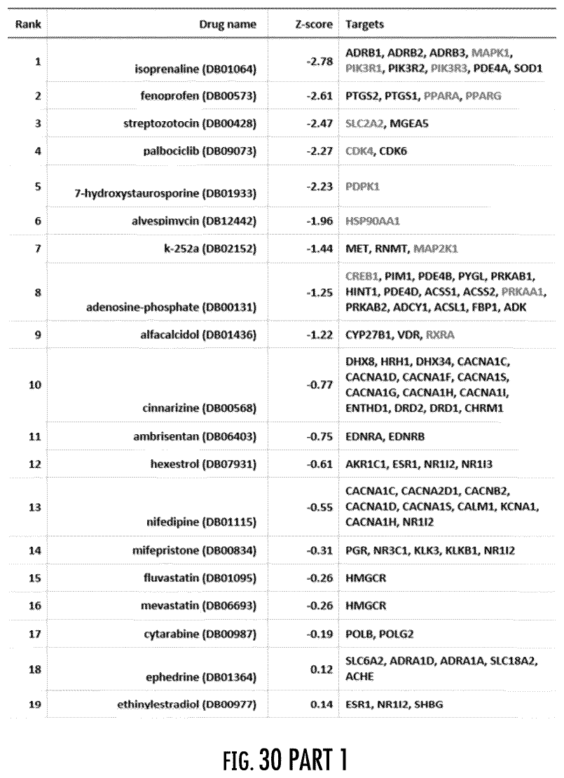

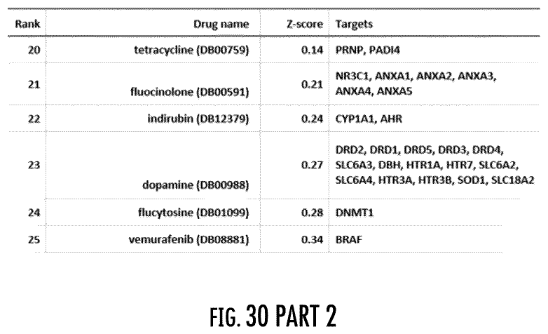

In contrast to specifically targeting individual nuclear receptors such as FXR, PPARs, and thyroid hormone receptors that are known to play an integral role in NAFLD pathophysiology with specific modulators that have dominated the current clinical trial landscape, the methods described herein select for structurally diverse endogenous ligands/xenobiotic entities (i.e., drugs) that can perturb the nuclear receptor network in an unbiased comprehensive manner to more completely reverse the disease state. Thus a single entity identified by these methods can in an unanticipated manner simultaneously act as an agonist for some members of the nuclear receptor network and as an antagonist for others to efficiently reverse the disease state. In addition to the aforementioned nuclear receptors, we have inferred (supported by the literature)) that the nuclear receptor PXR is a key target of these poly-pharmacologically acting drugs (e.g., eltanolone, SN-38, tetracycline from FIG. 28). Our identification of the HDAC inhibitor, vorinostat, and the HSP90 inhibitor, gledanamycin, are mechanistically consistent with this overall hypothesis as these drugs are canonical regulators of nuclear receptor function. Experimentally, the combination of vorinostat and the PPAR agonist, pioglitazone (FIG. 9), are more effective than each drug alone in reversing the disease state provides additional proof-of-concept for the methods described herein. The finding that this unbiased and comprehensive approach identifies approved drugs for a novel therapeutic indication suggests safety in addition to efficacy.

Optionally, the further experimental testing includes testing, using a human or animal microphysiological systems (MPS) platform, a drug or combination of drugs selected from the drugs predicted to normalize the particular gene expression signature associated with the particular disease state of the disease. This step is described in detail below, for example, in Examples 1, 2, and 7. The further experimental testing may include RNA sequencing and/or obtaining panels to measure physically relevant metrics. Optionally, the further experimental testing results in identification of one or more biomarkers for a disease state of the disease.

The method optionally further includes demonstrating, using a human or animal MPS platform, that the drugs predicted to normalize the particular gene expression signature and/or physiological characteristic associated with the particular disease state of the disease reverses or halts the progression of the disease (e.g., NAFLD). In other words, the MPS platform can be used to measure a panel of physiologically relevant metrics to demonstrate the inhibition or reversal of disease state in the models.

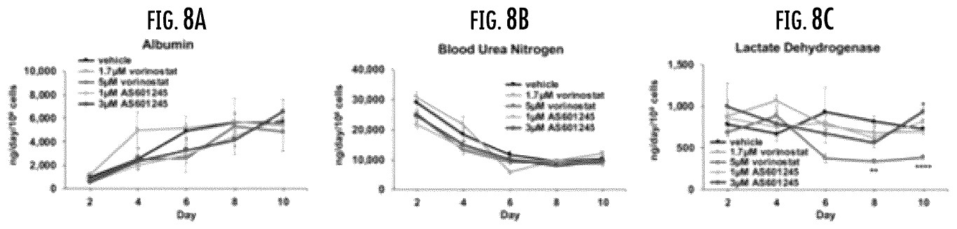

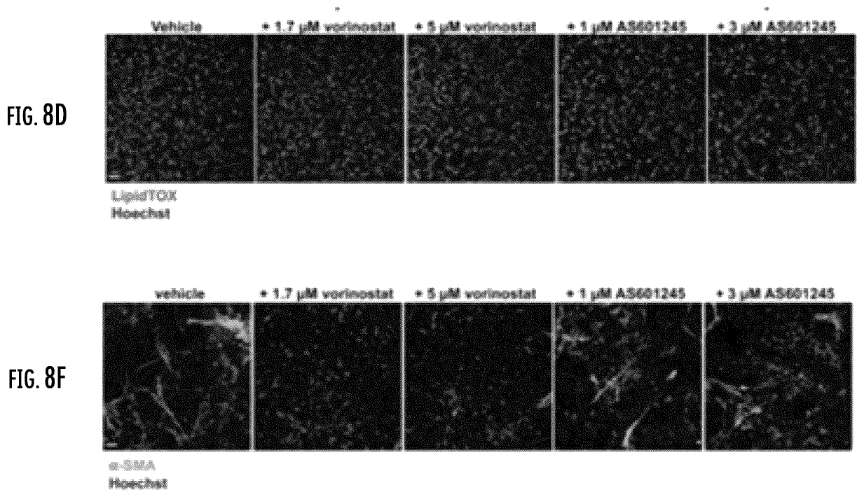

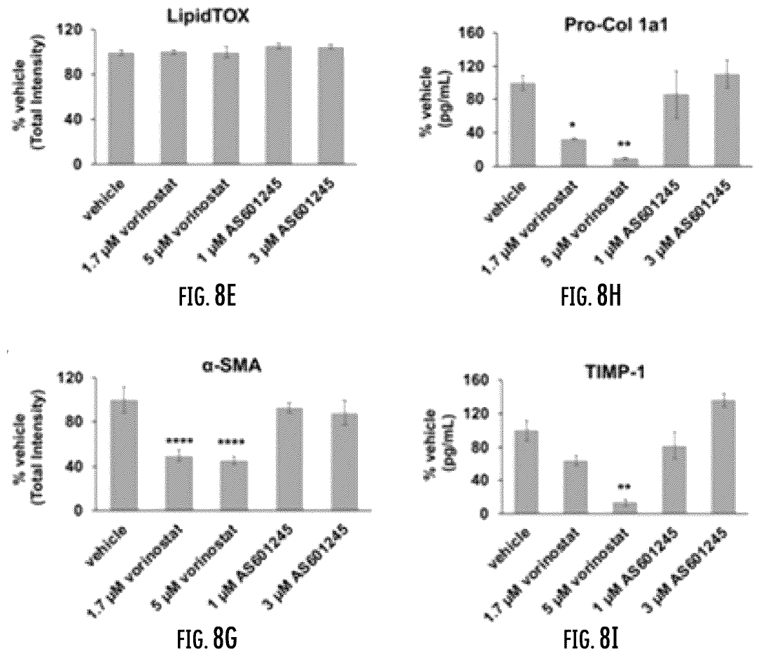

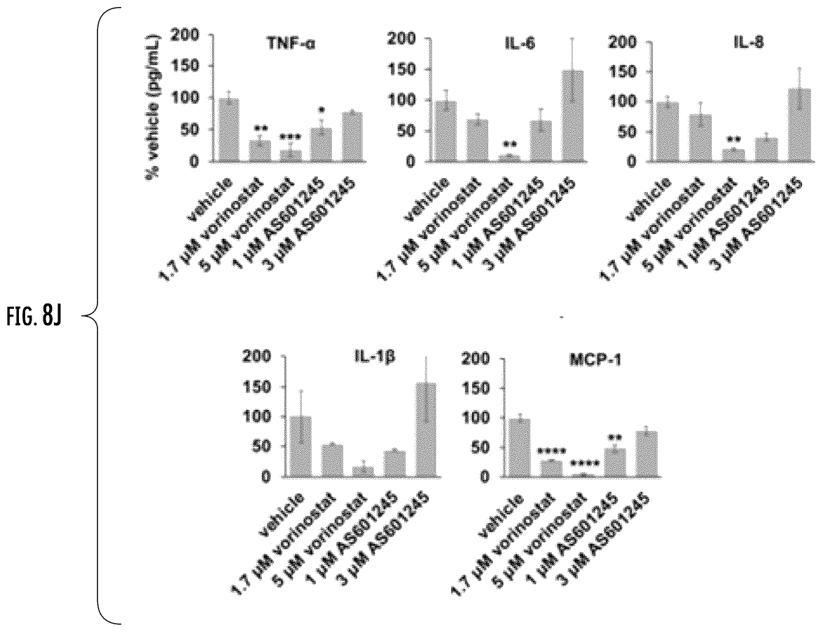

The method optionally further includes administering the drugs identified as described above to a subject in need thereof in an effective amount to decrease or inhibit the disease. As described herein, the subject may have NAFLD, and such drugs or combination of drugs are administered to treat NAFLD. For example, in some implementations, the subject having NAFLD is administered vorinostat (see e.g., FIG. 8). Alternatively, in some implementations, the subject having is administered a combination of pioglitazone and vorinostat (see e.g., FIG. 9).

In some implementations, the drugs predicted to normalize the particular gene expression signature and/or physiological characteristic associated with the particular disease state of the disease include a drug defined by Formula I, e.g. vorinostat:

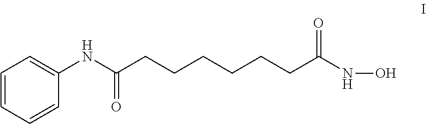 [SMILES: O=C(CCCCCCC(=O)Nc1ccccc1)NO]

or a pharmaceutically acceptable salt thereof.

In some implementations, the drugs predicted to normalize the particular gene expression signature and/or physiological characteristic associated with the particular disease state of the disease include a drug defined by Formula II, e.g. troglitazone:

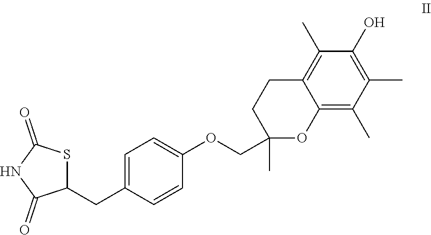 [SMILES: Cc1c(C)c2c(c(C)c1O)CCC(C)(COc1ccc(CC3SC(=O)NC3=O)cc1)O2]

or a pharmaceutically acceptable salt thereof

In some implementations, a subject diagnosed with NAFLD is treated with a drug defined by Formula I (e.g., vorinostat). In other implementations, the subject diagnosed with NAFLD is treated with a combination of drugs defined by Formula I (e.g., vorinostat) and Formula II (e.g., troglitazone).

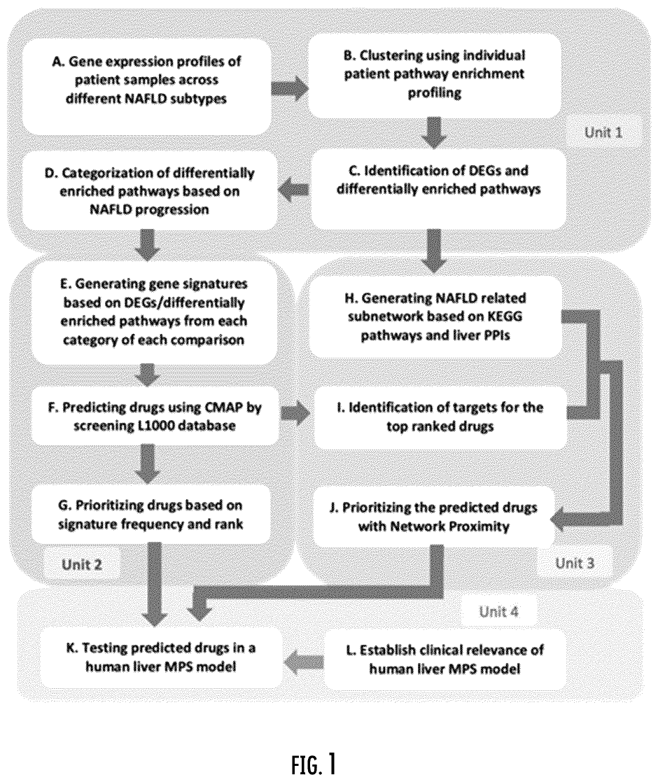

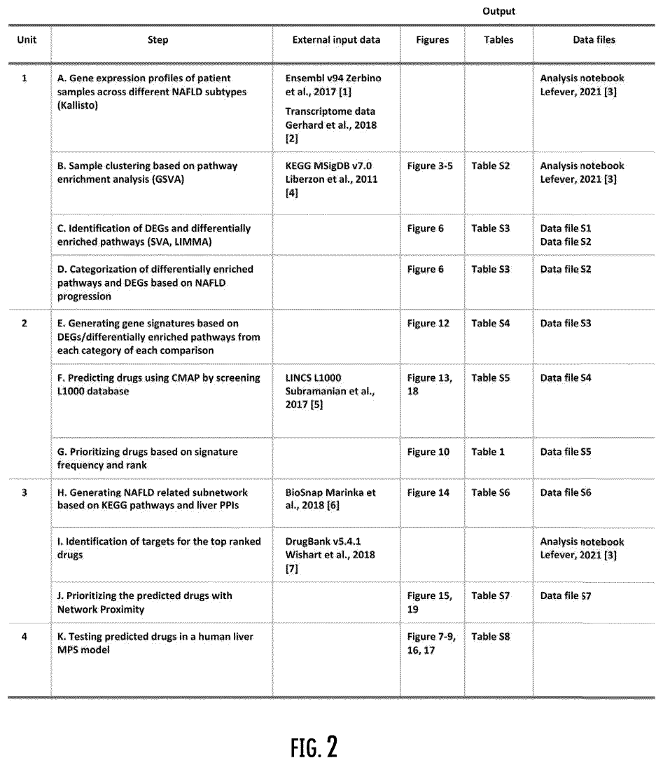

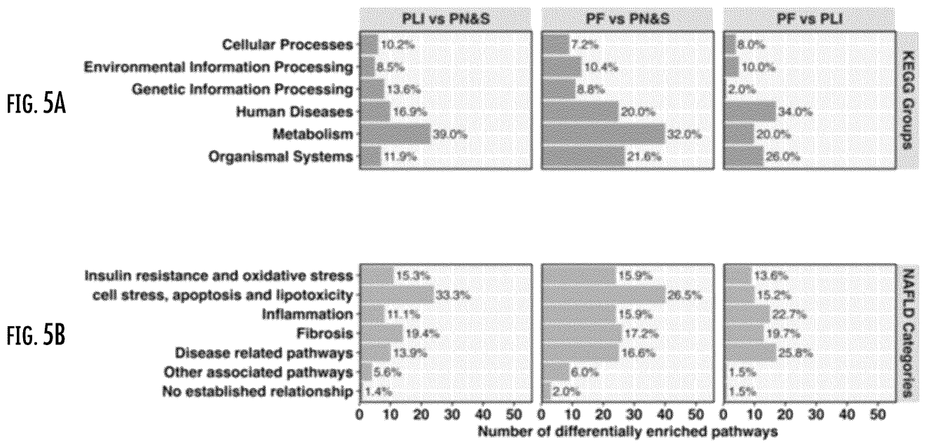

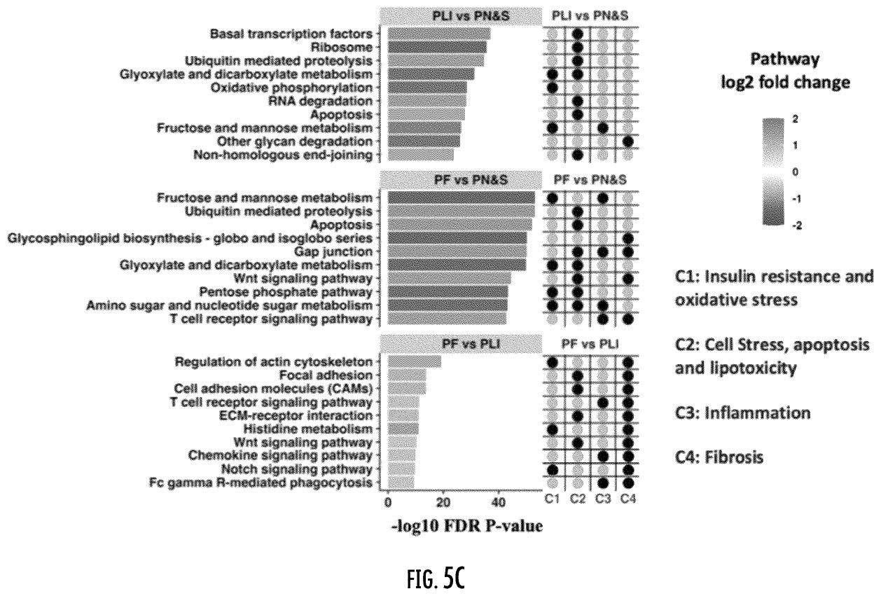

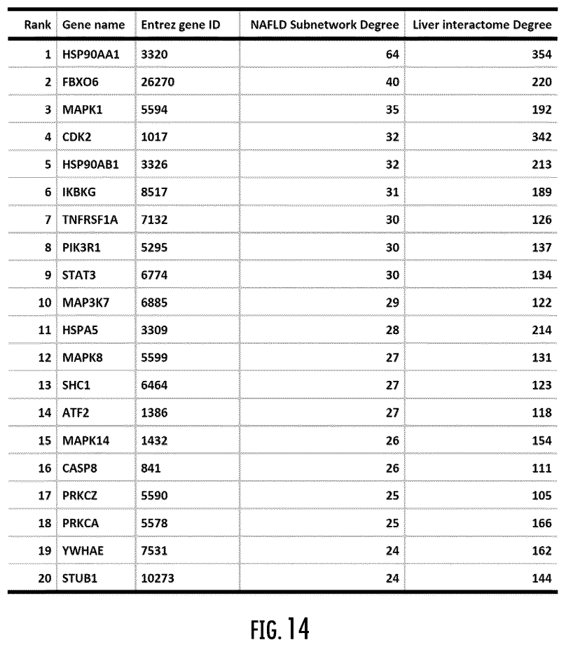

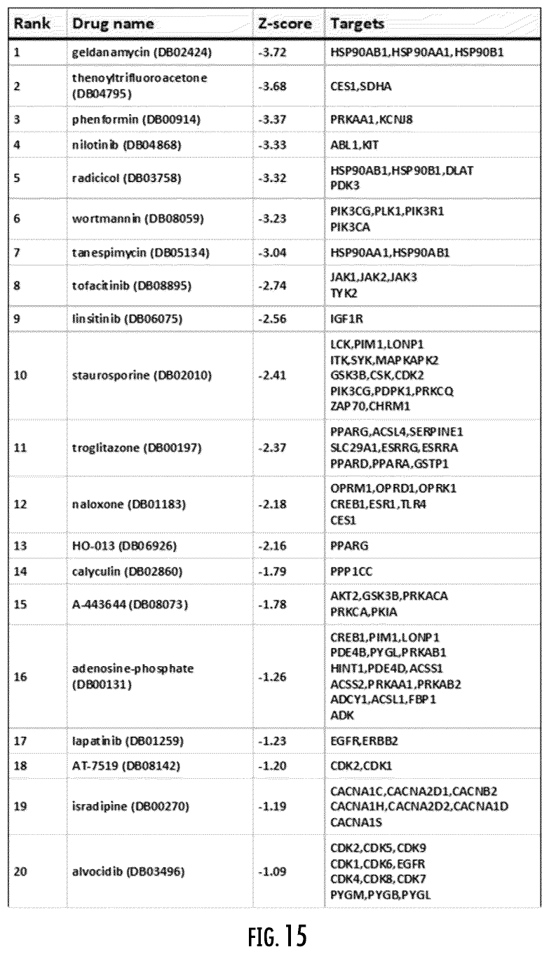

FIG. 1 is a workflow associating NAFLD subtypes with gene expression signatures to computationally predict and prioritize drugs for testing in a patient-derived microphysiological model of disease progression. Four integrated units are shown, each comprised of a set of steps detailed in Example 1 below. Unit 1 identifies and clusters individual patient hepatic gene expression and enriched pathway profiles associated with clinical subtypes and categorizes the differentially enriched pathways among these clusters within our current framework of NAFLD pathophysiology (FIGS. 3-6; Table S3, and Data files S1 & 2). Unit 2 generates disease progression-based gene expression signatures (Data file S3) and, through connectivity mapping (CMap), identifies drugs that can normalize these signatures (Table 1; Table S4, and Data file S4 & 5). The highly integrative Unit 3 maps known protein targets of the predicted drugs from Unit 2 to a NAFLD subnetwork encompassing protein targets from the gene expression analysis within Unit 1 (FIG. 14-15; Data files S6 & 7). A network proximity score is then calculated that increases the specificity of the CMap analysis to improve the prioritization of the predicted drugs for experimental testing (Table S6; Data file S7). In Unit 4, the effects of the prioritized drugs on a diverse set NAFLD-associated biomarkers in a human microphysiological systems (MPS), recapitulating aspects of NAFLD progression, are determined (FIG. 5).

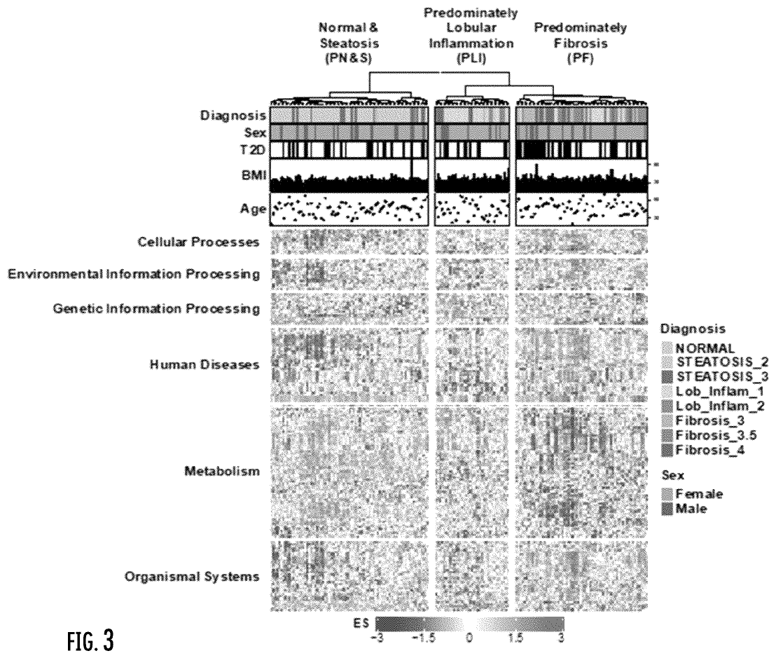

FIG. 2 is Table S1 which provides a cross reference to the Figures, Tables and Data Files that are output from each step in the procedure illustrated in FIG. 1.

FIG. 3 illustrates that individual patient liver transcriptome analysis yields distinct clusters based on their Kyoto Encyclopedia of Genes and Genomes (KEGG) pathway enrichment profiles. The heatmap shows hierarchical clustering of the liver KEGG pathway enrichment profiles (columns) from individual patients, determined by RNA sequencing and gene set variation analysis (GSVA) using MSigDB v7.0 C2 KEGG pathways (see Example 1 below). Pathways (rows) are grouped according to the top-level KEGG hierarchical classifications (labeled along the left ordinate) to which they belong. The color represents the enrichment score (ES; see color-coded bar under the heatmap), which reflects the degree to which a pathway is over or under-represented within that individual patient sample (see (Hanzelmann, Castelo et al. 2013)). The plots above the heatmap show the patient metadata: the top 2 bars indicate the color-coded diagnosis and patient sex, the third indicates if the patient has been diagnosed with type 2 diabetes (T2D) (black marks), and the additional two plots show the body mass index (BMI) and age of the patient. The three column clusters are named according to their predominant clinical classification: the first is almost entirely normal & simple steatosis (N&S), the second is predominately lobular inflammation (PLI), and the third is predominately Fibrosis (PF). Details of clinical subtype distribution for each cluster are shown in Table S1.

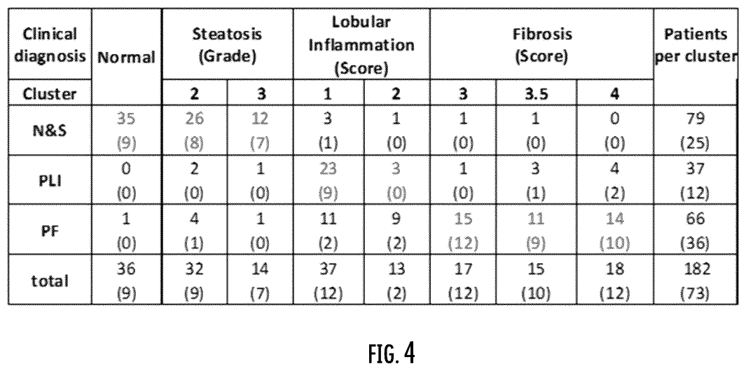

FIG. 4 is Table S2. Distribution of NAFLD patient subtypes within the three clusters defined in FIG. 3. The number of patients diagnosed with T2D are indicated in parentheses. The clustering of the cohort samples are significantly associated (Pearson's Chi-squared Test) with NAFLD subtype (p<2.2e-16) and T2D status (p=0.01). The red values denote the predominate subtype within each cluster.

FIGS. 5A-5C illustrate the distribution of differentially enriched pathways and their respective KEGG groups and NAFLD categories among the pairwise cluster comparisons defined in FIG. 3. The number of differentially enriched pathways identified between the PLI vs N&S, PF vs N&S, and PF vs. PLI pairwise comparisons were 59, 125, and 50, respectively (adj, p-value<0.001). Their distribution (and percent contribution) with respect to KEGG Groups (FIG. 5A) and NAFLD categories (FIG. 5B) are detailed in Table S3 and Data file S2. The top ten differentially enriched pathways for each comparison (ranked by the FDR adjusted p values through the linear modelling equivalent of a two sample, moderated t-test) are shown along with their association (bolded circles) with NAFLD categories C1-4 (as indicated and defined in the Examples below) (FIG. 5C). The colors of the bars represent the directionality and relative enrichment of each pathway for each of the pairwise comparisons.

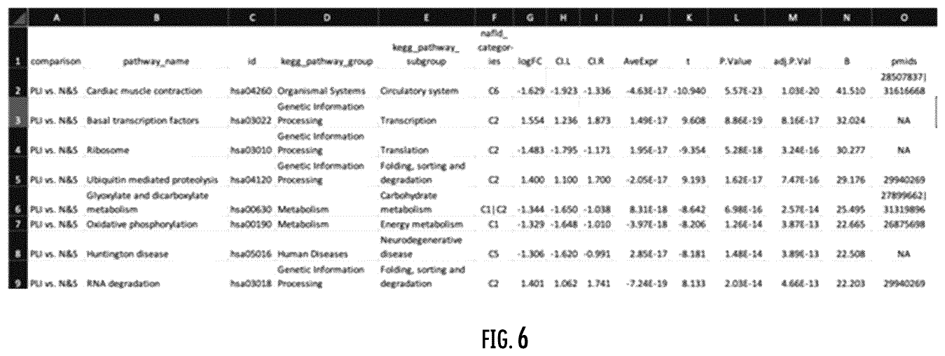

FIG. 6 is an except of Table S3. The differentially enriched pathways across 7 NAFLD categories for three pairwise cluster comparisons (supporting FIG. 5). Data file S2 was used to create Table S3. Table S3 consists of 3 sheets: PLI vs. N&S, PF vs. N&S, and PF vs. PLI comparisons. The columns of the tables are as follows:
KEGG Pathway name and ID
KEGG pathway group
KEGG pathway subgroup
NAFLD categorization of KEGG pathway (see Methods)
   C1: Insulin resistance and oxidative stress
   C2: cell stress, apoptosis and lipotoxicity
   C3: Inflammation
   C4: Fibrosis
   C5: Disease related pathways
   C6: Other associated pathways
   C7: No established relationship
log 2 Fold change: estimate of the log 2-fold-change of the comparison
FDR corrected p-value: False discovery rate
PMIDs: The PMIDs for the references which support the NAFLD categorization

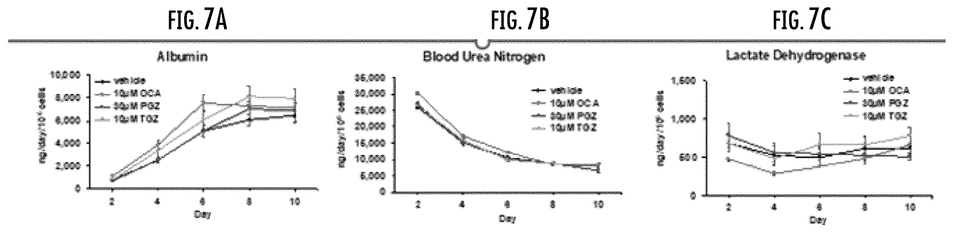

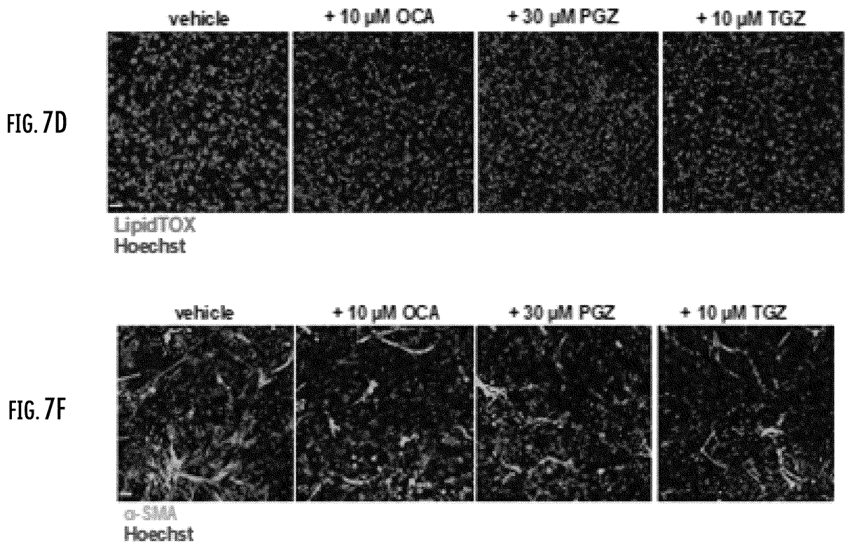

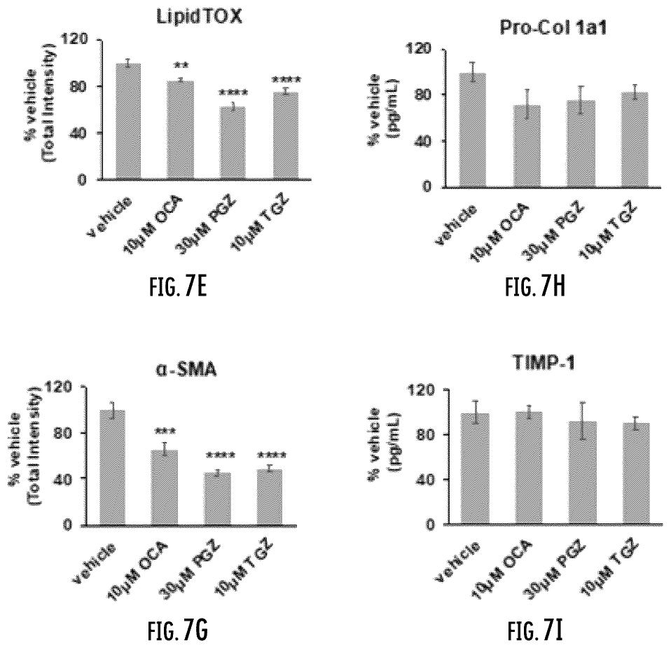

FIGS. 7A-71 illustrate how Obeticholic acid, Pioglitazone and Troglitazone reduce both lipid accumulation and stellate cell activation in LAMPS models treated with NAFLD disease media. LAMPS models were maintained for 10 days in NAFLD disease media containing either 10 μM obeticholic acid (OCA), 30 μM Pioglitazone (PGZ), 10 μM Troglitazone (TGZ), or DMSO vehicle control and a panel of metrics were examined to monitor disease-specific phenotypes. (FIG. 7A-C) Albumin, blood urea nitrogen, and lactate dehydrogenase curves throughout the 10-day time course show no significant differences between vehicle and drug treatment groups, suggesting no overt model cytotoxicity and loss of function. At day 10, there is a significant decrease in hepatocellular steatosis (FIG. 7D; LipidTOX intensity) and stellate cell activation (FIG. 7E; α-SMA intensity) in the OCA, TGZ and PGZ treatment groups compared to vehicle control. Although there is a ˜20% decrease in secretion of the pro-fibrotic marker Pro-Col 1a1 (FIG. 7F) with treatment of OCA, TGZ or PGZ, this decrease is not significant. In addition, there is also no significant change in the secreted levels of TIMP-1 in any of the treatment groups compared to vehicle (FIG. 7G). n=3 chips were analyzed for each assay and statistical significance was assessed using a One-Way ANOVA with Dunnett's test to make comparisons between each drug treatment group and the vehicle control (** p<0.01; *** p<0.001; **** p<0.0001).

FIGS. 8A-8J illustrate that the HDAC inhibitor Vorinostat reduces stellate cell activation, the secretion of both pro-fibrotic markers and inflammatory cytokines in LAMPS models treated with NAFLD disease media. LAMPS models were maintained for 10 days in NAFLD disease media containing either Vorinostat (1.7 μM or 5 μM), AS601245 (1 μM or 3 μM), or DMSO vehicle control and a panel of metrics were examined to monitor disease-specific phenotypes. Albumin and blood urea nitrogen curves show no significant differences between vehicle and drug treatment groups (FIG. 8A & FIG. 8B), suggesting that these drug treatments do not result in loss of model functionality. However, there is a significant decrease in LDH secretion (FIG. 8C) at days 8 and 10 in the 5 μM Vorinostat treatment group, demonstrating that treatment with this drug alleviates cytotoxicity. This result is further supported by the overall significant decrease in the day 10 measurements of stellate cell activation (FIG. 8E; α-SMA intensity), production of the pro-fibrotic markers pro-collagen 1a1 and TIMP-1 (FIG. 8F & FIG. 8G) and inflammatory cytokine production (FIG. 8H) observed in the vorinostat treatment group. In addition, within the AS601245 treatment group, we observe a general dose-dependent increase in the secretion of each of the cytokines assayed, suggesting that treatment with the higher dose of this compound results in an uptick of inflammatory signaling in the model (FIG. 8H). This is supported by the significant increase in day 10 LDH compared to the vehicle control for 3 uM AS601245 (FIG. 8C). Neither Vorinostat or AS601245 treatment alleviates lipid accumulation at day 10 compared to vehicle control (FIG. 8D), indicating no effect on steatosis. n=3 chips were analyzed for each assay and statistical significance was assessed using a One-Way ANOVA with Dunnett's test to make comparisons between each drug treatment group and the vehicle control (** p<0.01; *** p<0.001; **** p<0.0001).

FIGS. 9A-9J illustrate that pioglitazone and vorinostat used in combination results in the reduction of steatosis and stellate cell activation as well as the secretion of pro-fibrotic markers and production of inflammatory cytokines in LAMPS models treated with NAFLD disease media. While albumin secretion profiles show no significant differences between vehicle and drug treatment groups, suggesting that these drug combinations do not result in loss of model functionality (FIG. 9A), a significant increase in urea nitrogen secretion is observed in both drug combination groups compared to control, suggesting increased model metabolic activity (FIG. 9B). In addition, like the LDH profile in FIG. 7, there is a significant decrease in LDH secretion (FIG. 9C) in the 5 μM vorinostat treatment group, suggesting a reduction in cytotoxicity. In contrast to the individual drug testing studies shown in FIGS. 7 and 8, we observe an overall decrease in both lipid accumulation (FIG. 9D & FIG. 9E) and stellate cell activation (FIG. 9F & FIG. 9G), as well as in the production of the pro-fibrotic markers pro-collagen 1a1 and TIMP-1 (FIG. 9H & FIG. 9I) and inflammatory cytokine production (J) when pioglitazone and vorinostat are used in combination. Panels FIG. 9D & FIG. 9F display representative 20× Day 10 LIPIDTOX (9D) and α-SMA images of LAMPS under each treatment condition; Scale bar; 50 μm. For each control and drug treatment group, n=3 chips were analyzed and plotted±SEM for each assay and statistical significance was assessed using a One-Way ANOVA with Dunnett's test to make comparisons between each drug treatment group and the vehicle control (** p<0.01; *** p<0.001; **** p<0.0001).

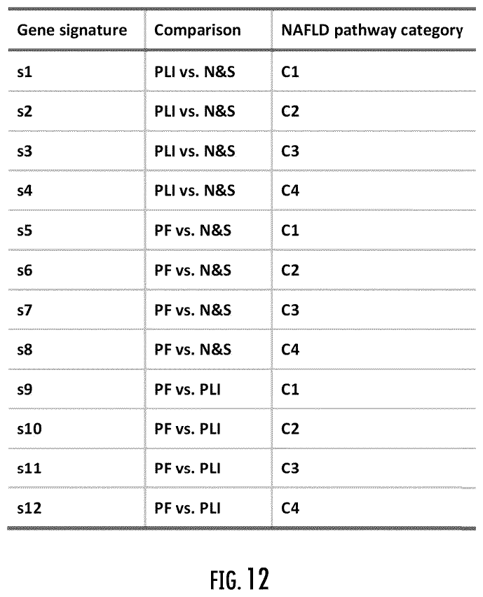

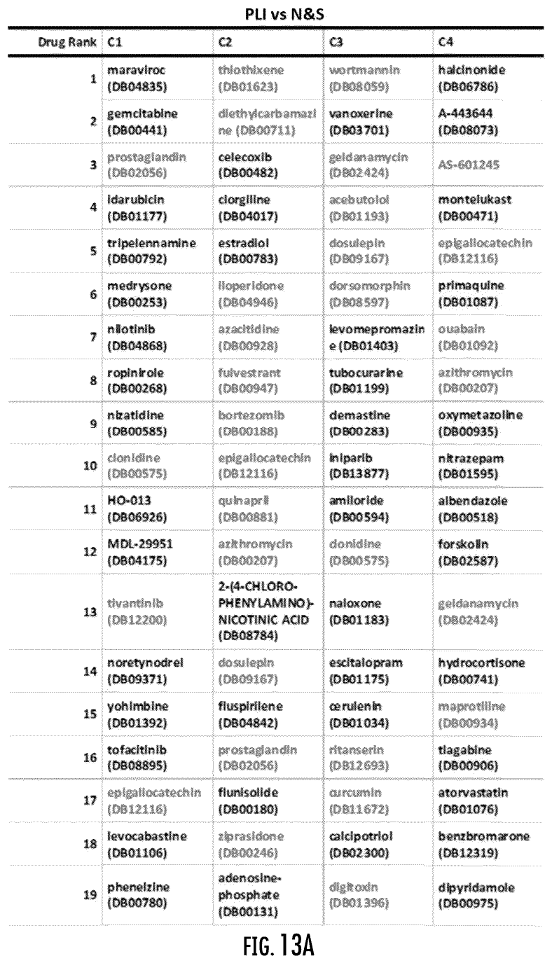

FIG. 10 is Table 1. Prioritization of CMap-predicted drugs based on occurrence across multiple MAFLD-associated gene signature queries. Drugs/small molecules perturbagens identified in more than 1 gene signature-based query were prioritized based on the number of occurrences (FDR p-value<0.05) across the 12 queries and termed: Gene signature-query frequency (FIGS. 12 & 13, Table S4 & S5; Data File S3 & S4). Each signature-based query is indexed S1-12 (see Table S4 and Data file S3) and ordered (from highest to lowest) according to the relative rank of the drug within each query that the drug was identified (i.e., occurrence). Each gene signature-based query is associated with a predominate feature (i.e., disease category) of MAFLD. The canonical targets derive from DrugBank (v5.1.4).

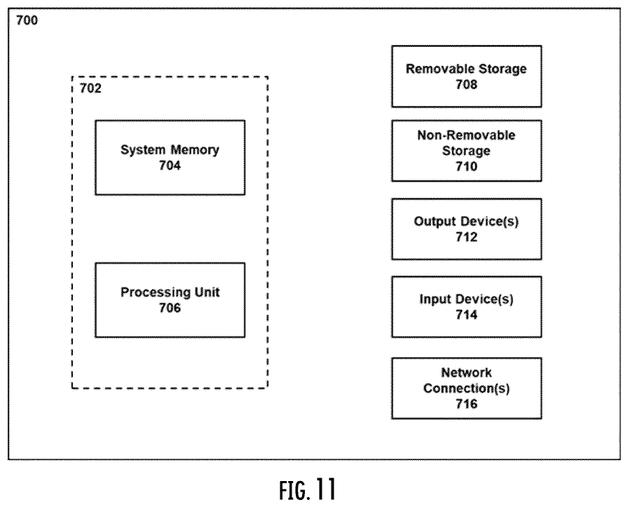

Referring to FIG. 11, is an example of a computing device 700 upon which the methods described herein may be implemented is illustrated. It should be understood that the example computing device 700 is only one example of a suitable computing environment upon which the methods described herein may be implemented. Optionally, the computing device 700 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 700 typically includes at least one processing unit 706 and system memory 704. Depending on the exact configuration and type of computing device, system memory 704 may be volatile (such as random access memory (RAM)), non-volatile (such as read-only memory (ROM), flash memory, etc.), or some combination of the two. This most basic configuration is illustrated in FIG. 11 by dashed line 702. The processing unit 706 may be a standard programmable processor that performs arithmetic and logic operations necessary for operation of the computing device 700. The computing device 700 may also include a bus or other communication mechanism for communicating information among various components of the computing device 700.

Computing device 700 may have additional features/functionality. For example, computing device 700 may include additional storage such as removable storage 708 and non-removable storage 710 including, but not limited to, magnetic or optical disks or tapes. Computing device 700 may also contain network connection(s) 716 that allow the device to communicate with other devices. Computing device 700 may also have input device(s) 714 such as a keyboard, mouse, touch screen, etc. Output device(s) 712 such as a display, speakers, printer, etc. may also be included. The additional devices may be connected to the bus in order to facilitate communication of data among the components of the computing device 700. All these devices are well known in the art and need not be discussed at length here.

The processing unit 706 may be configured to execute program code encoded in tangible, computer-readable media. Tangible, computer-readable media refers to any media that is capable of providing data that causes the computing device 700 (i.e., a machine) to operate in a particular fashion. Various computer-readable media may be utilized to provide instructions to the processing unit 706 for execution. Example tangible, computer-readable media may include, but is not limited to, volatile media, non-volatile media, removable media and non-removable media implemented in any method or technology for storage of information such as computer readable instructions, data structures, program modules or other data. System memory 704, removable storage 708, and non-removable storage 710 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 706 may execute program code stored in the system memory 704. For example, the bus may carry data to the system memory 704, from which the processing unit 706 receives and executes instructions. The data received by the system memory 704 may optionally be stored on the removable storage 708 or the non-removable storage 710 before or after execution by the processing unit 706.

It should be appreciated that the logical operations described herein with respect to the various figures may be implemented (1) as a sequence of computer implemented acts or program modules (i.e., software) running on a computing device (e.g., the computing device described in FIG. 11), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

FIG. 12 is Table S4. Gene signature index. The 12 gene signatures (Data file S3) are composed of a unique combination of the 3 NAFLD subclass pairwise comparisons and 4 NAFLD pathway categories (see Example 1 below; FIG. 18 for details on the methodology). The NAFLD pathway categories are: insulin resistance and oxidative stress (C1), cell stress, apoptosis and lipotoxicity (C2), inflammation (C3), and fibrosis (C4) (see FIG. 5; Table S3; and Data file S2 for the distribution and details of these pathways in the pairwise comparisons).

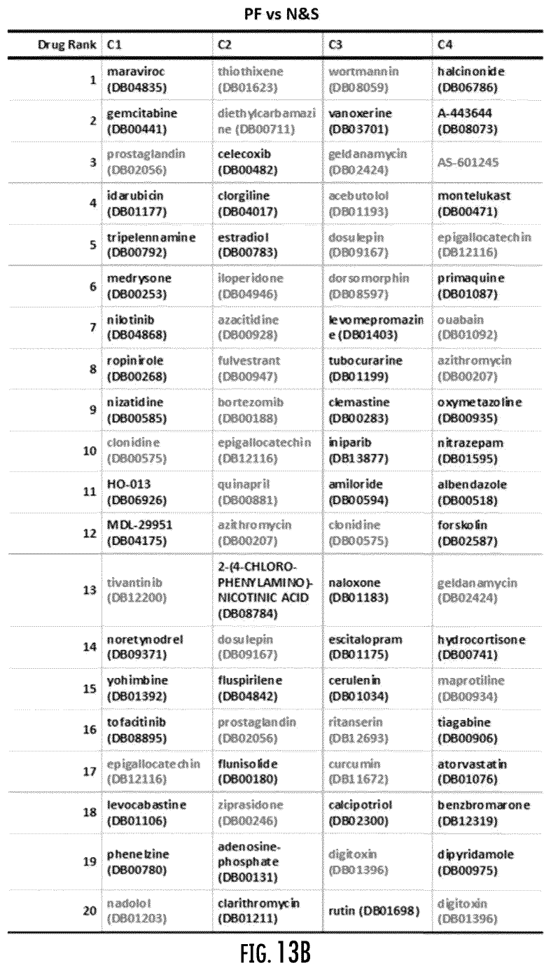

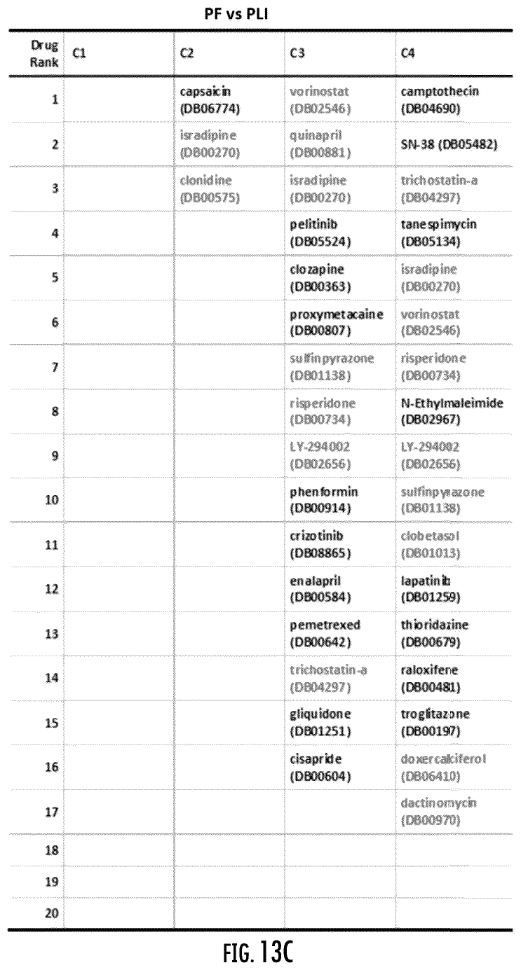

FIGS. 13A-13C is Table S5. The top 20 drug and small molecule perturbagen predictions from CMap analysis for each of the 12 queries. The predictions were first filtered to remove those with FDR p-value>0.05. Missing entries indicate that the query had <20 predictions which met this statistical criterion. Perturbagens (DrugBank v5.1.4 identification) colored in red are those that appear in the top 20 predictions for >1 signature. Results were generated using Data file S5 and support (FIG. 10) Table 1.

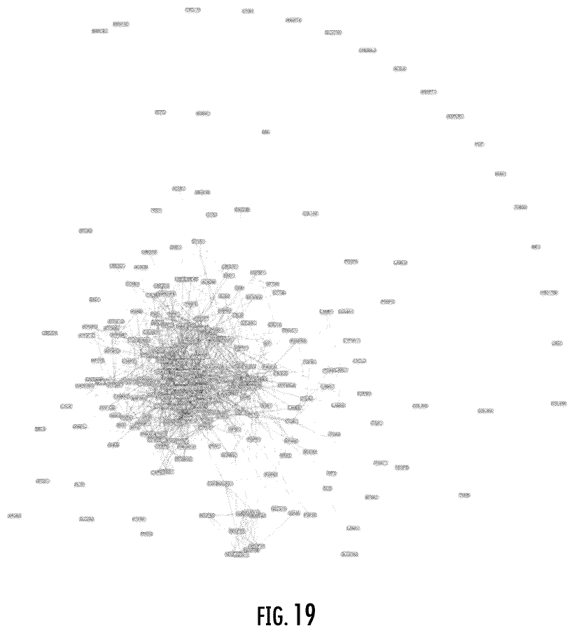

FIG. 14 is Table S6. The top 20 hubs ranked by degree in the NAFLD subnetwork. The hubs are indicated by gene name and the degree is defined by the number of interactions with proteins encoded by other NAFLD DEGs. For comparison, the degree of the hub is also indicated in the context of the background human liver protein-protein interactome. Table S6 was generated using Data file S6 and provides additional detail to FIG. 19.

FIG. 15 is Table S7. Prioritization of CMap-predicted drugs and small-molecule perturbagens based on NAFLD subnetwork proximity. Table S7 is derived from Data file S7 and supports FIG. 7. The common name of the drug/small molecule with the DrugBank ID in parenthesis is shown. The Z-scores were calculated as described in the Methods and Guney et al. (Guney, Menche et al. 2016). The drug targets are those listed in DrugBank (v5.1.4).

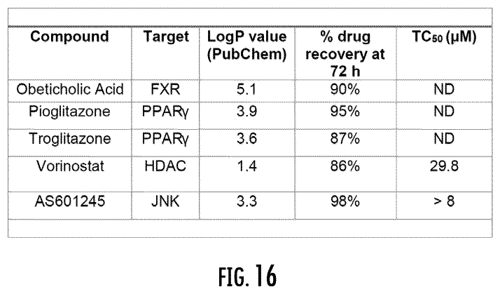

FIG. 16 is Table S8. Drug binding and cytotoxicity profiles for compounds used in LAMPS studies. To assess the drug binding capability of the polydimethylsiloxane (PDMS)-containing LAMPS device for compounds used in these studies, we used perfusion flow tests and mass spectrometry analysis of efflux collected from LAMPS devices at 72 h to determine the overall effective concentration of each compound compared to the starting concentration of drug as previously described (Vernetti, Senutovitch et al. 2016, Miedel, Gavlock et al. 2019). The TC₅₀ (Toxic Concentration inducing 50% hepatocyte death) was determined in a 5-day hepatocyte cytotoxicity assay. ND—not determined. The TC₅₀ assay was not conducted on Obetacholic acid, Pioglitazone or Troglitazone. The concentration of these compounds was based on previous experimentation in the LAMPS models.

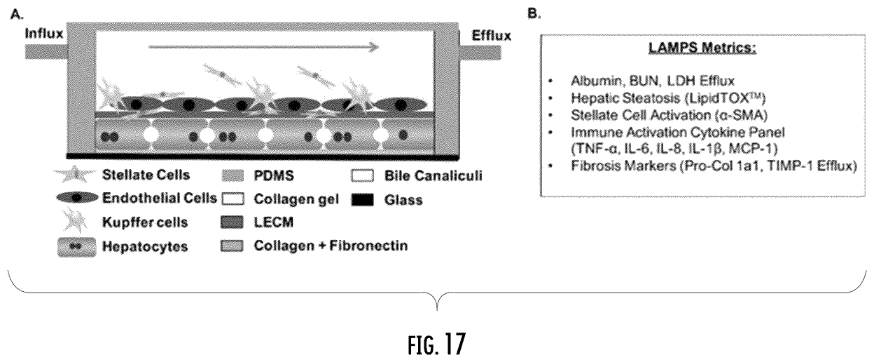

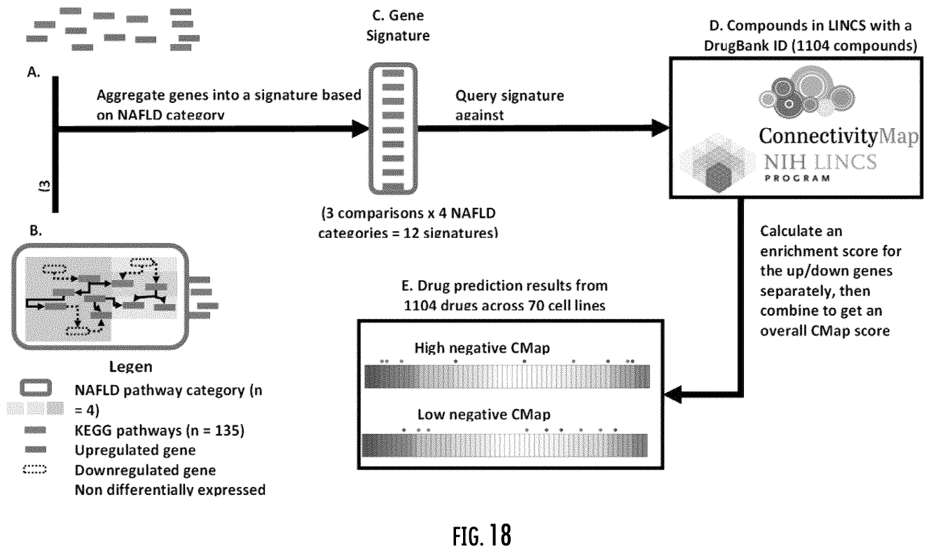

FIG. 17 illustrates the Biomimetic Human Liver Acinus MicroPhysiology System (LAMPS) used for proof-of-concept experimental testing of CMap-predicted drugs.

FIG. 18 illustrates a description of CMap methodology. Gene signatures (C) were used to query the LINCS database (D, See Example 1 and text below for detail). The signature is a set of up- and down-differentially expressed genes between disease versus non-disease states. The overall objective is to identify drugs that normalize the gene expression pattern of the disease state to ameliorate the pathogenic phenotype.

These signatures are generated by first identifying differentially expressed genes (DEGs) and differentially enriched pathways between different states of disease progression. The differentially enriched pathways were categorized according to their known association with NAFLD progression (see FIG. 5). DEGs present only in differentially enriched pathways (18B, shown as light blue, pink, and green boxes) were used. This excluded differentially expressed genes that were not in a non-differentially enriched pathway and genes within the differentially enriched pathway which were not also differentially expressed. Gene signatures were generated by aggregating the up- and down-differentially expressed genes from different differentially enrich pathways belonging to a particular NAFLD pathway category (C1-4 as defined in FIG. 5). In the example shown, there are 3 pathways (18B, shown as light blue, pink, and green boxes which all belong to 1 NAFLD pathway category in the gray rounded box). There are 4 upregulated and 5 downregulated genes which were then combined to create a gene signature for that NAFLD pathway category (18C).

We then query the LINCS database of annotated perturbation signatures using the gene signature. This is done by calculating an enrichment score for the up and down regulated genes of the gene signature against the perturbation signatures (Subramanian, Narayan et al. 2017, Keenan, Jenkins et al. 2018). A composite bidirectional CMap score is then derived by averaging the difference of these 2 enrichment scores if the signs are different. In the case where the bidirectional criterion is not met, the CMap score is set to 0 thereby deprioritizing such drugs.

The output is a list of CMap scores for the query gene signature versus the perturbation signatures. These can be ranked according to this score. Since the objective is to reverse the query gene signature (i.e., negative connectivity), we focused on drugs that have the highest negative CMap score. Two examples are shown (18E), the dots represent genes from a query gene signature and the heatmap vector represents the gene expression from the perturbation signatures. In the first case the CMap score is high, since there are 3 upregulated genes from the query signature located towards the bottom of the perturbation signature. Conversely, the second case shows the query genes are neither focused towards the top or bottom of the perturbation signature.

FIG. 19 illustrates a NAFLD associated protein interactome. A subnetwork of the human liver protein interactome involving NAFLD associated protein-protein interactions. The indicated nodes represent those proteins encoded by the DEGs among the pairwise comparisons for the three clusters defined in FIG. 3. The degrees of these nodes are shown in Data file S6 and the 20 hubs with the highest degrees are shown in Table S6.

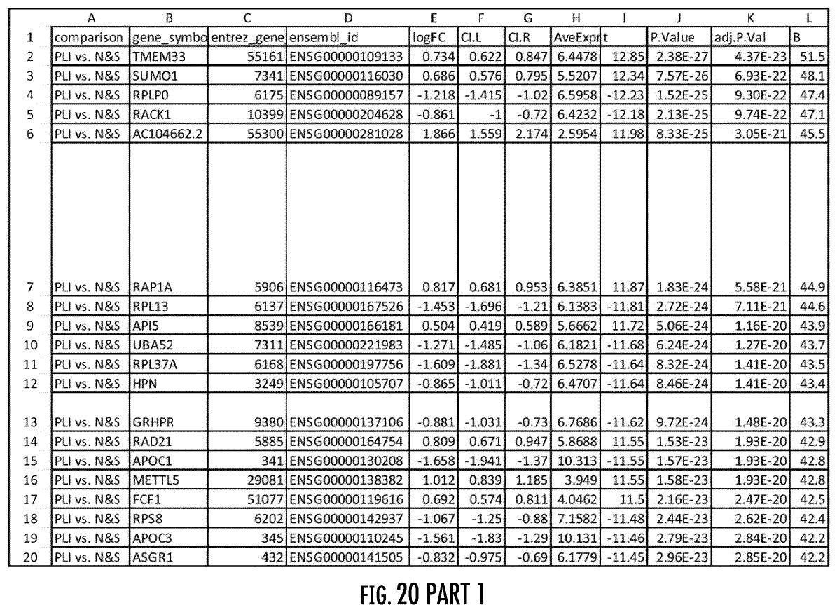

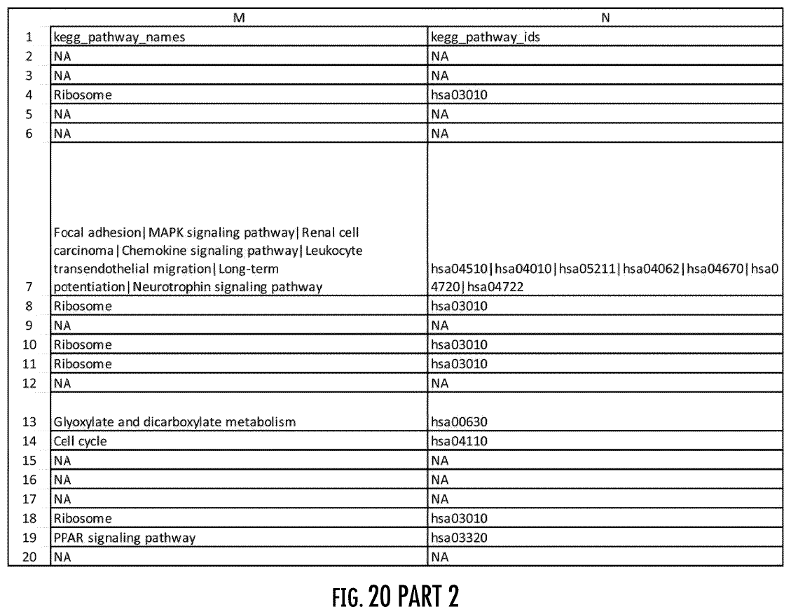

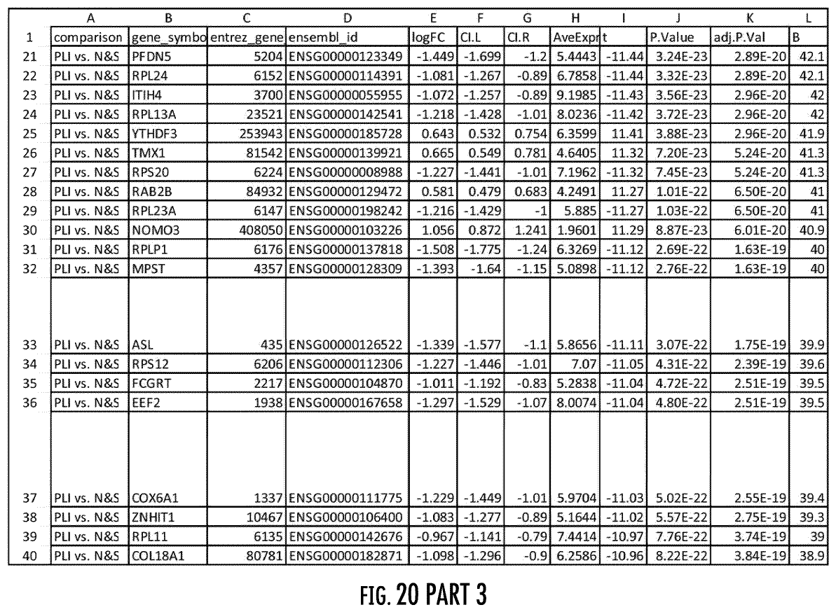

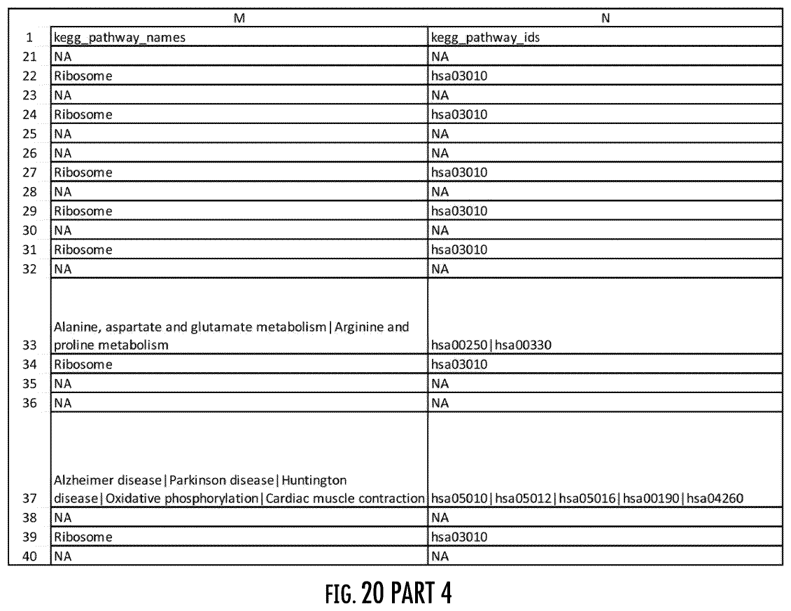

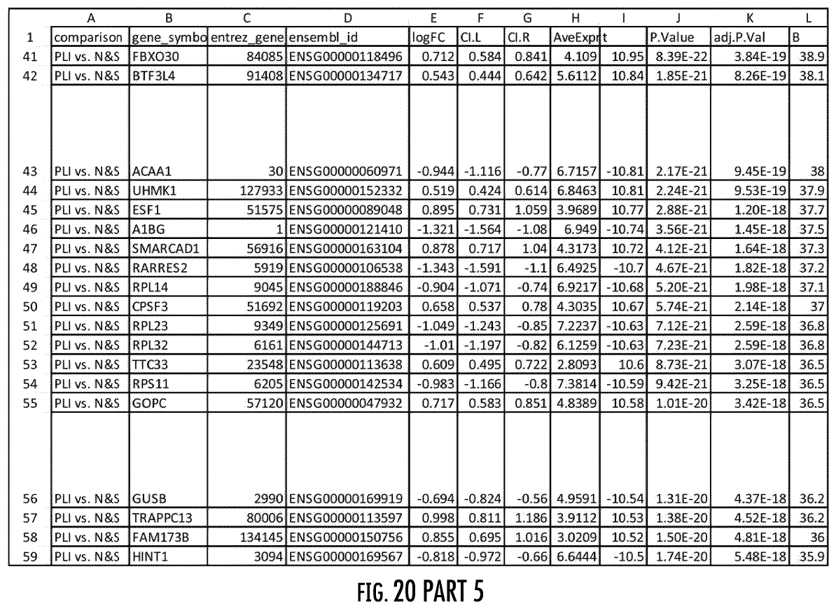

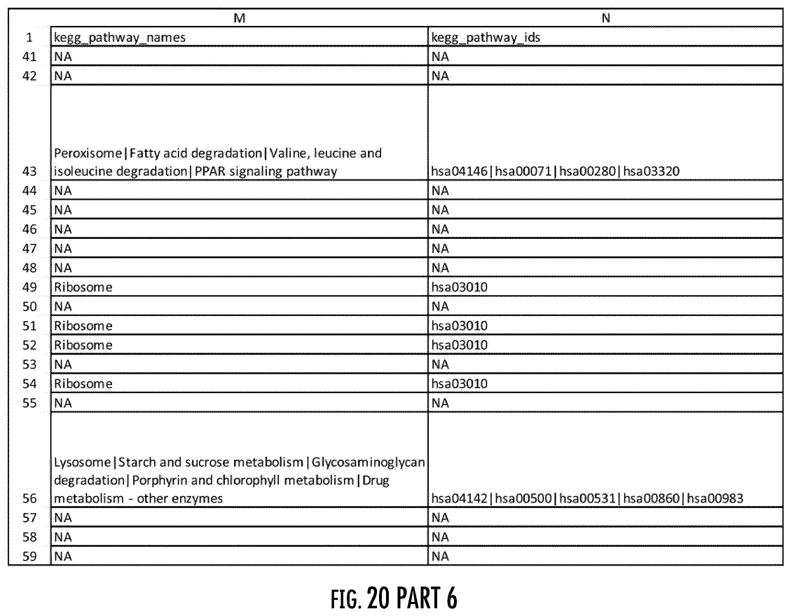

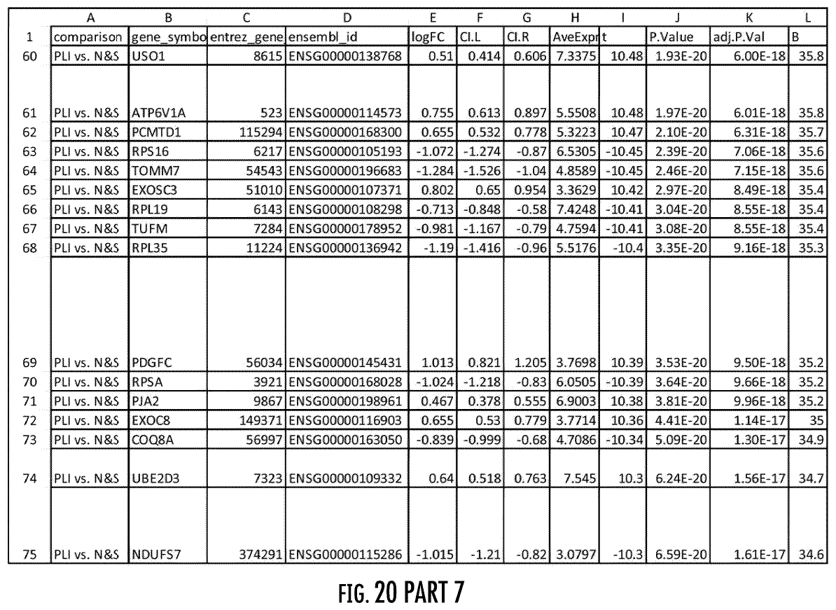

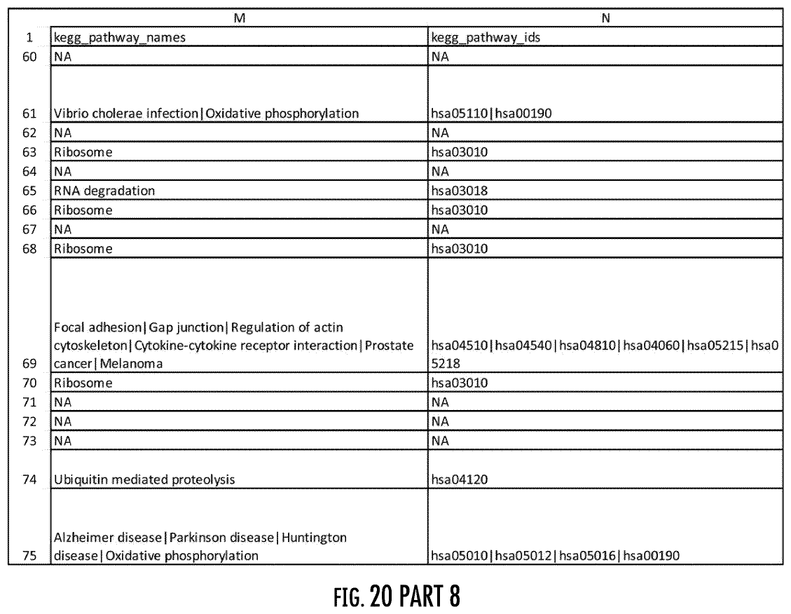

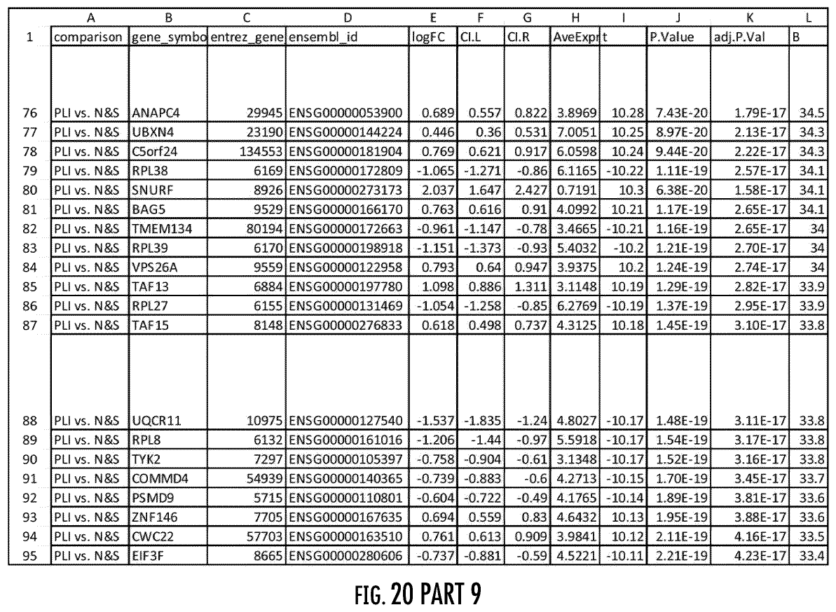

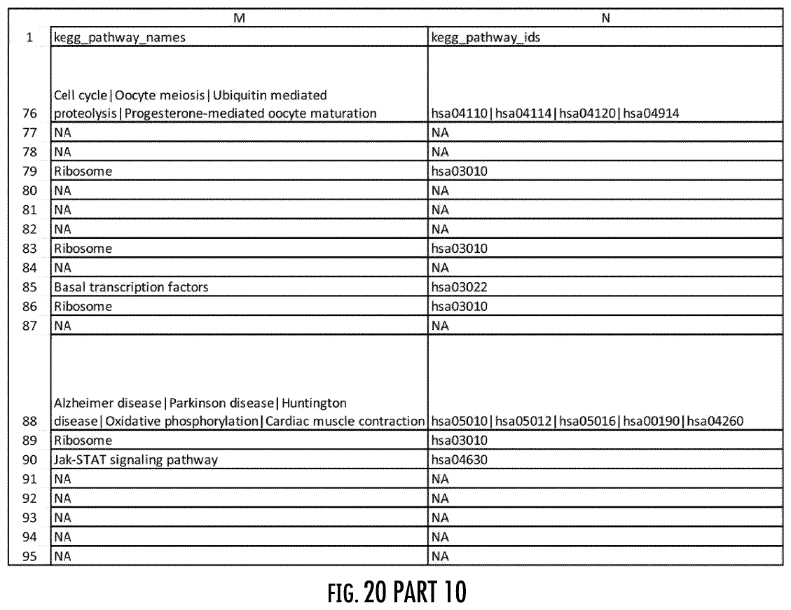

FIG. 20 is Data file S1. DEGs resulting from the 3 pairwise cluster comparisons.

These results were used in the creation of gene signatures (Data file S3) and NAFLD subnetwork (FIG. 19 and Table S6). The columns of this file are as follows:
The pairwise comparison
gene_symbol: Common gene name
Entrez gene ID
Ensembl gene ID
log FC: estimate of the log 2-fold-change of the comparison
CI.L: LogFC 95% confidence interval lower limit
CI.R: LogFC 95% confidence interval upper limit
AveExpr: average log 2-expression across all samples
t: moderated t-statistic (see Smyth (Smyth 2004))
P.Value: raw p-value
adj.P.Val: FDR corrected p-value
B: log-odds that the gene is differentially expressed
kegg_pathway_names: The names of the KEGG pathways that the gene is a member of (if applicable, NA otherwise)
kegg_pathway_ids: The pathway ids the KEGG pathways that the gene is a member of (if applicable, NA otherwise)

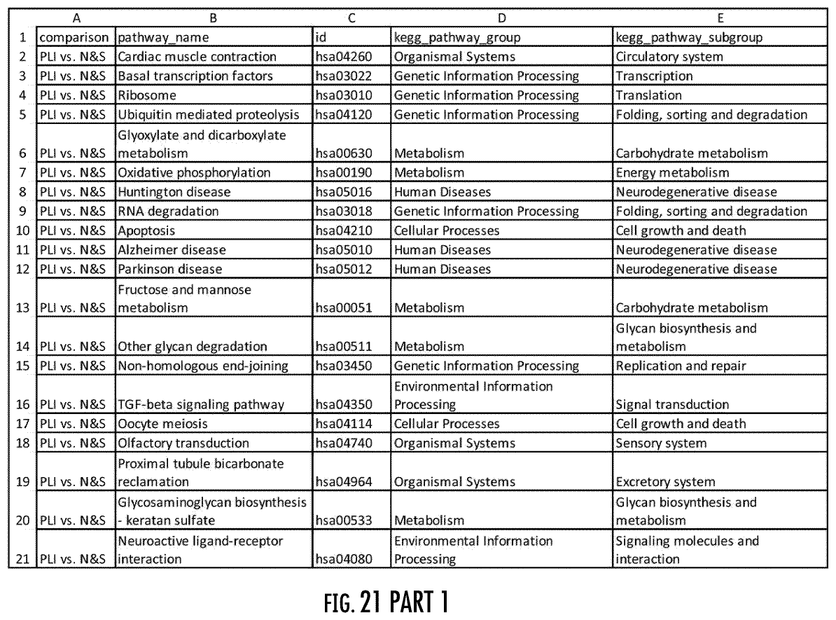

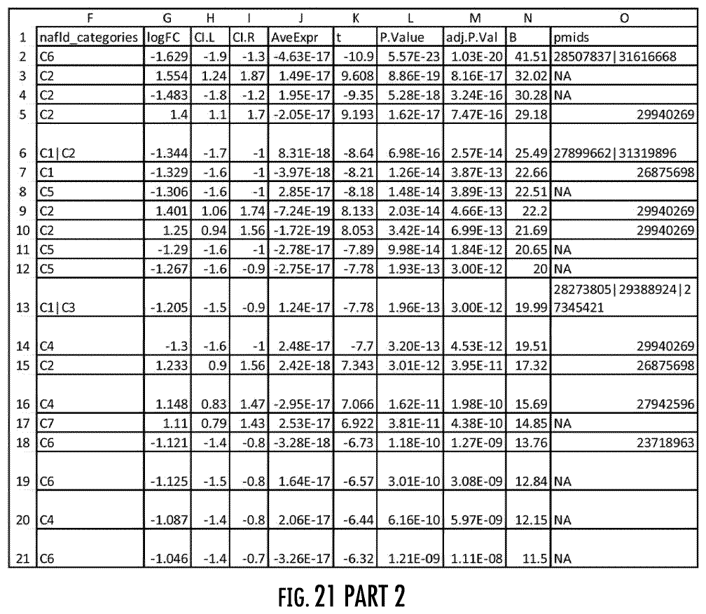

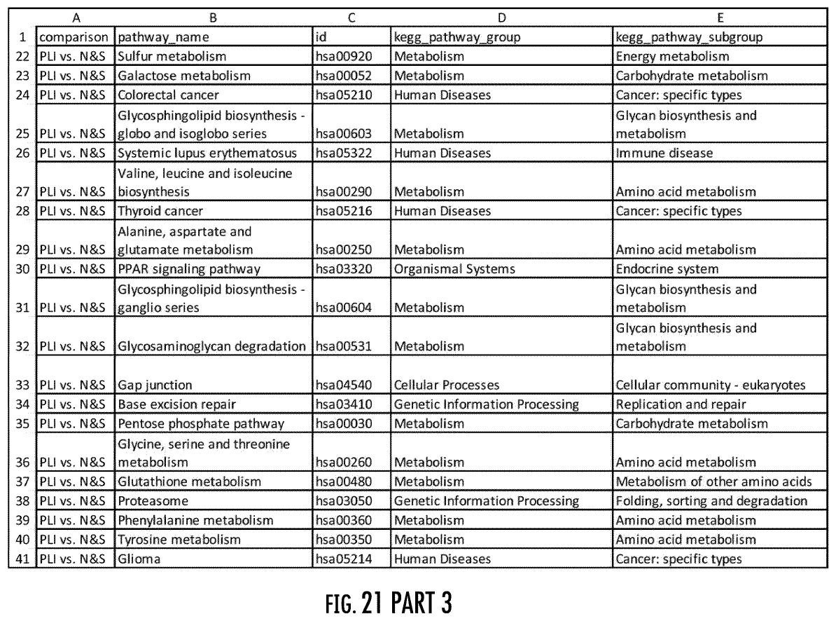

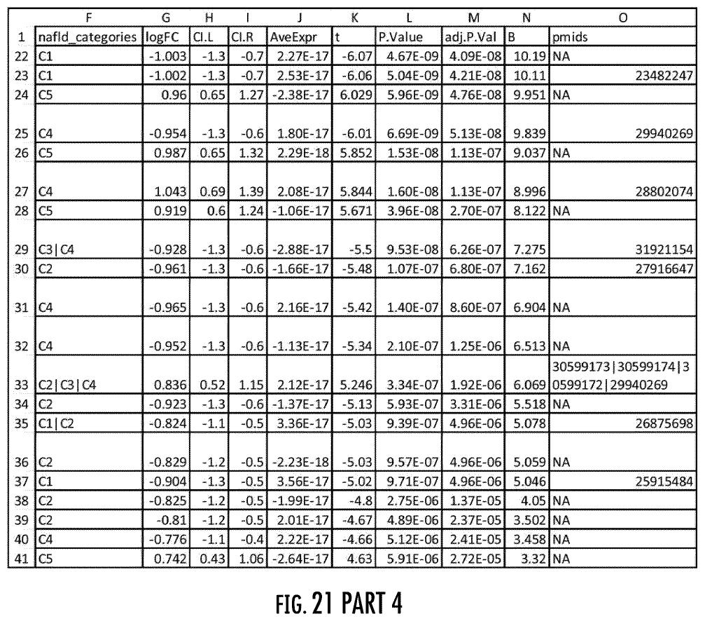

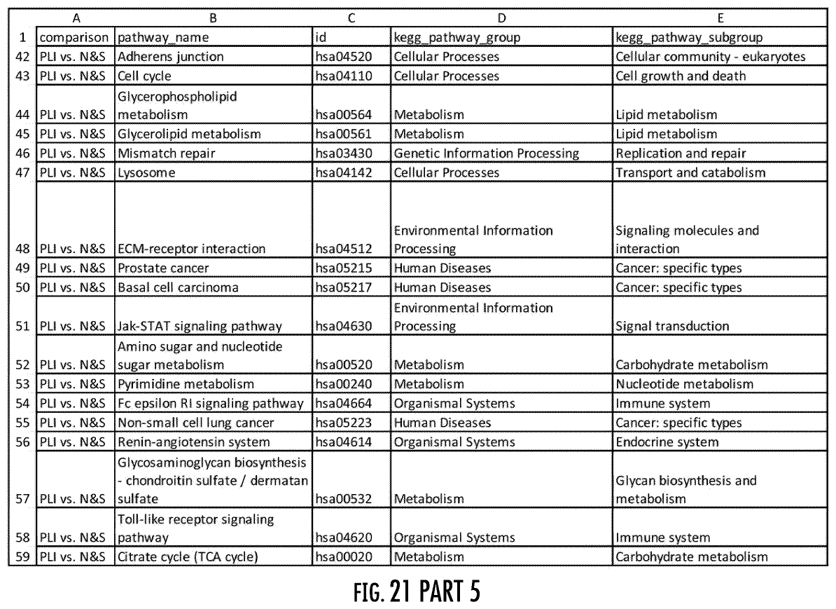

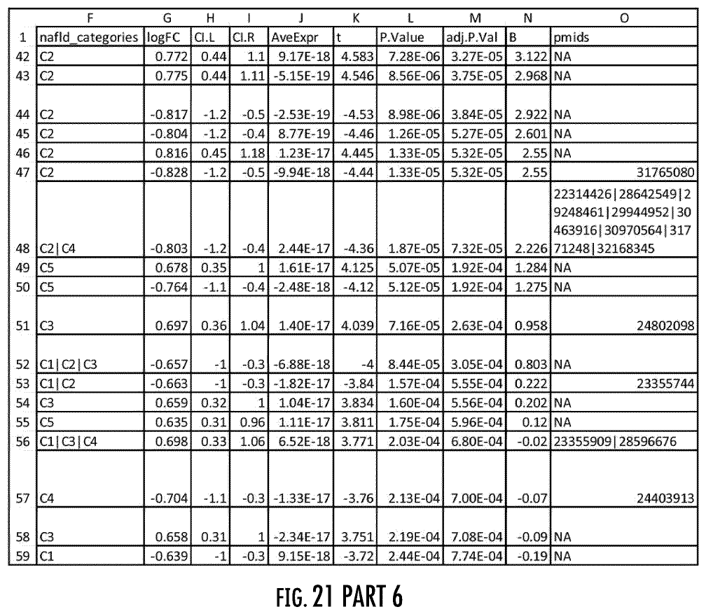

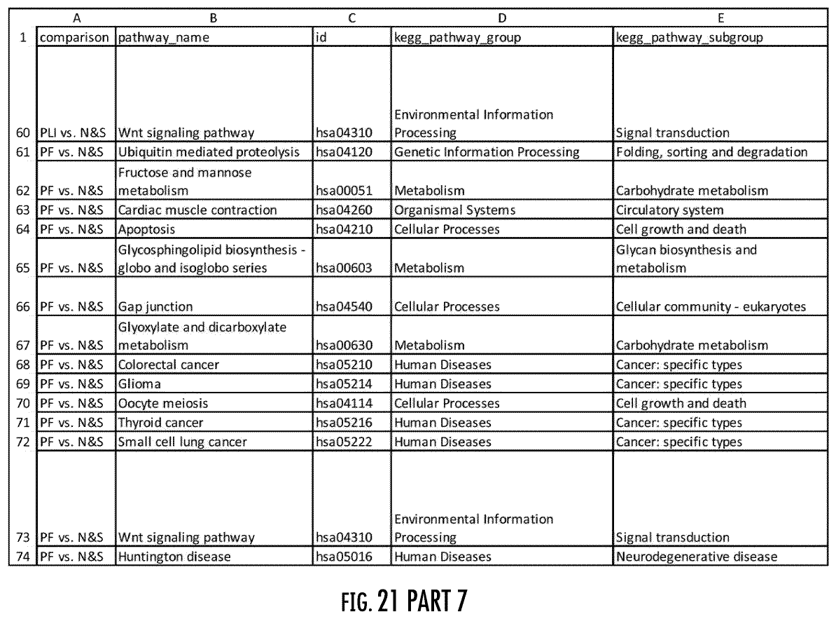

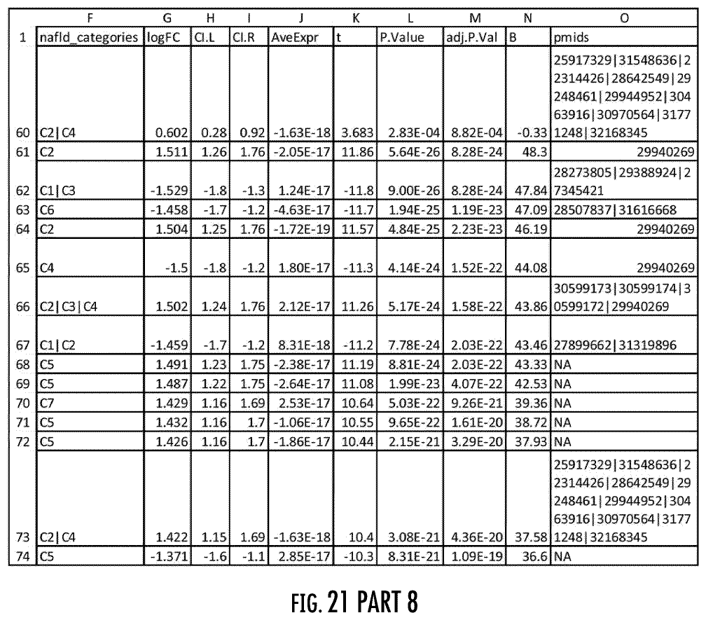

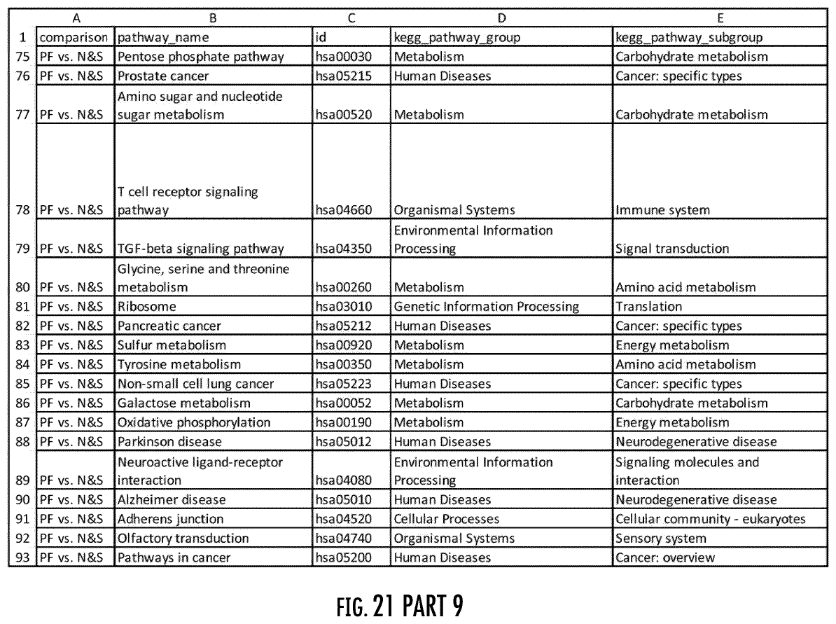

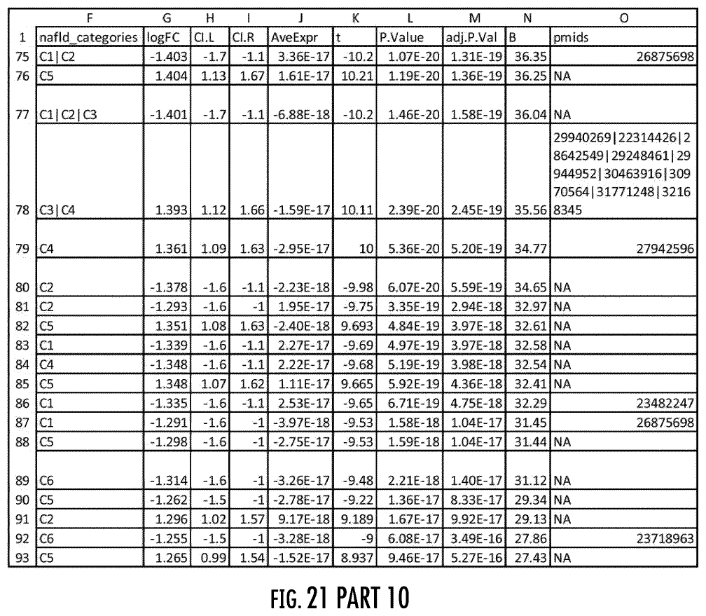

FIG. 21 is Data file S2. Differentially enriched pathways for each pairwise cluster comparison. These results were used to create Table S4, the gene signatures (Data file S3). See Example 1 and FIG. 12 for details. The columns of this file are as follows:
The pairwise comparison
Pathway name
id: KEGG pathway ID
KEGG pathway group
KEGG pathway subgroup
nafld_categories: Denotes the involvement of the pathway in NAFLD pathophysiology (see Methods).
   C1: Insulin resistance and oxidative stress
   C2: cell stress, apoptosis and lipotoxicity
   C3: Inflammation
   C4: Fibrosis
   C5: Disease related pathways
   C6: Other associated pathways
   C7: No established relationship
log FC: estimate of the log 2-fold-change of the comparison
CI.L: LogFC 95% confidence interval lower limit
CI.R: LogFC 95% confidence interval upper limit
AveExpr: average log 2-expression across all
t: moderated t-statistic (see Smyth (Smyth 2004))
P.Value: raw p-value
adj.P.Val: FDR corrected p-value
B: log-odds that the gene is differentially expressed
pmids: The PMIDs for the references which support the NAFLD categorization

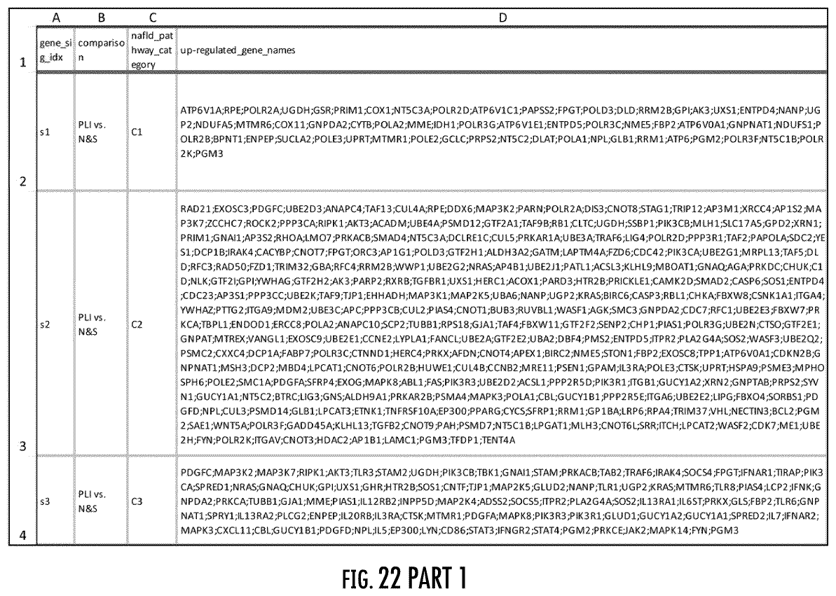

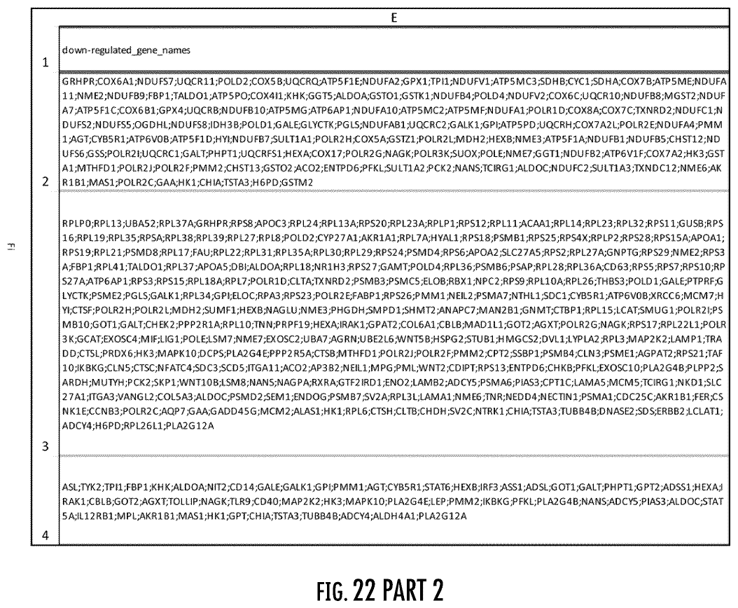

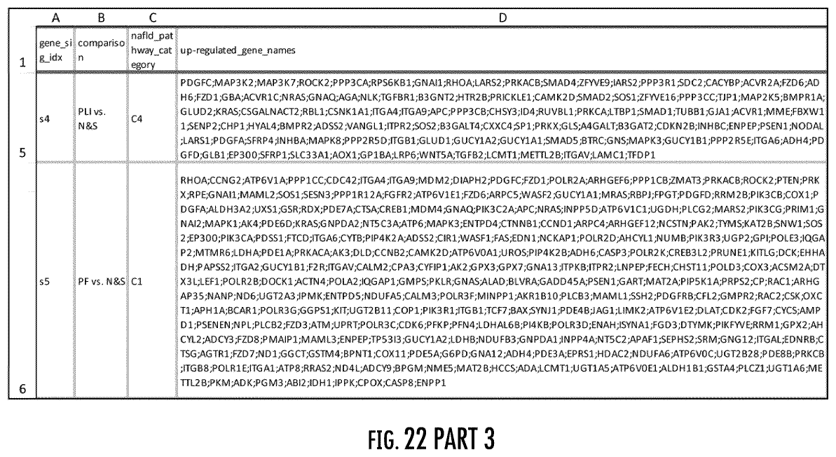

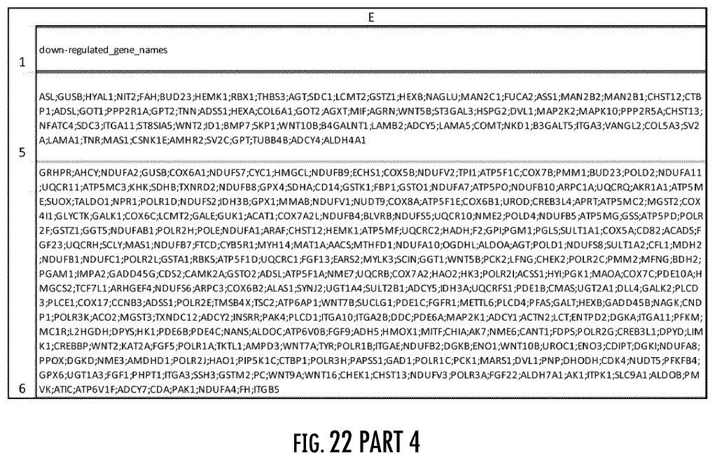

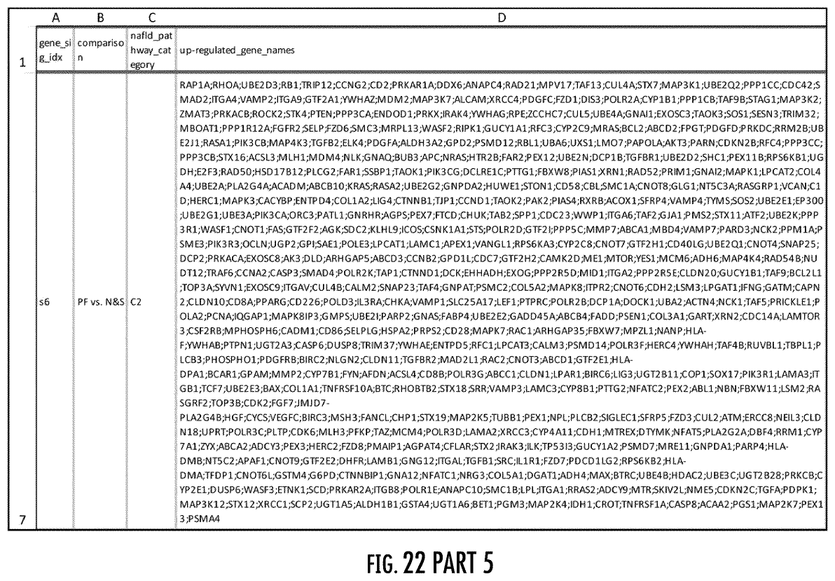

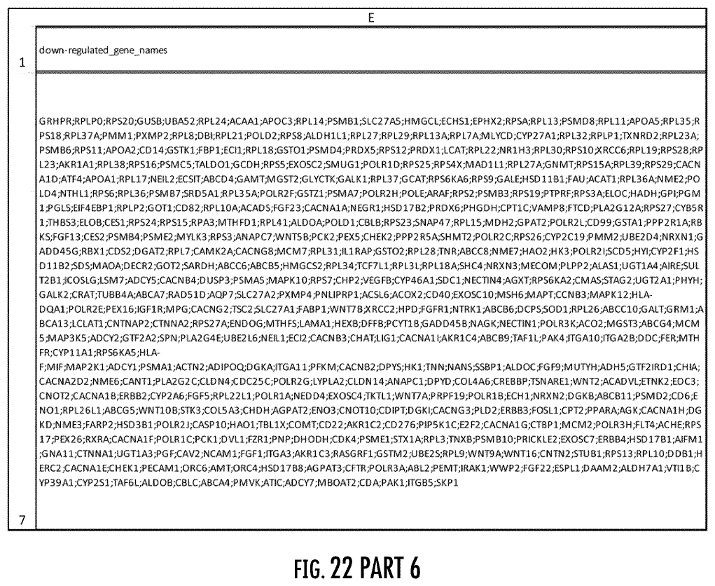

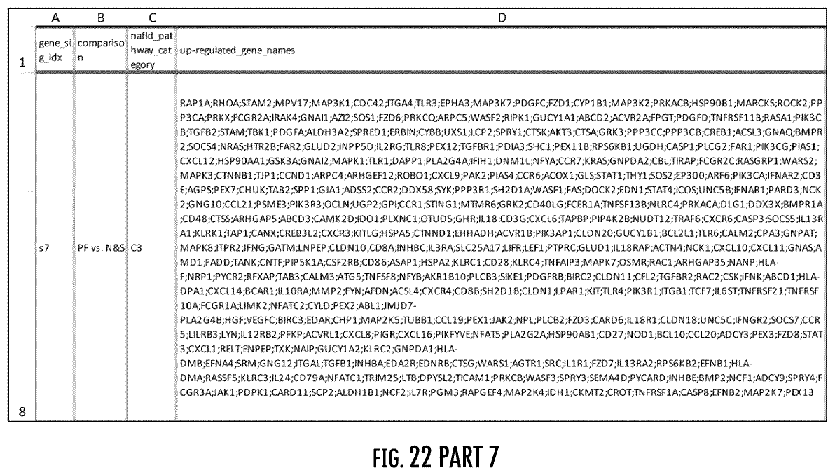

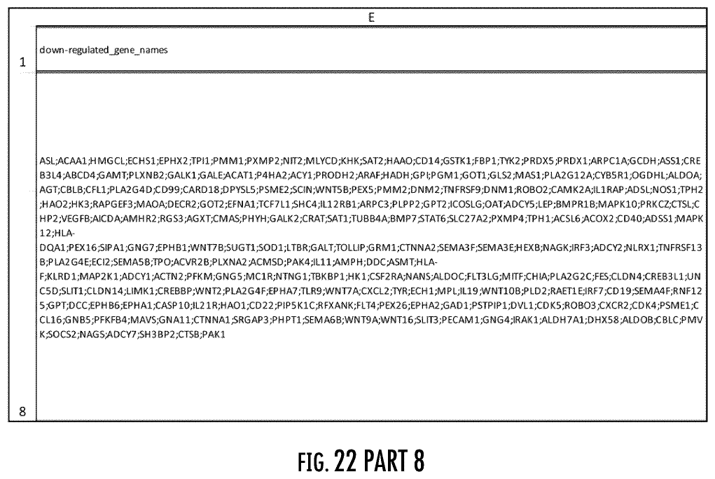

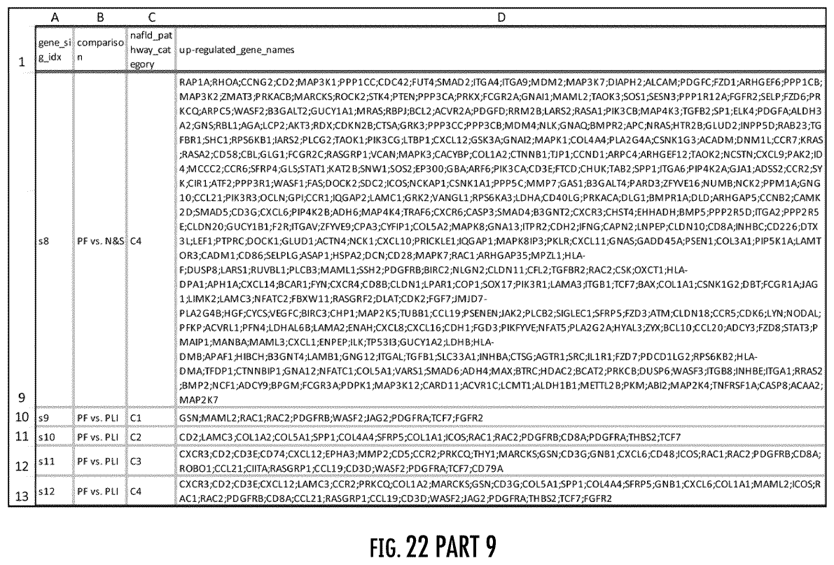

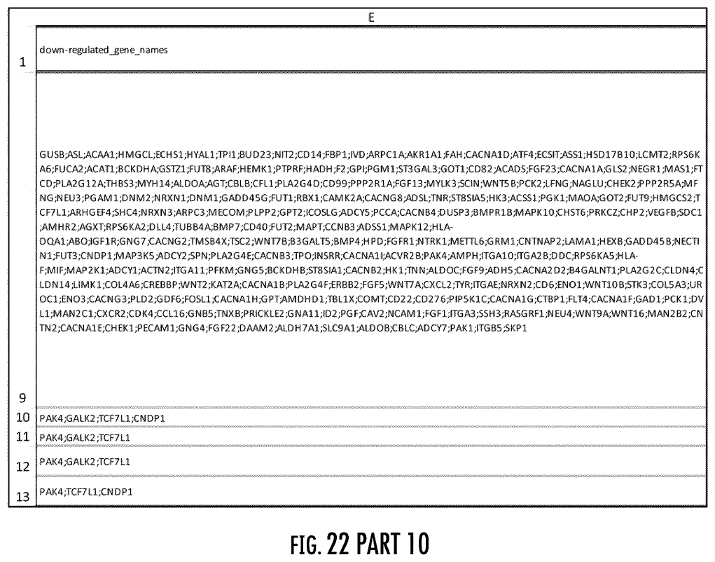

FIG. 22 is Data file S3. Gene signatures used for CMap analysis.

The data from Data files S1 & S2 were used to create this file (see Example 1; FIG. 18). It was used for CMap drug prediction (Table S5; Data file S4, see FIG. 18; Methods for details on the methodology). The columns are as follows:
gene_sig_idx: The gene signature index (see Table S3 and Data file S3)
The pairwise comparison
nafld_pathway_category: The NAFLD category of differentially enriched pathways that was used to create the gene signature (see Methods; FIG. 15), The values are defined as follows:
   C1: Insulin resistance and oxidative stress
   C2: cell stress, apoptosis and lipotoxicity
   C3: Inflammation
   C4: Fibrosis
up-regulated_gene_names: List of the upregulated genes (using common gene name) for the signature
up-regulated_entrez_ids: List of the upregulated genes (using entrez gene id) for the signature
down-regulated_gene_names: List of the down-regulated genes (using common gene name) for the signature
down-regulated_entrez_ids: List of the down-regulated genes (using entrez gene id) for the signature

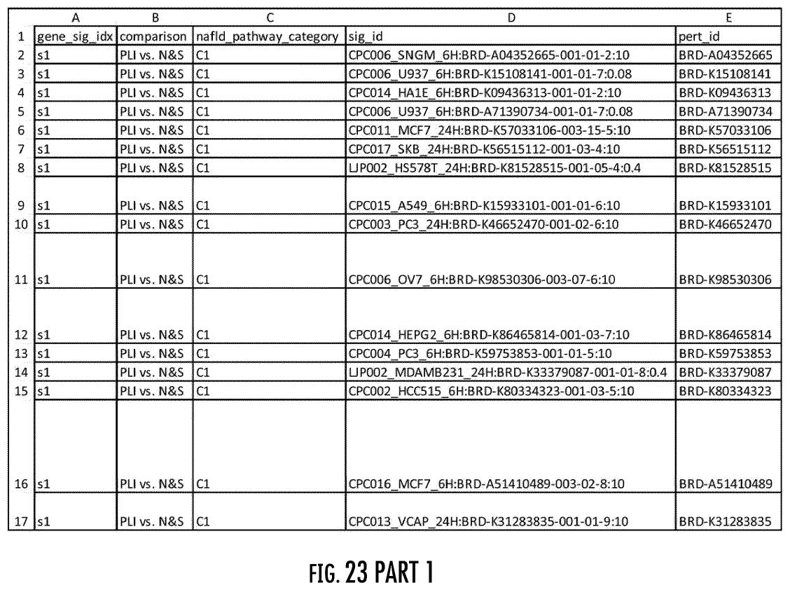

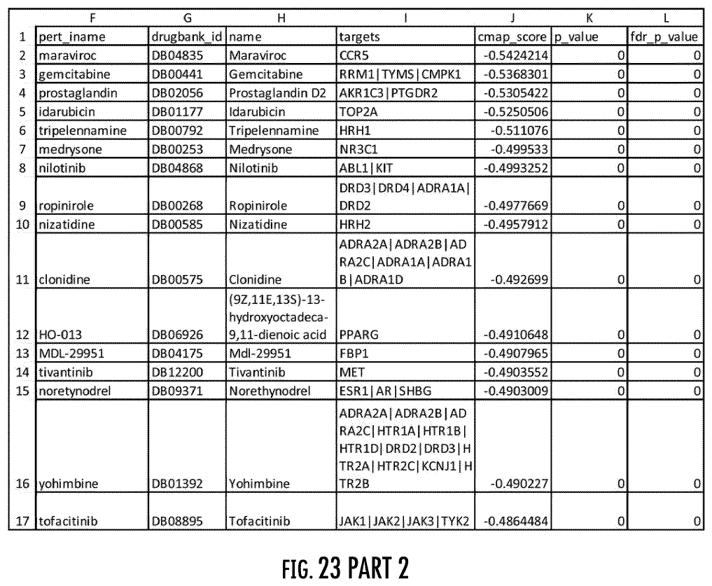

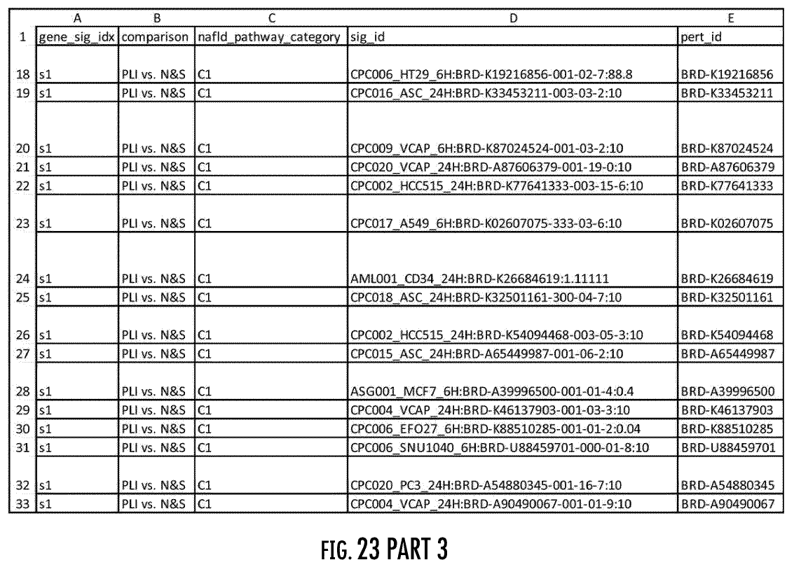

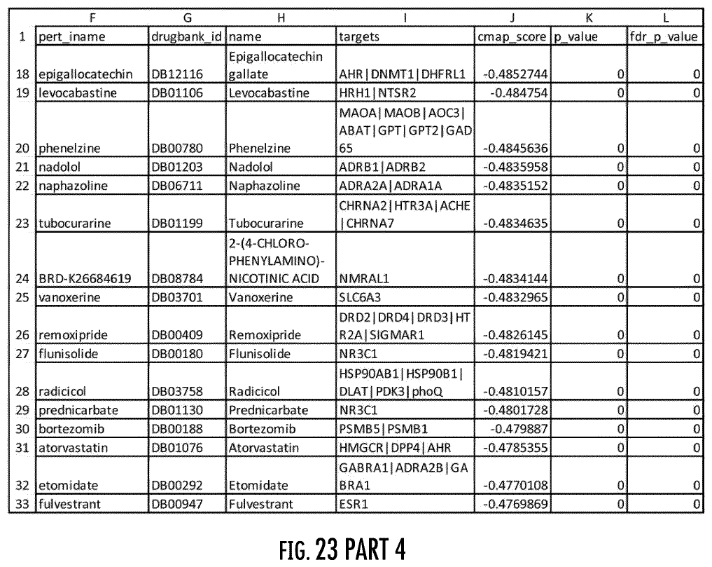

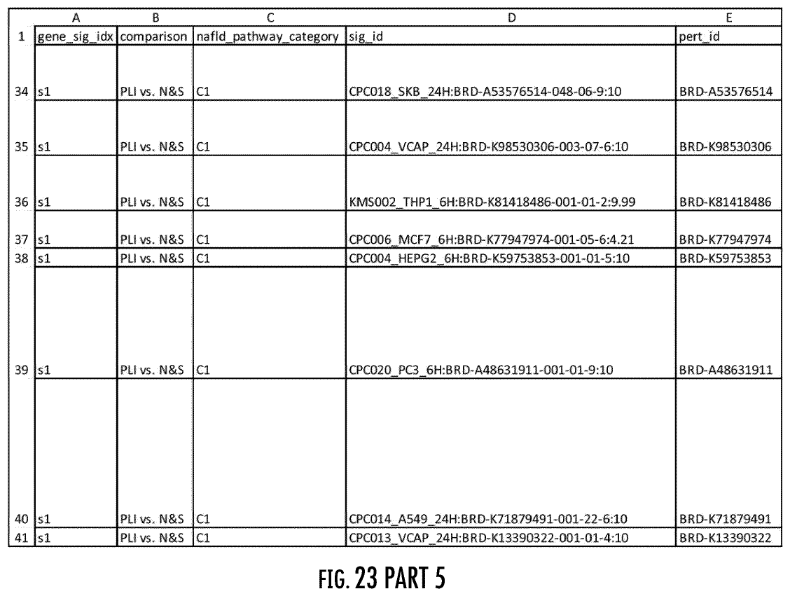

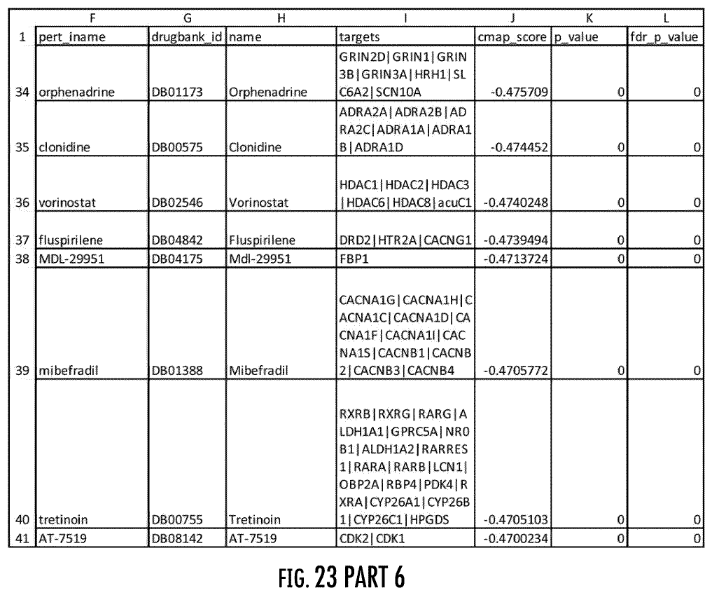

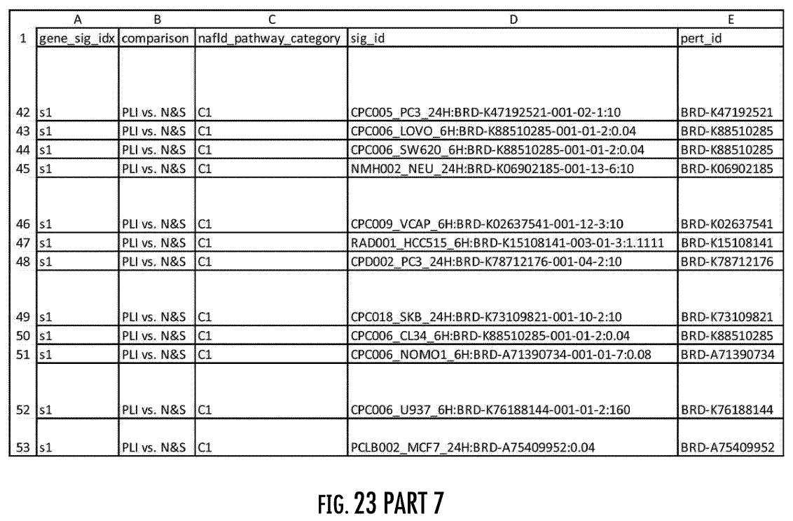

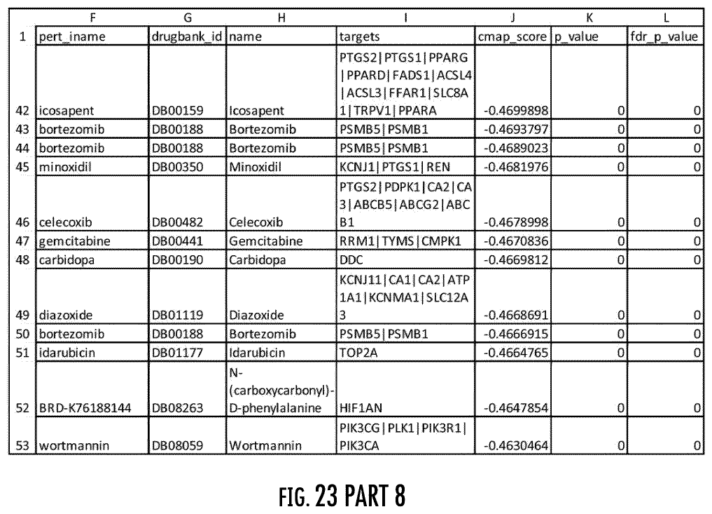

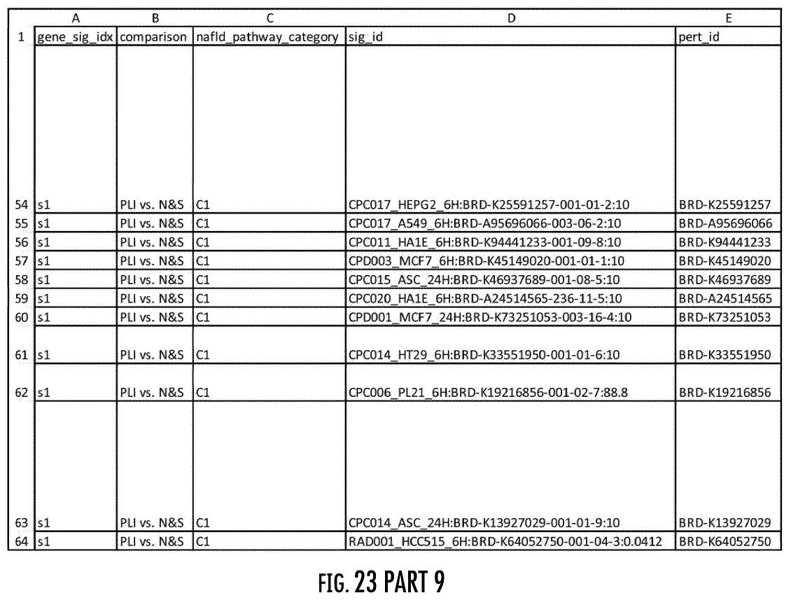

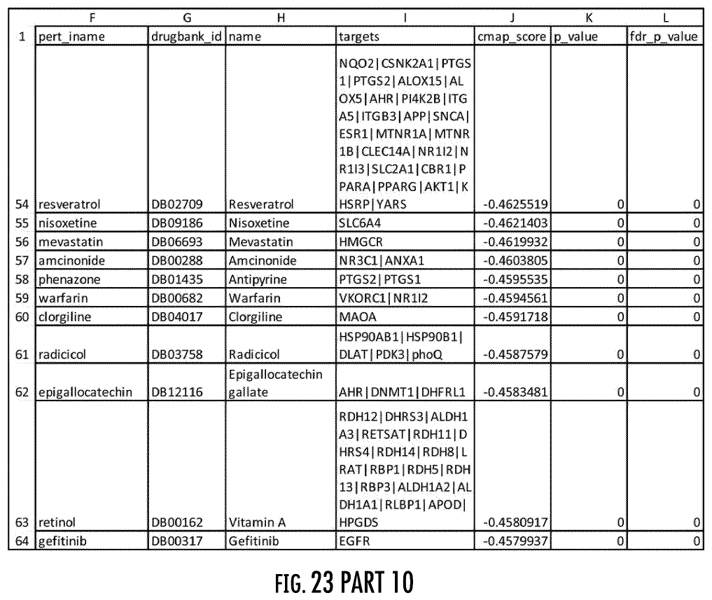

FIG. 23 is Data file S4. Non-zero CMAP scores of small molecules with a DrugBank ID for the 12 queries described in FIG. 18 and Methods. These results were used to create Table 1 & S5 (see Example 1 and FIG. 18 for details). The file is 292,246 rows by 10 columns. The columns are as follows:
gene_sig_idx: The gene signature index (see Table S3 and Data file S3)
The pairwise comparison
nafld_pathway_category: The NAFLD category of differentially enriched pathways that was used to create the gene signature (see Methods; FIG. 18), The values are defined as follows:
   C1: Insulin resistance and oxidative stress
   C2: cell stress, apoptosis and lipotoxicity
   C3: Inflammation
   C4: Fibrosis
sig_id: The L100 perturbation instance signature id
pert_id: The Broad's internal drug/small molecule ID
pert_iname: The Broad's drug/small molecule common name
drugbank_id: DrugBrank's drug/small molecule ID
name: DrugBrank's drug/small molecule common name
targets: The drug/small molecule targets from DrugBank v5.1.4
cmap_score: The CMap score (see Example 1; FIG. 15, and (Lamb, Crawford et al. 2006, Subramanian, Narayan et al. 2017))
p_value: P-value calculated by permutation testing (see Chen et al (Chen, Wei et al. 2017))
fdr_p_value: False discovery rate corrected p-value

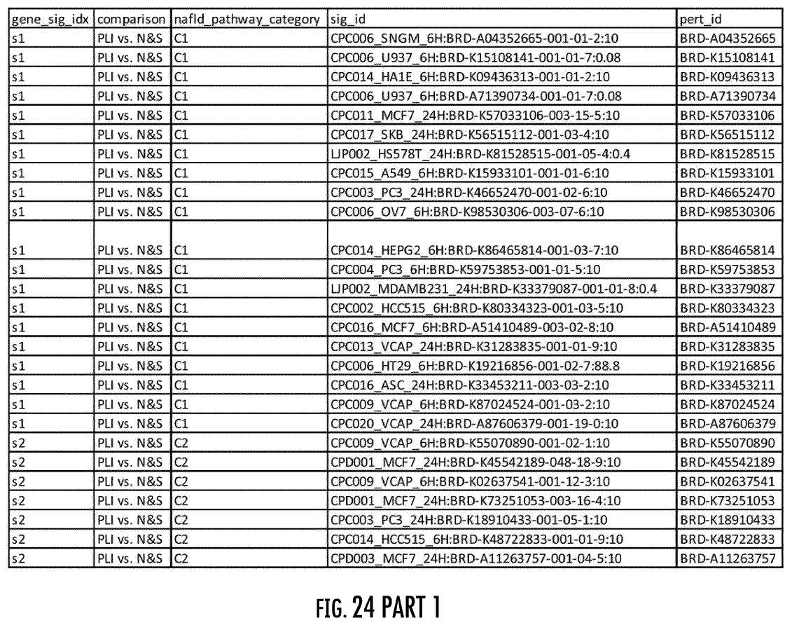

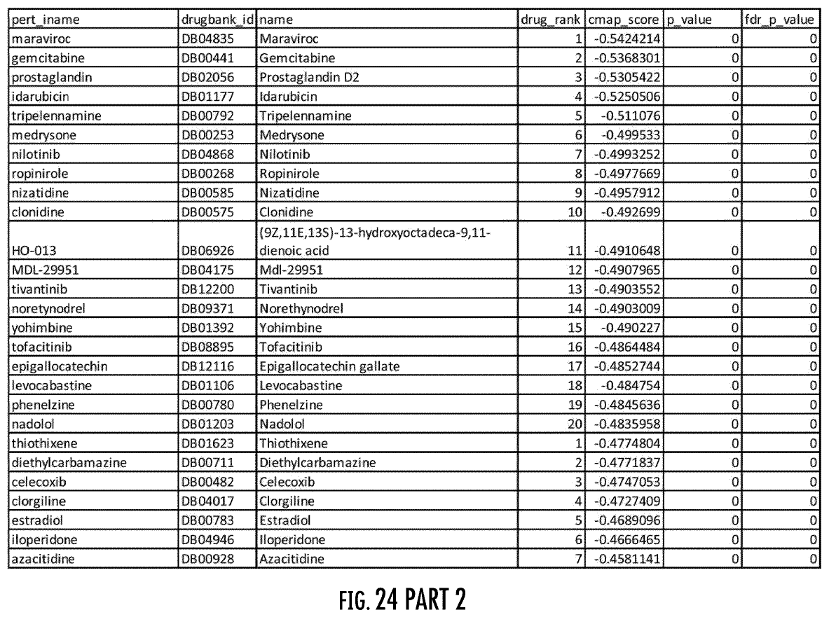

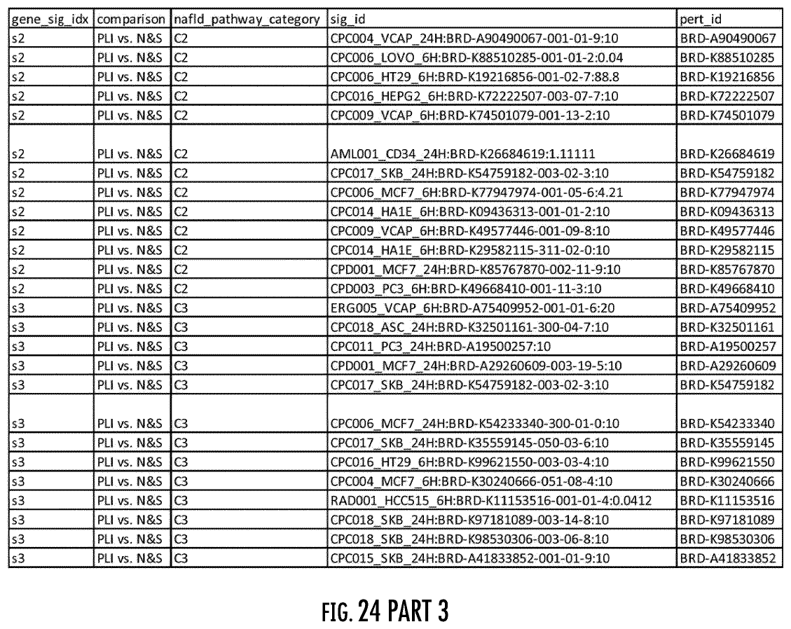

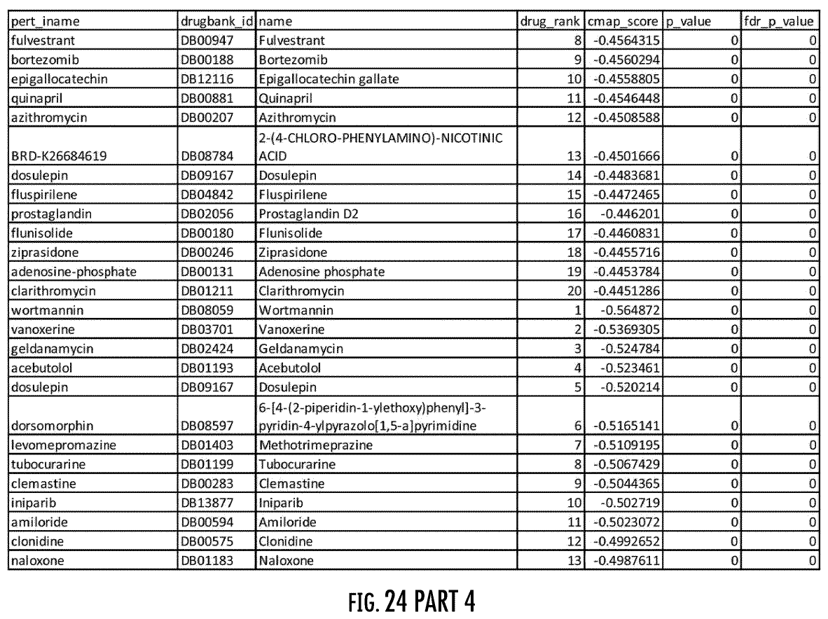

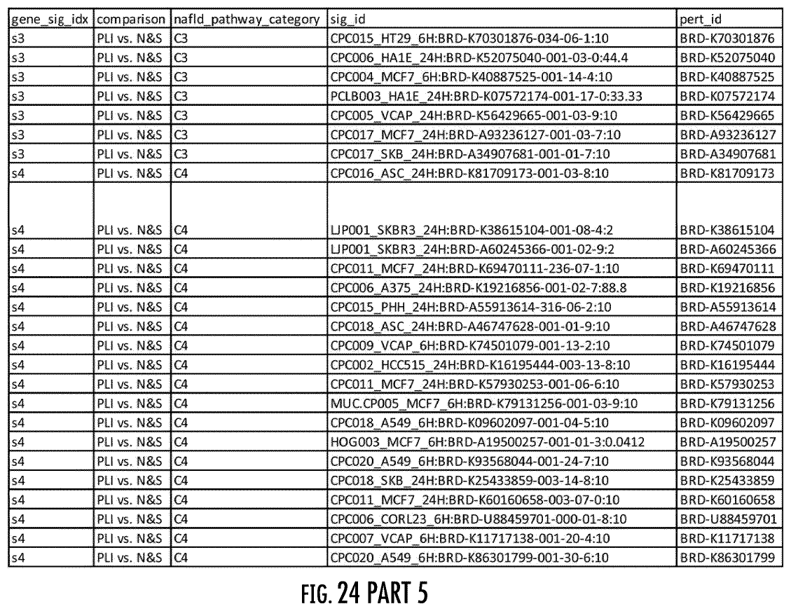

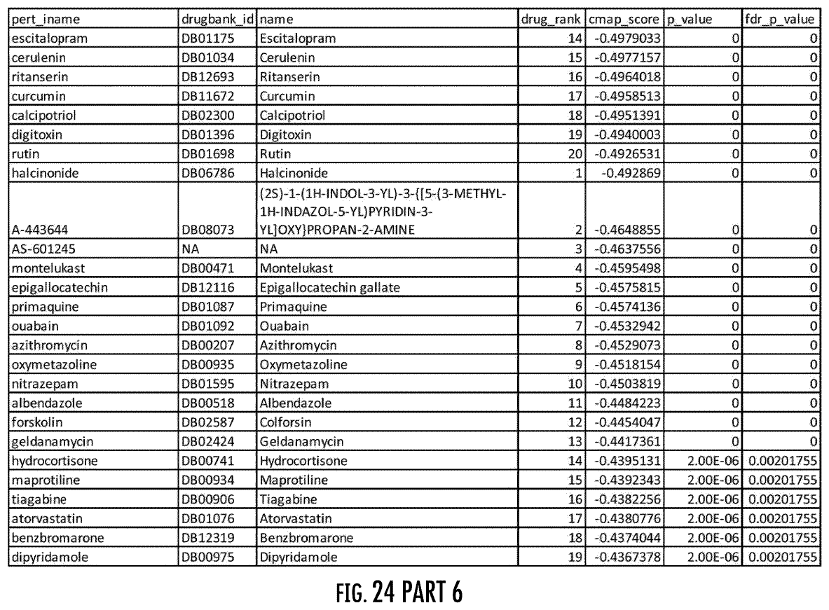

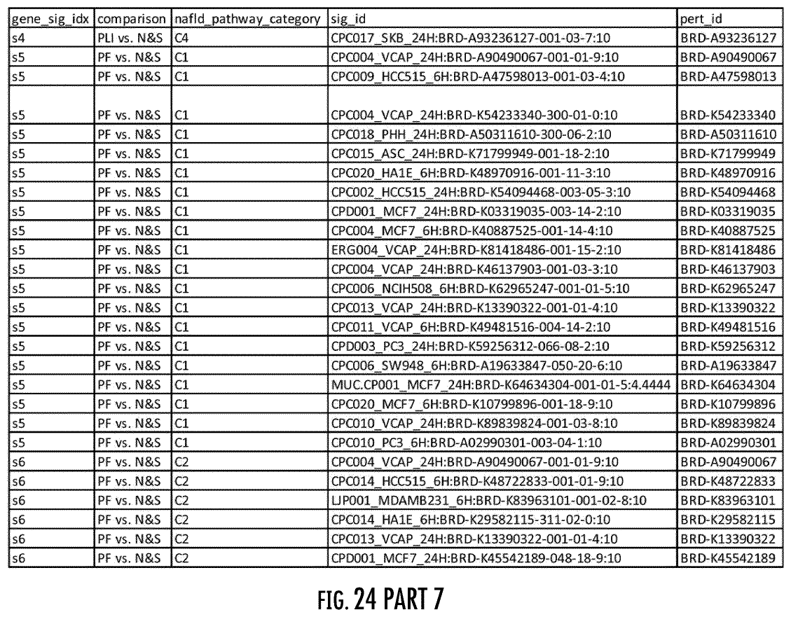

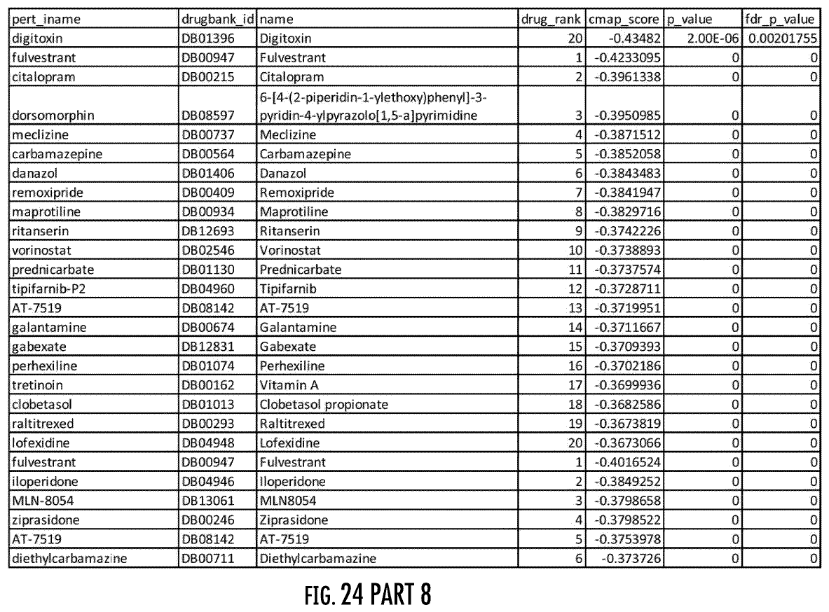

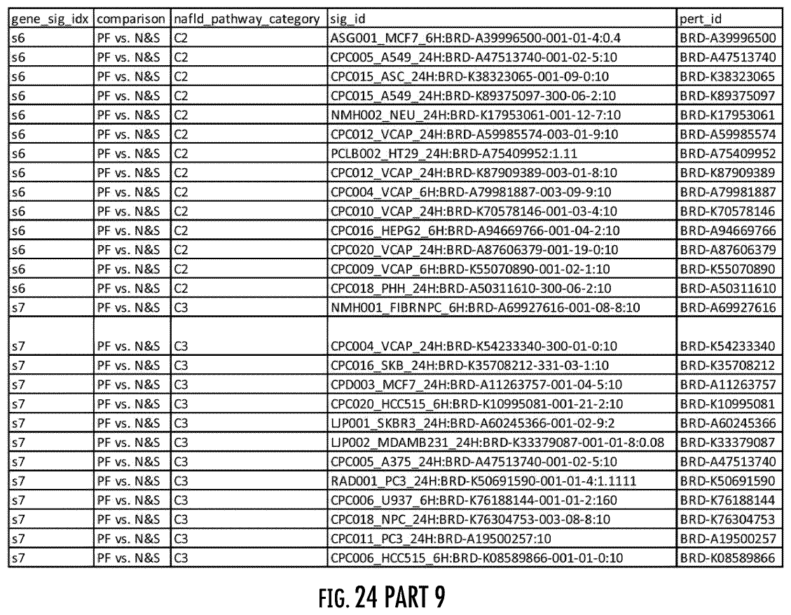

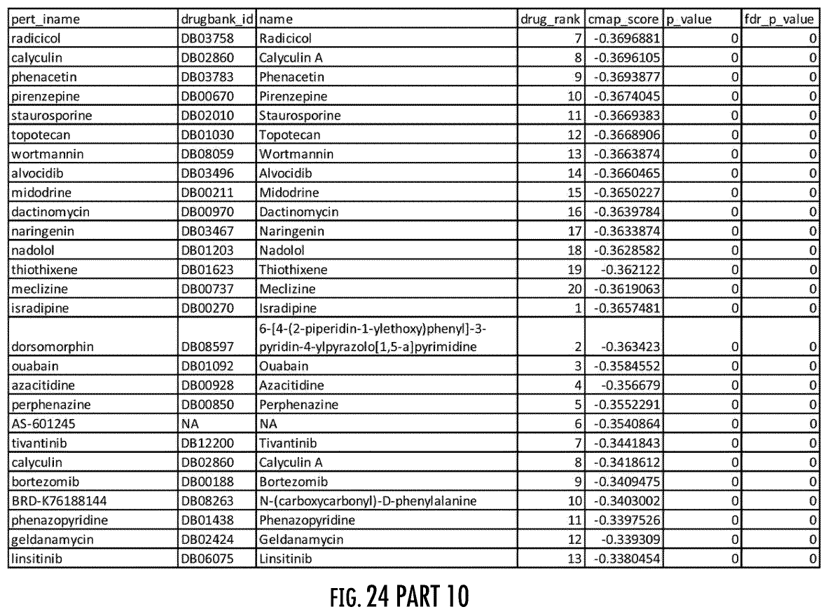

FIG. 24 is Data file S5. List of top 20 CMap (FDR p-value<0.05) predictions from the 12 signatures (196 predictions, 139 unique compounds). This file contains the top 20 CMap predictions (FDR p-value<0.05) from each of the 12 gene signatures. These results were created from Data file S4 and were used to create Table 1 and Table S5. The columns are as follows:
gene_sig_idx: The gene signature index (see Table S3 and Data file S3)
The pairwise comparison
nafld_pathway_category: The NAFLD category of differentially enriched pathways that was used to create the gene signature (see Example 1; FIG. 15), The values are defined as follows:
   C1: Insulin resistance and oxidative stress
   C2: cell stress, apoptosis and lipotoxicity
   C3: Inflammation
   C4: Fibrosis
sig_id: The L100 perturbation instance signature id
pert_id: The Broad's internal drug/small molecule ID
pert_iname: The Broad's drug/small molecule common name
drugbank_id: DrugBrank's drug/small molecule ID
name: DrugBrank's drug/small molecule common name
drug_rank: Relative rank of the compound prediction within the gene signature
cmap_score: The CMap score (see Example 1; FIG. 15, and (Lamb, Crawford et al. 2006, Subramanian, Narayan et al. 2017))
p_value: P-value calculated by permutation testing (see Chen et al (Chen, Wei et al. 2017))
fdr_p_value: False discovery rate corrected p-value

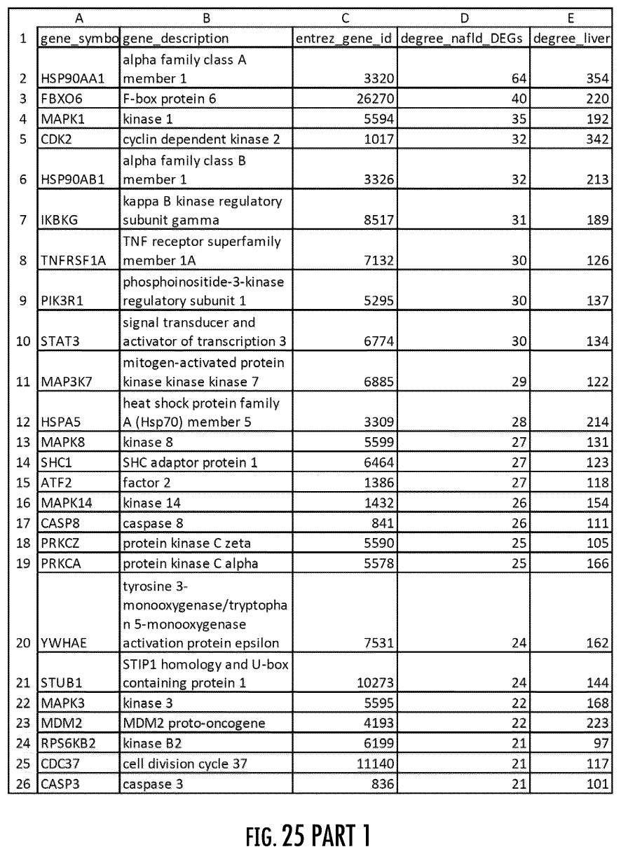

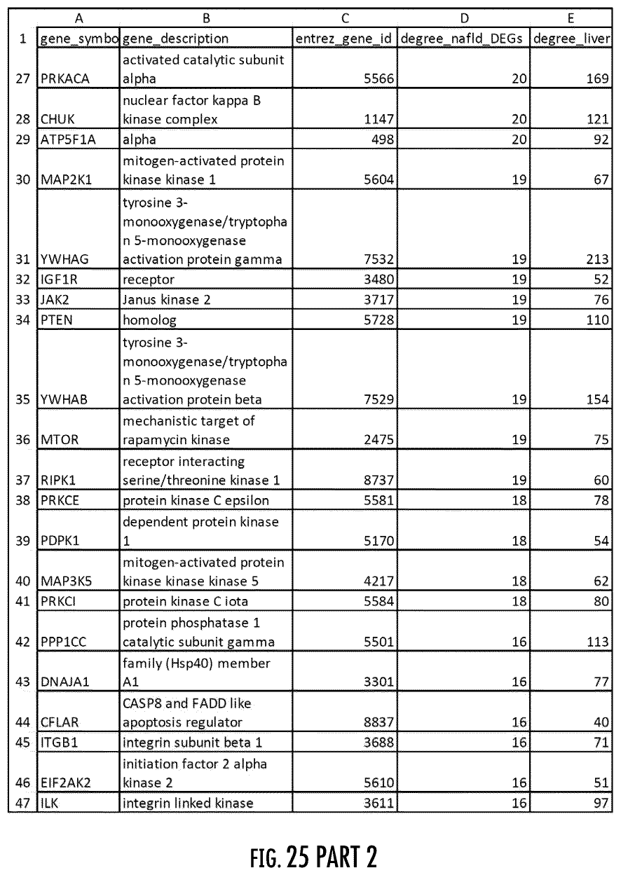

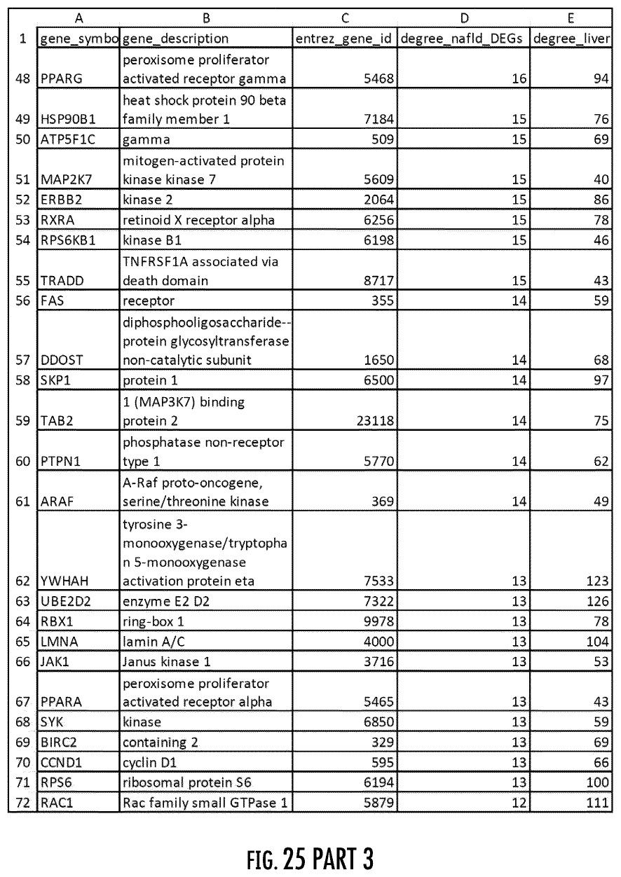

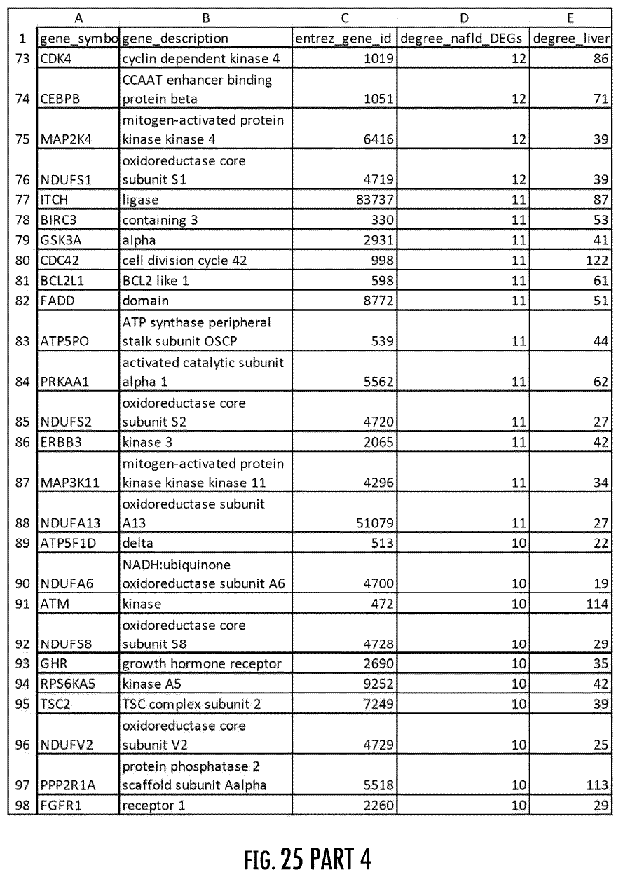

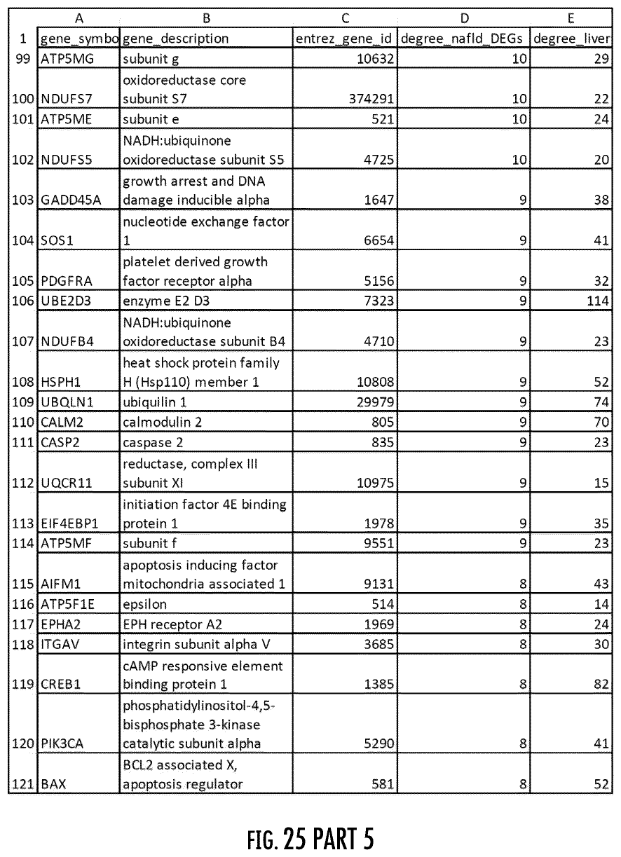

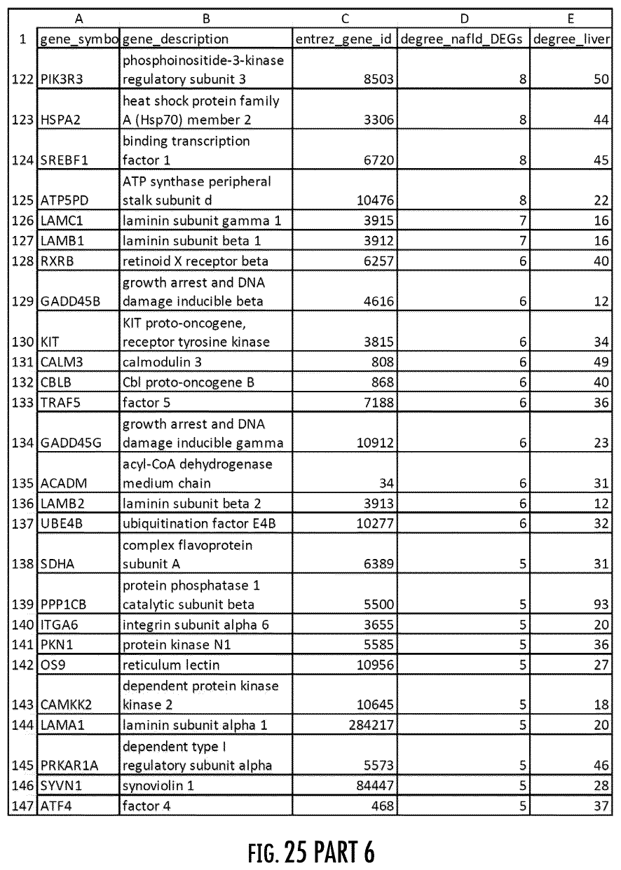

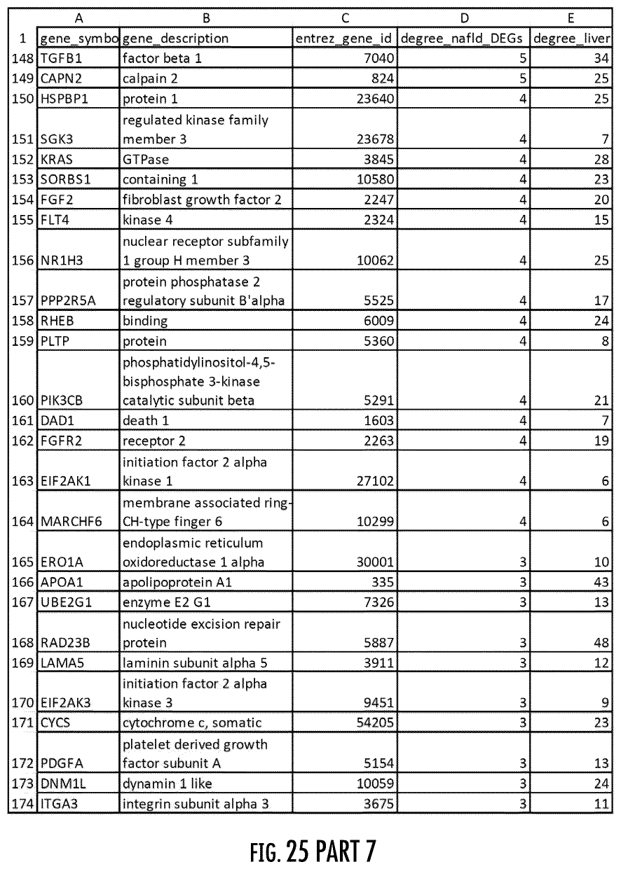

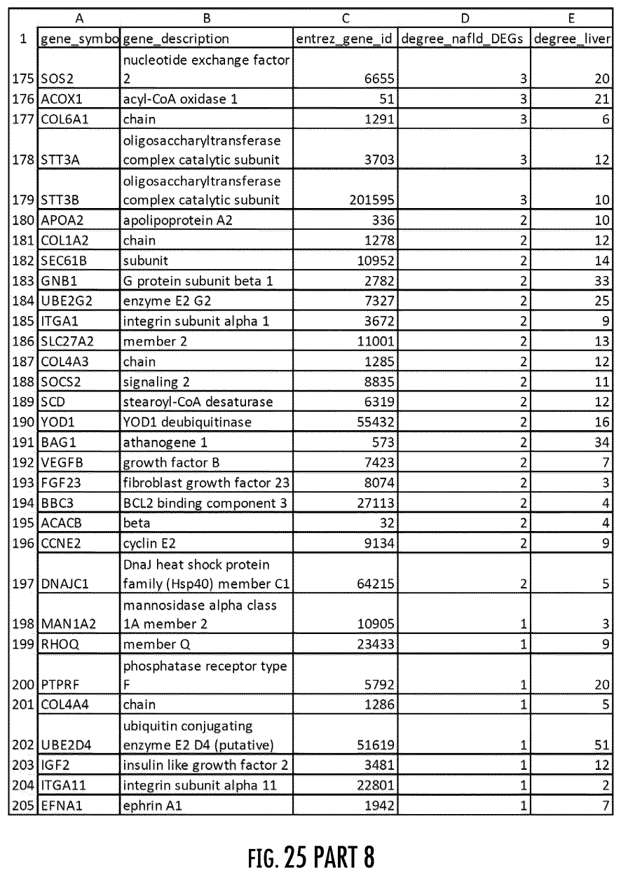

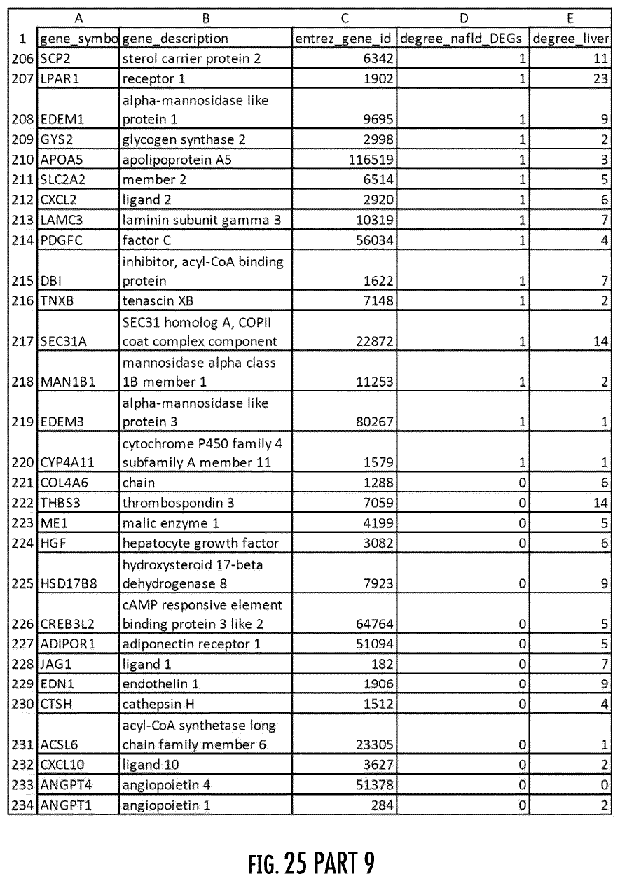

FIG. 25 is Data file S6. Degree of the nodes in the NAFLD subnetwork. These results are discussed in the results section of the main text and supports Table S6. The columns are as follows:
The common gene name or symbol
Gene description
Entrez gene id
degree_liver: The number of connections this protein has to other nodes in the human liver interactome
degree_nafld_DEGs: The number of connections the encoded protein has with other DEG encoded nodes in the NAFLD associated network

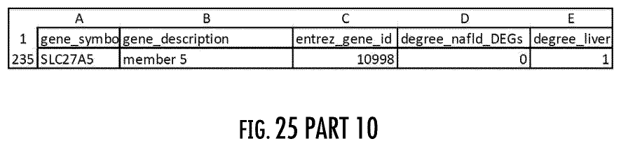

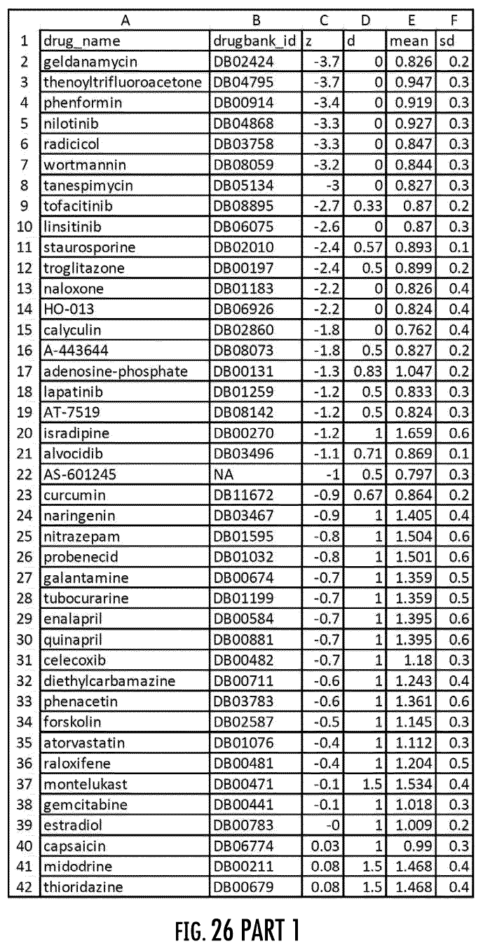

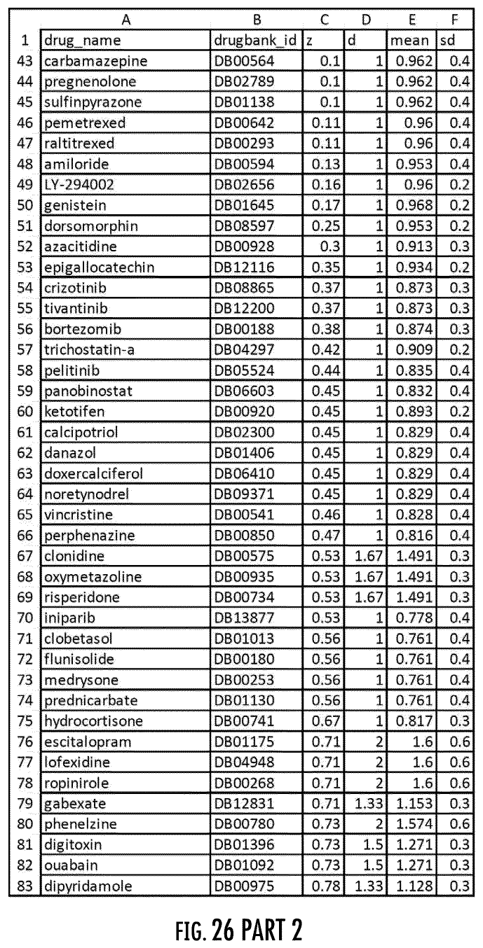

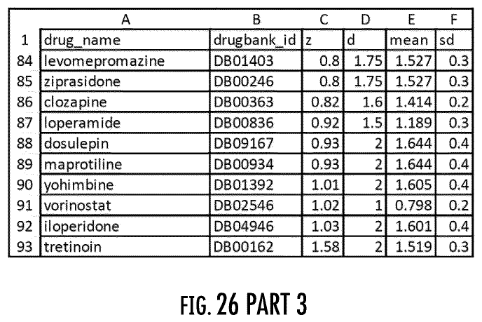

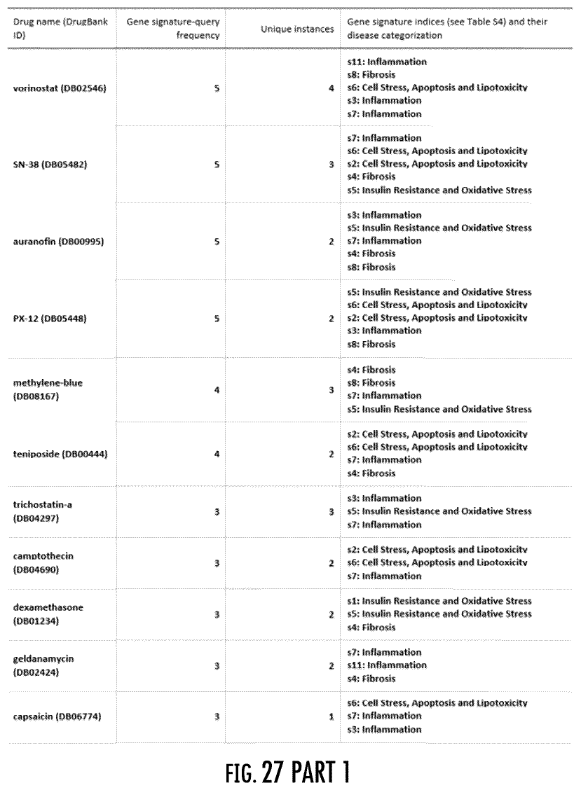

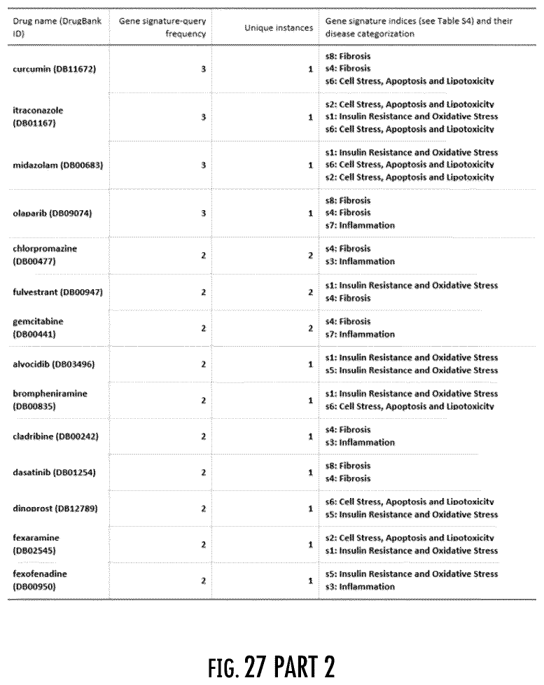

FIG. 26 is Data file S7. Network proximity determined Z-scores for the highest ranking CMap-predicted drugs with targets mapping to the NAFLD subnetwork. These results were used for Table 1, FIG. 7, and Table S6 & S7. The columns are as follows:
Common name of the drug/small molecule
drugbank_id: DrugBank ID of the drug/small molecule
Common gene name
Entrez gene ID
z: Z-score of the normalized distance of drug subnetwork to disease associated subnetwork (See Example 1, (Guney, Menche et al. 2016))
d: Shortest distance of drug subnetwork to disease associated subnetwork (See Example 1, (Guney, Menche et al. 2016))
mean: Average distance of a reference network to disease associated subnetwork (See Example 1, (Guney, Menche et al. 2016))
sd: Standard deviation of a reference network to disease associated subnetwork (See Example 1, (Guney, Menche et al. 2016))

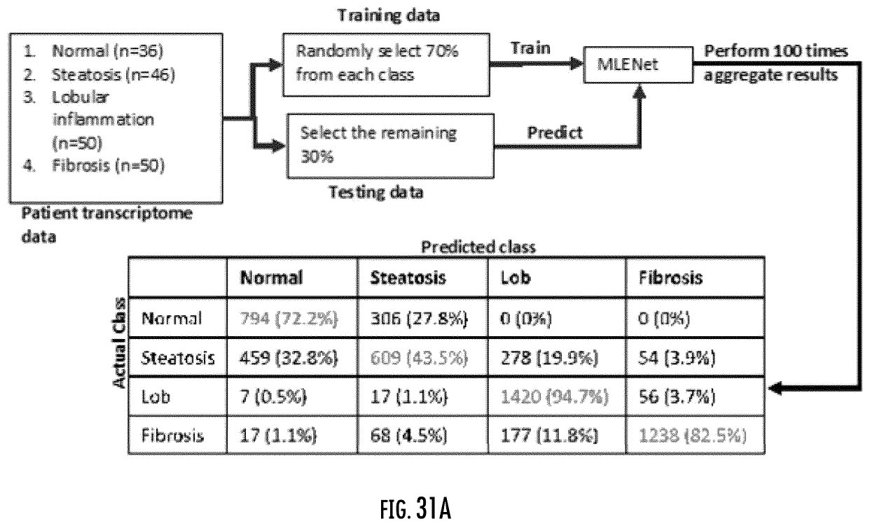

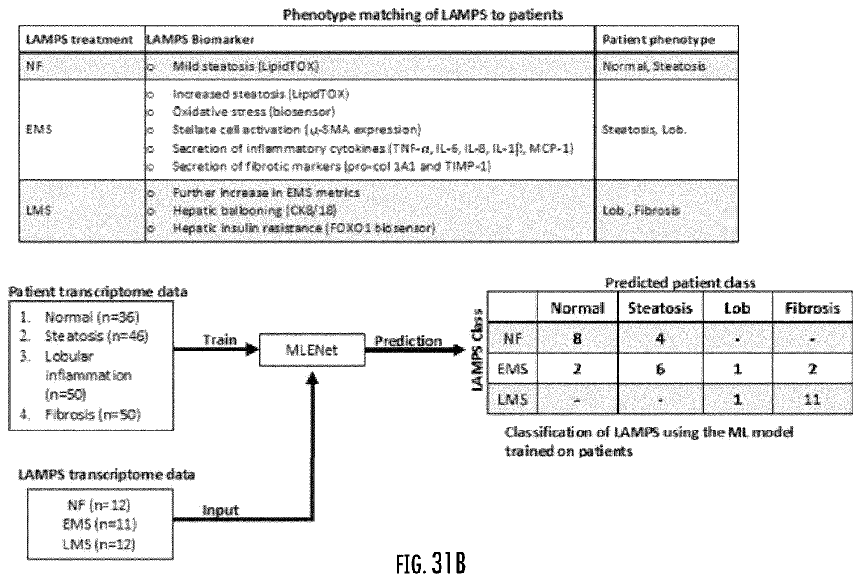

FIG. 31A-31B illustrate unbiased machine learning model of patient transcriptomic data identifies and predicts congruent clinical phenotypes within LAMPS. FIG. 31A shows the bootstrapping procedure used to develop and validate the transcriptome-based machine learning model (MLENet) capable of differentiating and predicting 4 NAFLD patient classifications (see Methods) (red indicates the clinically defined true positives). The average sensitivity across the bootstrapping instances (numbers in parenthesis are standard deviations) are: 0.66 (0.11), 0.64 (0.12), 0.77 (0.08), 0.93 (0.07); average specificity 0.93 (0.03), 0.83 (0.03), 0.98 (0.02), 0.95 (0.03) for Normal, Steatosis, Lob, and Fibrosis respectively. FIG. 31 B shows the workflow and table of outcomes from implementing MLENet to identify and predict congruent NAFLD patient phenotypes from LAMPS transcriptomic analytes generated under normal fasting (NF); early metabolic syndrome (EMS); or late metabolic syndrome (LMS) conditions (see Methods). The phenotype matching of LAMPS to patients results from extensive parallel biochemical and imaging analyses [Saydmohammed, M., et al. 2021] indicating that the three different media conditions drive distinct phenotypes congruent with clinical phenotypes of NAFLD progression and are independently consistent with the machine learning approach.

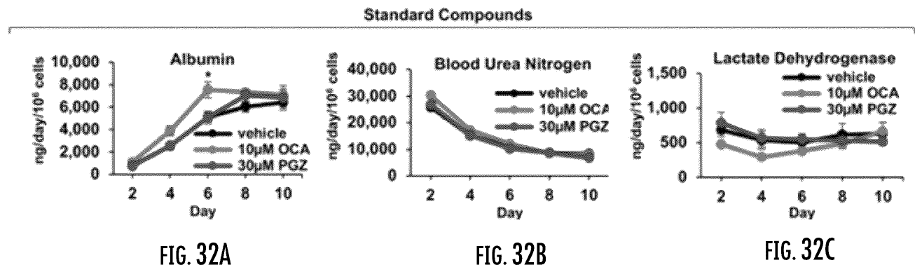

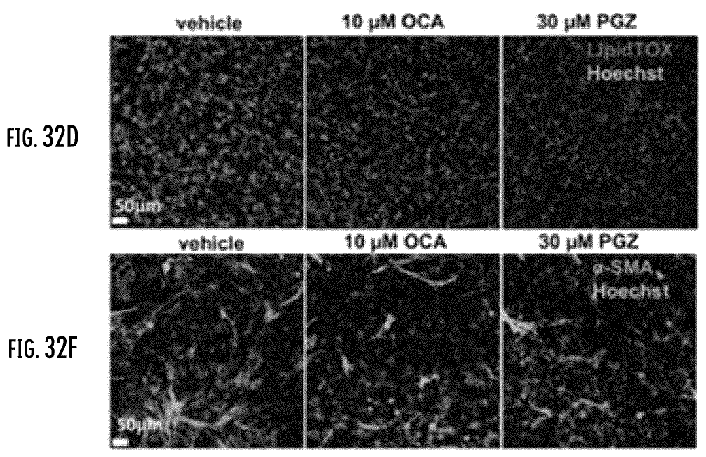

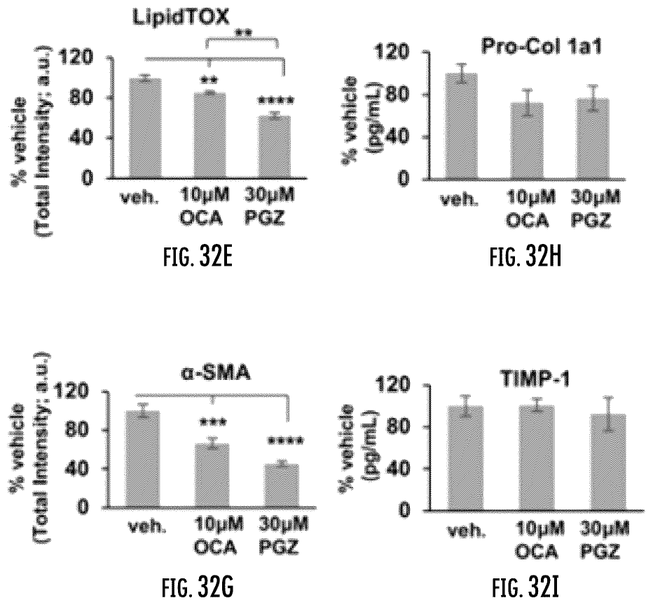

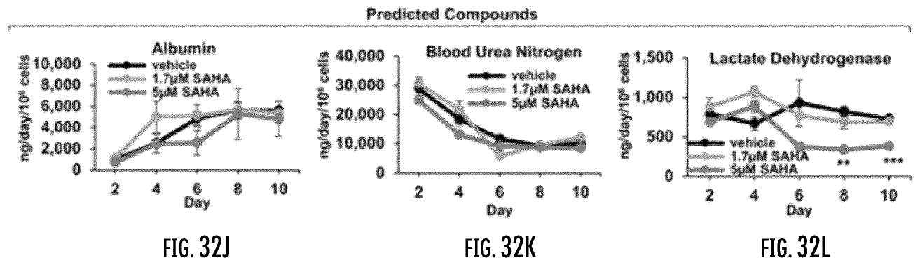

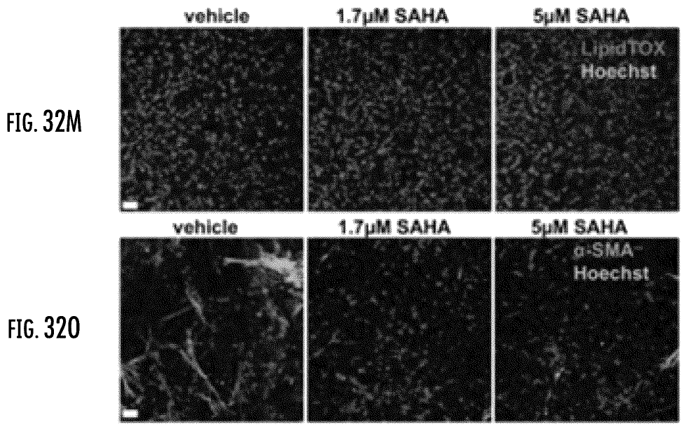

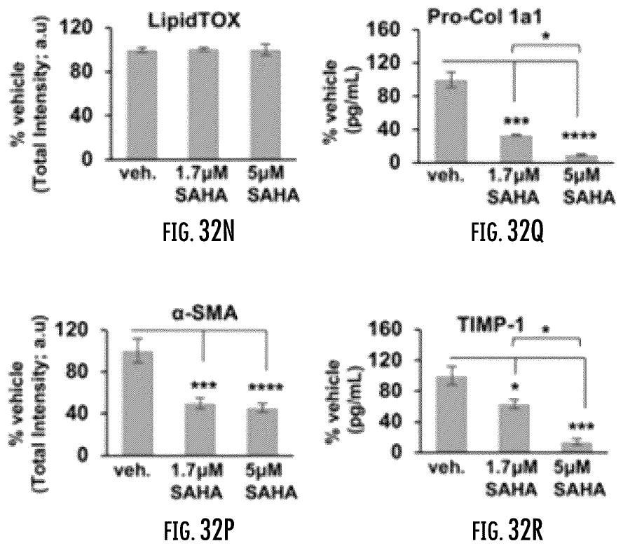

FIGS. 32A-32S illustrate control and predicted drugs reduce different NAFLD disease phenotypes in LAMPS models treated with EMS media. LAMPS models were maintained for 10 days in Early Metabolic Syndrome (EMS) media containing either vehicle control, 10 μM obeticholic acid (OCA) and 30 μM Pioglitazone (PGZ) [standard compounds], or vorinostat (suberoylanilide hydroxamic acid; SAHA) at 1.7 μM or 5 μM [predicted compounds]. A panel of metrics were examined to monitor disease-specific phenotypes. For standard drugs, albumin, blood urea nitrogen, and lactate dehydrogenase curves throughout the time course show similar profiles throughout the time course between vehicle and drug treatment groups, suggesting no overt model cytotoxicity or loss of function (FIGS. 32A-C). At the day 6 timepoint, there was a significant increase in albumin secretion in the OCA group; however, not further significant increases in albumin output were observed at later time points (days 8 and 10). At day 10, there is a significant decrease in steatosis (FIGS. 32D & E; LipidTOX™ intensity) and stellate cell activation (FIGS. 32F & G; α-SMA intensity) for both OCA and PGZ groups compared to vehicle. FIGS. 32D & F display representative 20× Day 10 LipidTOX™ (FIG. 32D) and α-SMA (FIG. 32F) images of LAMPS; Scale bar; 50 μm. There is no significant change in the secreted levels of the pro-fibrotic markers Pro-Col 1a1 (FIG. 32H) TIMP-1 (FIG. 32I) in either treatment group compared to vehicle. For the predicted drug vorinostat (SAHA), albumin and blood urea nitrogen curves show no significant differences between vehicle and treatment groups (FIGS. 32J & K), suggesting that these drug treatments do not result in loss of model functionality; however, a significant decrease in LDH secretion (FIG. 32L) at days 8 and 10 in the 5 μM vorinostat treatment group, suggesting decreased cytotoxicity. This is further supported by the significant decrease in stellate cell activation (FIGS. 32O & P; α-SMA intensity), production of the pro-fibrotic markers pro-collagen 1a1 and TIMP-1 (FIGS. 32Q & R) and inflammatory cytokine production (FIG. 32S) observed in the vorinostat group. In contrast, vorinostat does not reduce lipid accumulation compared to vehicle control (FIGS. 32M & N), indicating no effect on steatosis. FIGS. 32M & O display representative 20× Day 10 LipidTOX™ (FIG. 32D) and α-SMA (FIG. 32F) images of LAMPS under each treatment condition; Scale bar; 50 μm. For each control and drug treatment group, n=3 chips were analyzed and plotted+/−SEM for each assay and statistical significance was assessed using a One-Way ANOVA with Tukey's test (*p<0.05; ** p<0.01; *** p<0.001; **** p<0.0001).

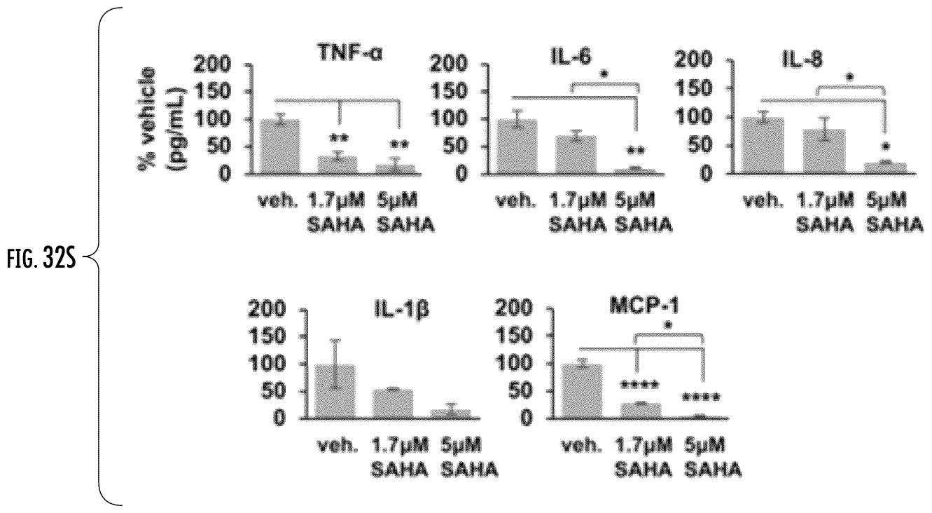

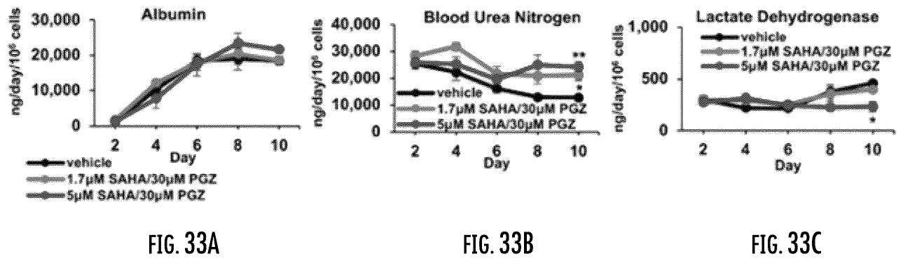

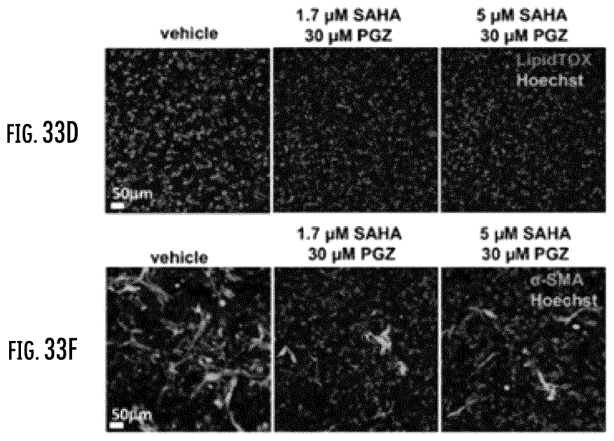

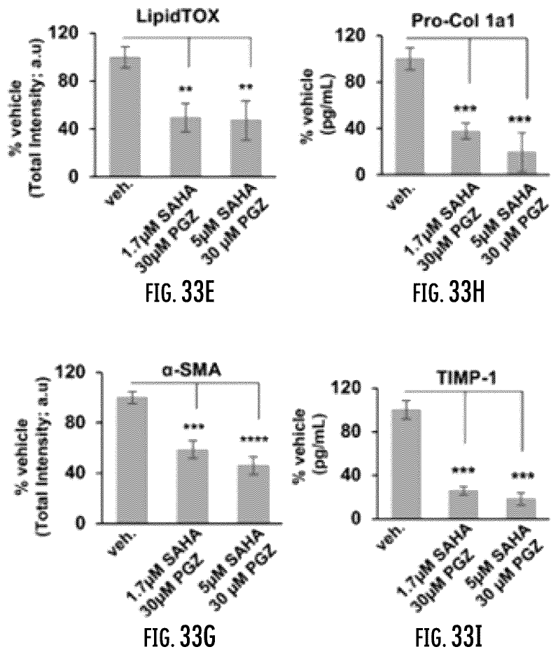

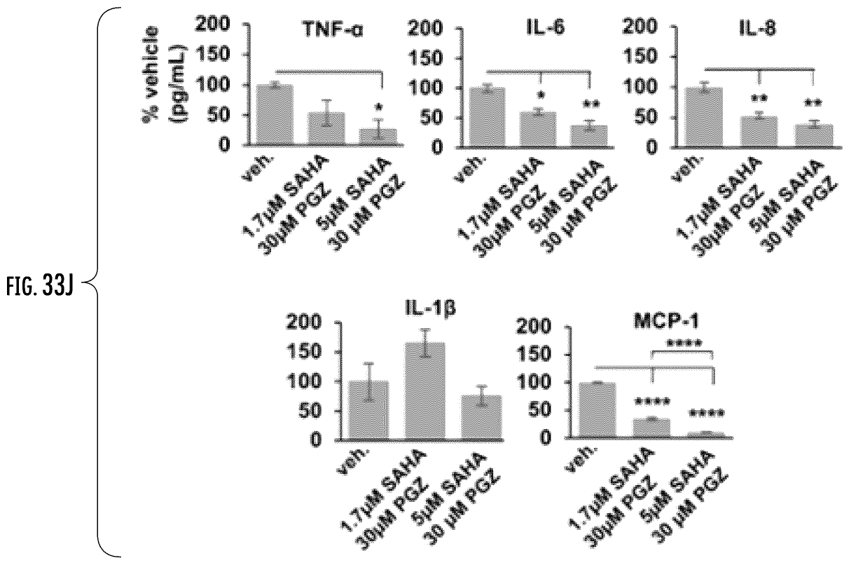

FIGS. 33A-33J illustrate pioglitazone and vorinostat used in combination results in the reduction of steatosis and stellate cell activation as well as the secretion of pro-fibrotic markers and production of inflammatory cytokines in LAMPS models treated with EMS media. LAMPS models were maintained for 10 days in NAFLD disease media containing combinations of pioglitazone (30 μM) and vorinostat (1.7 μM or 5 μM) or DMSO vehicle control. A panel of metrics were examined to monitor disease-specific phenotypes under these treatment conditions. While albumin secretion profiles show no significant differences between vehicle and drug treatment groups, suggesting that these drug combinations do not result in loss of model functionality (FIG. 33A), a significant increase in urea nitrogen secretion is observed in both drug combination groups compared to control, suggesting increased model metabolic activity (FIG. 33B). In addition, like the LDH profile in FIG. 32, there is a significant decrease in LDH secretion (FIG. 33C) in the 5 μM vorinostat treatment group, suggesting a reduction in cytotoxicity. Compared to the contrasting effects observed in the individual drug testing studies shown in FIG. 32, we observe an overall decrease in both lipid accumulation (FIGS. 33D & E) and stellate cell activation (FIGS. 33F & G), as well as in the production of the pro-fibrotic markers pro-collagen 1a1 and TIMP-1 (FIGS. 33H & I) and inflammatory cytokine production (FIG. 33J) when pioglitazone and vorinostat are used in combination. FIGS. 33D & F display representative 20× Day 10 LipidTOX™ (FIG. 33D) and α-SMA (FIG. 33F) images of LAMPS under each treatment condition; Scale bar; 50 μm. For each control and drug treatment group, n=3 chips were analyzed and plotted+/−SEM for each assay and statistical significance was assessed using a One-Way ANOVA with Tukey's test (* p<0.05; ** p<0.01; *** p<0.001; **** p<0.0001).

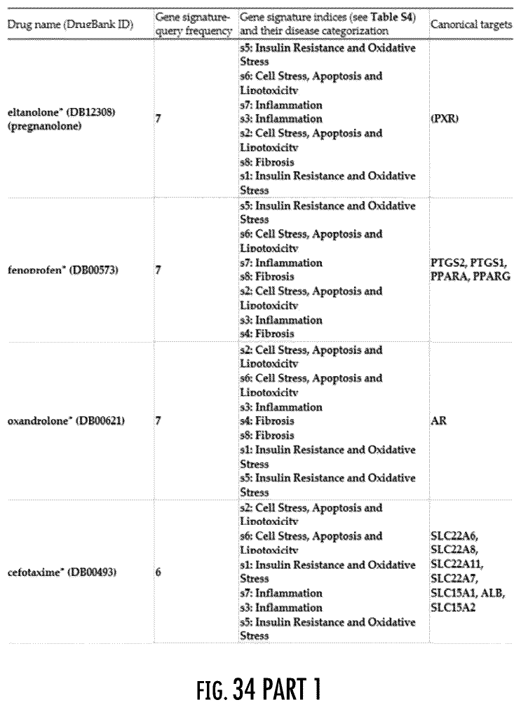

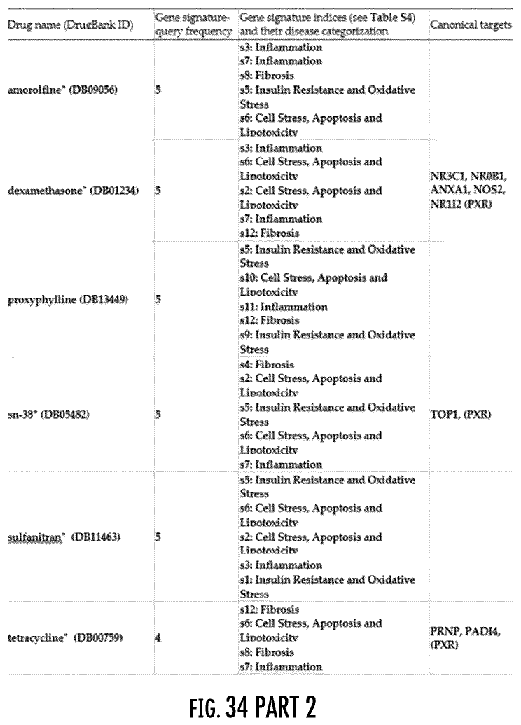

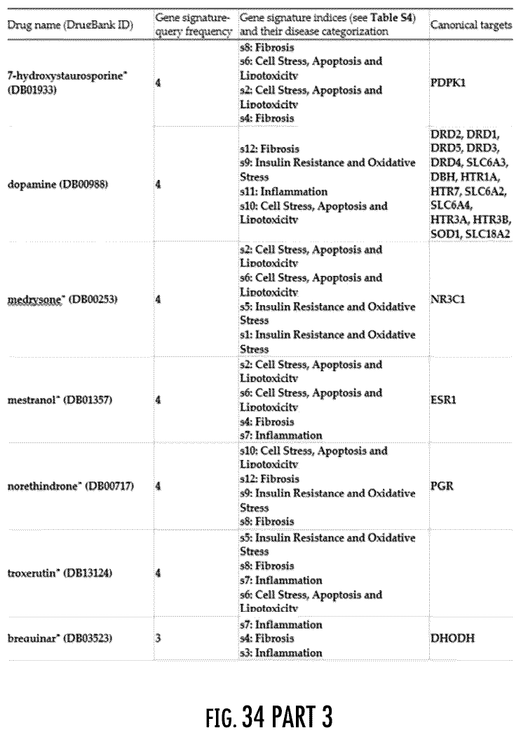

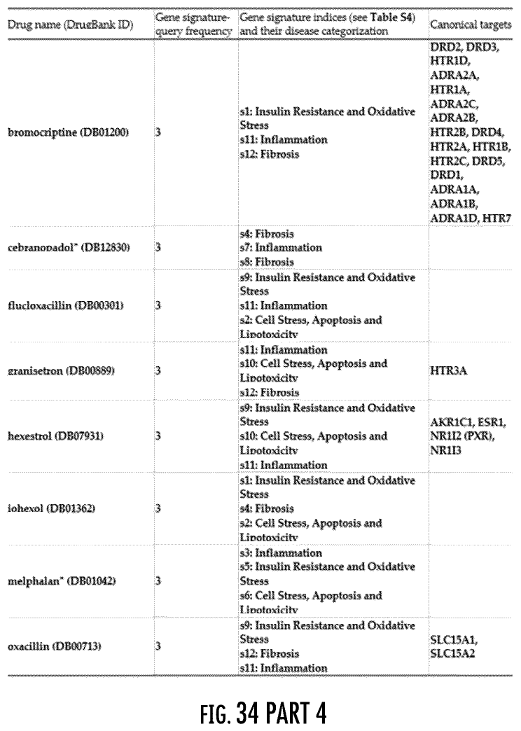

FIG. 34 is Table 6 illustrating the 25 highest ranked CMap-predicted drugs based on frequency of occurrence across multiple NAFLD-associated gene signature queries. Drugs/small molecules perturbagens identified in more than 1 of the 12 cluster-based gene signature queries were prioritized according to the number of occurrences across the 12 queries and termed: Gene signature-query frequency (Data File S4-S5). Each signature-based query is indexed s1-12 (see Table S4 and Data file S3) and ordered (from highest to lowest) according to the relative rank of the drug within each query that the drug was identified (i.e., occurrence). Each gene signature-based query is associated with a predominate feature (i.e., disease category) of NAFLD (see Table S4; Data File S3, and Methods). The canonical targets derive from DrugBank (v5.1.4) except for (PXR). Distinct from Table S5 CMap scores were calculated as percentile scores (see Methods, Results, and [Subramanian, A., et al. 2017]) and the 2020 expanded LINCS Database was used as indicated in the Methods and Results. * Denotes compounds also found in a parallel top 25 CMap-predicted drug analysis using clinical classification-based signature queries (Table S4 and Data File S3).

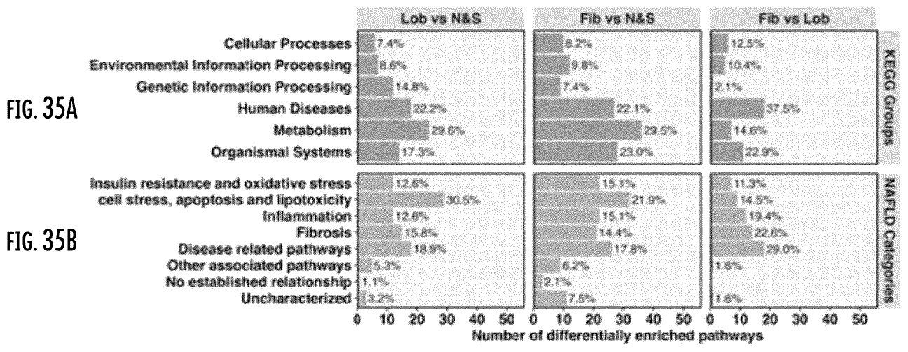

FIGS. 35A-35C illustrates the distribution of differentially enriched pathways and their respective KEGG groups and NAFLD categories of pairwise comparisons performed using the patient clinical classifications (complements FIGS. 5A-5C). The number of differentially enriched pathways identified between the Lobular inflammation vs Normal & Steatosis (Lob vs N&S), Fibrosis vs Normal & Steatosis (Fib vs N&S), and Fibrosis vs Lobular inflammation (Fib vs Lob), pairwise comparisons were 81, 122, and 48, respectively (adj. p-value<0.001). Their distribution (and percent contribution) with respect to KEGG Groups (FIG. 35A) and NAFLD categories (FIG. 35B) are detailed in Table S3 and Data file S2. The top ten differentially enriched pathways for each comparison (ranked by the FDR adjusted p-values through the linear modelling equivalent of a two sample, moderated t-test) are shown along with their association (black circles) with NAFLD categories C1-4 (as indicated and defined herein) (FIG. 35C). The colors of the bars represent the directionality and relative enrichment of each pathway for each of the pairwise comparisons.

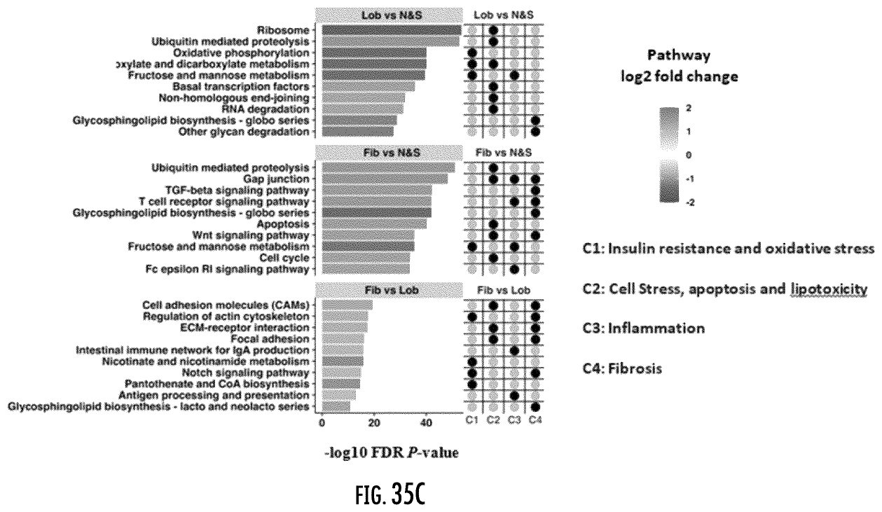

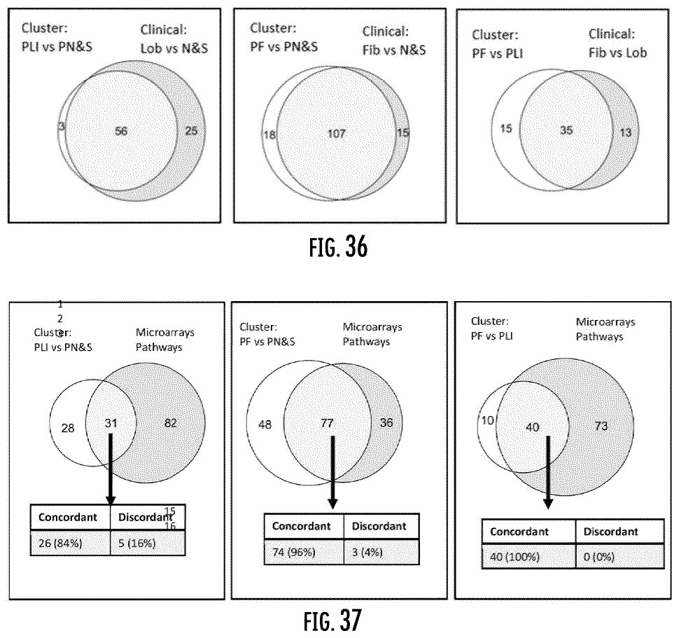

FIG. 36 are Venn diagrams showing the overlap of differentially enriched pathways (FDR p-value<0.001) identified in the cluster (left circle) and clinical label (right circle) pairwise comparisons (Supports FIGS. 5A-5C & FIGS. 35A-35C).

FIG. 37 shows a concordance analysis of the differentially enriched pathways in the cluster pairwise comparisons (left circle) and pathway list derived from microarray datasets (right circle). The microarray pathway list is the combined differentially enriched pathways found from re-analyzing the following datasets (the specific pairwise comparisons are indicated in the parenthesis): Ahrens et al., (2013) (NASH vs healthy obese), Arendt et al., (2015) (NASH vs simple steatosis), Murphy et al., (2013) (Advanced vs mild fibrosis). Differentially enriched (FDR p-value<0.05) pathways in the 182 patient cohort were considered concordant if they were also differentially enriched in the same direction (i.e., up-regulated or down-regulated) in one or more of the microarray cohorts. Conversely, discordance indicates that a pathway is still differentially enriched but in opposite directions. **p-value<=0.004 (Exact Binomial Test, % is estimated effect size).

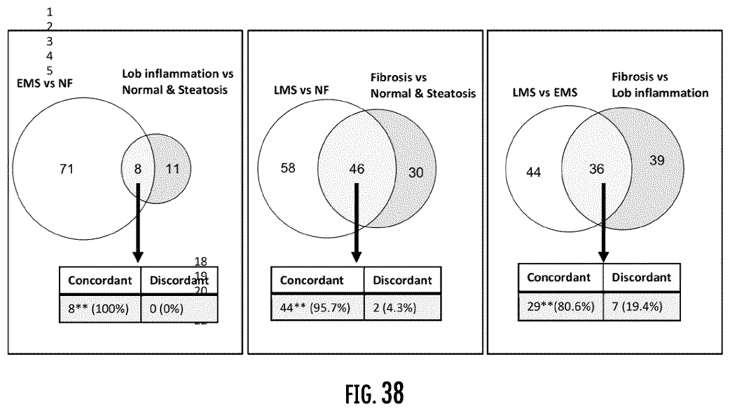

FIG. 38 shows a concordance analysis of the differentially enriched pathways in the LAMPS (left circle) and phenotypically matched patient pairwise comparisons. The pathways were identified using GSEA as described in the Methods for the pairwise comparisons. A pathway was considered concordant if it was significantly regulated (FDR p-value<0.05) in the same direction (up/down) in the LAMPS and patient comparisons, discordance is when pathways are differentially expressed but have opposite signs. **p-value<=0.004 (Exact Binomial Test, % is estimated effect size).

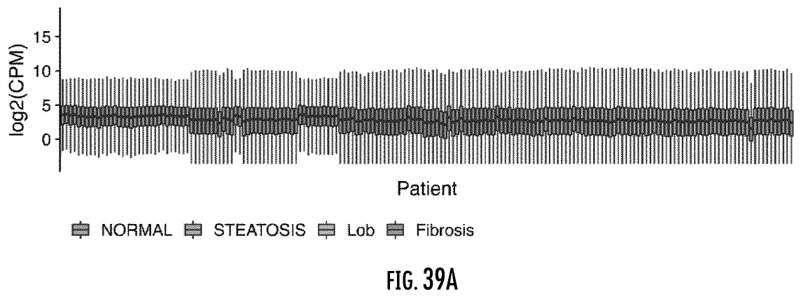

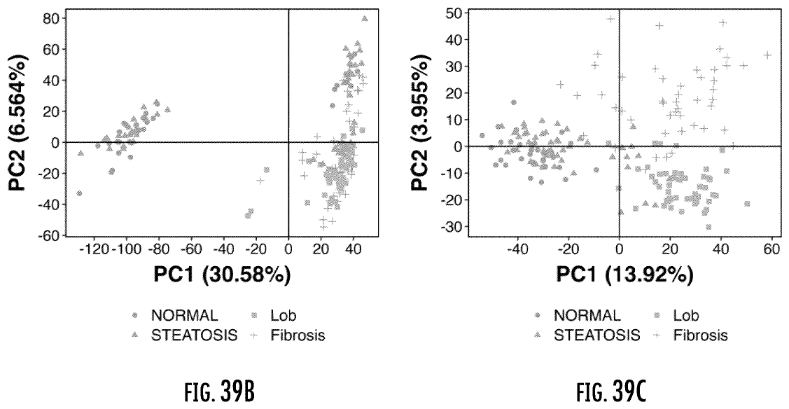

FIGS. 39A-39C illustrate an exploratory data analysis and PCA of the patient transcriptome. FIG. 39A shows the boxplots (outliers are not shown) of the log 2 transformed counts per million log 2 (CPM) gene expression values for each patient, ordered by the patient ID (i.e., the order the samples were processed). The distributions of normal and steatosis patients tend vary in discrete blocks of samples in contrast to lobular inflammation, fibrosis, or a set of steatosis patients collected later on in the experiment. This suggests the presence of a technical artifact which affects the distribution that is confounded with the patient classifications. Hence, we used quantile normalization to correct for this effect. Principal component analysis (PCA) of the log 2 (CPM) gene expression values revealed the presence of a batch effect (FIG. 39B). We therefore used surrogate variable analysis (SVA) to estimate covariates that could account for this unwanted heterogeneity while still retaining the biological variation. FIG. 39C shows the PCA plot using the SVA corrected gene expression matrix.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

To predict disease progression and response to emerging therapies for MAFLD, some members of the research community have adopted systems-based approaches, such as quantitative systems pharmacology (QSP). QSP can comprehensively and unbiasedly integrate molecular, cell, and clinical data to generate predictive models of disease progression (Mardinoglu, Boren et al. 2018). These computational models can then be iteratively tested in experimental models to identify emergent disease-specific networks and predictive biomarkers mechanistically linked to MAFLD pathogenesis (Wooden, Goossens et al. 2017). An overarching goal of implementing a QSP approach for addressing MAFLD heterogeneity is to identify MAFLD subtypes having distinguishable mechanisms of disease progression. It is hypothesized that a data-driven disease sub-classification that has remained elusive thus far (Friedman, Neuschwander-Tetri et al. 2018) will enable precision medicine and therapeutic advances based on targeting patient cohorts with distinct drug combinations (Stern, Schurdak et al. 2016, Taylor, Gough et al. 2019).

The integration of molecular, cell, and clinical data has begun to generate molecular signatures for MAFLD progression (Hoang, Oseini et al. 2019) but the experimental testing of predicted mechanistic hypotheses and therapeutic strategies has been limited by the availability of preclinical experimental models that recapitulate critical aspects of the human disease (Mann, Semple et al. 2016, Friedman, Neuschwander-Tetri et al. 2018). For example, whereas steatosis can be recapitulated in murine models, fibrosis, a key clinical biomarker of NASH progression, is not generally observed in these preclinical models (Sanyal 2019). Furthermore, even if significant fibrosis was observed in animal models (Asgharpour, Cazanave et al. 2016), it is unlikely that this pathogenesis would mimic the disease heterogeneity observed in the clinic.

To meet the need for developing preclinical patient-specific NAFLD models, human liver microphysiological systems (MPS) that recapitulate critical aspects of normal liver acinus multicellular architecture and function have been developed (Gough A 2021). When these systems are perfused with non-esterified fatty acids, glucose, insulin, and inflammatory cytokines mimicking a metabolic syndrome milieu that promotes hepatic insulin resistance, clinically relevant NASH-like changes have been observed (Feaver, Cole et al. 2016, Kostrzewski, Maraver et al. 2020). These changes include increases in de novo lipogenesis, gluconeogenesis, and oxidative and ER stress, and production of inflammatory and fibrogenic cytokines accompanied by hepatocyte injury and enhanced stellate cell activation. Overall, human biomimetic liver MPS containing multiple key liver acinus cells organized to recapitulate the liver acinus appear to mirror key aspects of MAFLD progression and provide a model consistent with the conceptual framework that MAFLD represents the hepatic expression of the metabolic syndrome in the majority of patients (Vernetti, Senutovitch et al. 2016, Lee-Montiel, George et al. 2017, Vernetti, Gough et al. 2017, Li, George et al. 2018, Gough A 2021, Mohammed 2021).

Described below is an implementation of a QSP-based platform (Stern, Schurdak et al. 2016, Taylor, Gough et al. 2019) to identify drugs that can be repurposed for NAFLD (FIG. 1). This iterative computational and experimental platform starts with the analysis of individual patient-derived hepatic RNAseq data encompassing a full spectrum of MAFLD disease states from simple steatosis, to NASH, advanced fibrosis and cirrhosis including associated comorbidities such as Type 2 diabetes (Gerhard, Legendre et al. 2018). This analysis has enabled the association of three distinct clusters of gene and pathway expression patterns with three MAFLD sub-classifications: normal & steatosis (N&S), predominantly lobular inflammation (PLI) and predominantly fibrosis (PF) (FIG. 3). Differential gene expression signatures specifically associated with states of MAFLD progression are derived. Then approved and investigational drugs that are predicted to normalize these gene signatures are identified through connectivity mapping (CMap) (Subramanian, Narayan et al. 2017, Keenan, Jenkins et al. 2018) and prioritized for experimental testing using two complementary approaches (FIG. 1). One approach is based on the frequency of appearances and rank that each predicted drug has across multiple signatures in conjunction with its potential for pleiotropic modulation of MAFLD-associated dyshomeostasis. The other prioritization approach maps known targets of these repurposable or candidate drugs to a MAFLD subnetwork independently constructed from genes differentially expressed during MALFD progression to then rank drugs according to network proximity (FIG. 1) (Guney, Menche et al. 2016). These approaches assist in prioritizing candidate drugs for testing in the human biomimetic liver MPS to 1) provide experimental proof-of-concept for the computational paradigm; 2) identify drugs and combinations that could form the basis for developing new MAFLD therapeutic strategies; and 3) gain mechanistic insights into the heterogeneity of MAFLD pathophysiology to enable precision medicine with novel therapeutics based on the repurposed drugs.

### Methods

### Generation of Individual Patient Liver Gene Expression Profiles

The RNA-seq data are derived from samples of wedge biopsies taken from the livers of patients undergoing bariatric surgery as previously described (Gerhard, Legendre et al. 2018). Patients were diagnosed, and samples were labeled, according to the predominant liver histology finding as normal, steatosis, lobular inflammation, or fibrosis (Gerhard, Legendre et al. 2018). The patient cohort is summarized in Table S2 and the data pre-processing steps are depicted in the context of the QSP workflow (FIG. 1, Box A). Paired fastq-files were pseudoaligned to the human Ensembl (Frankish, Vullo et al. 2017) v94 transcriptome using Kallisto (Bray, Pimentel et al. 2016) following the recommended pipeline. Estimated transcript abundances were then summarized into gene-level estimates using Tximport (Soneson, Love et al. 2015) with the settings recommended for VOOM (Law, Chen et al. 2014, Ritchie, Phipson et al. 2015). The data was then quantile normalized and surrogate variable analysis (SVA) (Leek and Storey 2007, Leek, Johnson et al. 2012) was used to identify batch effects.

Identification of Clusters of KEGG Pathway Expression Profiles Associated with NAFLD Subclasses

The pathophysiology of NAFLD is intrinsically complex and heterogeneous involving a complex interplay of diverse signaling pathways. The gene expression values for each patient sample were mapped to MSigDB v7.0 C2 KEGG pathways (Liberzon, Subramanian et al. 2011) using gene set variation analysis (GSVA) (Hanzelmann, Castelo et al. 2013) (FIG. 1, Box B). GSVA, being an intrinsically unsupervised method, enables individual patient pathway enrichment profiles to be generated across a heterogeneous population providing an advantage over GSEA, for example. Importantly and despite the known patient heterogeneity intrinsic to NAFLD, this classification was sufficient to identify and order the three clusters of distinct pathway enrichment profiles with different stages of NAFLD progression. The resulting sample vs pathway enrichment profile matrix was subjected to hierarchical clustering, and new groups were identified by cutting the column dendrogram at the 3rd level (FIG. 3). These clusters were then associated with the patient clinical data (Table S2) and named according to the predominant patient sub-classification in each cluster: one is almost entirely normal & steatosis (N&S) patients, the second is predominately lobular inflammation (PLI), and the third is predominately Fibrosis (PF) patients.

### Identification of Differential Gene Expression Signatures for the Three Pairwise Comparisons of NAFLD Subclasses

Differentially expressed genes (DEGs), were identified by initially row scaling the gene expression data and then applying the standard LIMMA-VOOM pipeline (FIG. 1, Box C) (Smyth 2004, Law, Chen et al. 2014, Ritchie, Phipson et al. 2015) for the three pairwise comparisons (PLI vs. N&S, PF vs. N&S, and PF vs. PLI) (Data file S1). Differentially enriched pathways were identified in the same way except that the GSVA outputs were used instead of gene expression data (FIG. 4; Table S2, and Data file S2). Each differentially enriched pathway was assigned to one or more of seven distinctly annotated categories based on literature mining of processes associated with NAFLD as follows: insulin resistance and oxidative stress (C1), cell stress, apoptosis and lipotoxicity (C2), inflammation (C3), fibrosis (C4), disease related pathways (C5), other associated pathways (C6), and no established relationship (C7) (FIG. 1, Box D). The first four categories (C1-C4) were used for the subsequent generation of gene signatures because they comprise our current conceptual framework of MAFLD progression (Sanyal 2019). These gene signatures were created by identifying differentially expressed genes (FDR p-value<0.001) which belonged to differentially enriched pathways (FDR p-value<0.001) in categories C₁-C₄ (FIG. 1, Box E; FIG. 12, and Data file S3). For each of the three pairwise comparisons (PLI vs. N&S, PF vs. N&S and PF vs. PLI), four pathway category-specific gene signatures were generated containing the aggregated up- and down-regulated genes (12 in total; FIG. 5; Table S3, and Data file S3). An analogous set of gene signatures was derived from the 3 pairwise clinical classification comparisons Lobular inflammation vs Normal & Steatosis (Lob vs. N&S), Fibrosis vs Normal & Steatosis (Fib vs. N&S), and Fibrosis vs Lobular inflammation (Fib vs. Lob). (FIG. 5; Table S3, and Data file S3). In sum, two sets of 12 differentially expressed gene signatures were generated, one set derived from distinguishable pathway enrichment profiles associated with different clinical subtypes and the other set derived directly from the clinical classifications. The differentially expressed genes in each signature reflect pathway dysregulation in NAFLD-associated processes and the signatures themselves are indicative of a particular disease state at different stages of disease development.

### Comparative Pathway Analysis Using Additional NAFLD Patient Datasets

We first performed an internal validation of our pathway results using the 3 pairwise cluster identified patient groupings (PLI vs. PN&S, PF vs. PN&S, and PF vs. PLI) by comparing them to pathway results using 3 pairwise clinical classification comparisons (Lob vs. N&S, Fib vs. N&S, and Fib vs. Lob) (FIG. 36). We found that 70-95% of pathways overlapped, and they were all concordant (enriched in the same direction in the cluster grouping and clinical pairwise comparison) (FIG. 36).

We further validated our pathway results (using the cluster groupings) (FIG. 1, Box C, FIG. 3; FIG. 6, and Data file S2) by performing concordance analysis on pathway results obtained from re-analyzing 3 external patient microarray datasets: (NASH vs healthy obese), (NASH vs simple steatosis), (Advanced vs mild fibrosis) (FIG. 37). This was done by identifying differentially expressed genes using the standard LIMMA protocol, then ranking genes by t-statistic and performing GSEA using the MSigDB v7.0 C2 KEGG pathways. In comparison to GSVA, GSEA is better suited to accommodate the smaller number of patient samples per clinical classification in the microarray datasets and accordingly identified more pathways with small effect sizes as being significant. We compared these differentially enriched (FDR p-value<0.05) pathway results to those in the 182 patient cohort (FIGS. 1, Box C, 3; FIG. 6, and Data file S2), in which a pathway was considered concordant if they were also differentially enriched in the same direction (i.e., up-regulated or down-regulated) in one or more of the microarray cohorts (FIG. 37). Conversely, discordance indicates that a pathway is still differentially enriched but in opposite directions (FIG. 37).

### Drug and Small Molecule Perturbagen Predictions Using CMap Analysis

The 12 gene signatures obtained in the previous step were used to query the LINCS L1000 level 5 (GSE92742) expression database (Subramanian, Narayan et al. 2017) as the connectivity mapping (CMap) resource to identify drugs and small molecule perturbagens that can potentially normalize the disease state by inverting these disease-associated signatures (FIG. 1, Box F; FIG. 18, and (Lamb, Crawford et al. 2006)). This database consists of perturbation instances, which is the gene expression output from a unique combination of cell type, time-point, compound, and compound concentration (Subramanian, Narayan et al. 2017). A separate database of LINCS compounds was created that could be mapped to DrugBank (v5.1.4) annotations. This was done by matching the compounds by compound common name, then by SMILES and/or Pubchem ID in cases where the common name differed between databases. In total, 1103 DrugBank compounds could be matched to 1495 LINCS compound IDs (there were cases of multiple different LINCS compound IDs for the same compound in the DrugBank database). Using this LINCS-DrugBank database, the L1000 database was filtered such that compounds with a DrugBank (v5.1.4) (Wishart, Feunang et al. 2018) annotation were retained; this yielded a set of 41,710 perturbation instances describing the response to 1103 compounds (as defined as a unique DrugBank ID), for 70 cell types, at 6 & 24 hr time-points, and a range of concentrations. Separately, AS-601245 (Carboni, Hiver et al. 2004) and its 64 associated perturbation instances were retained although this compound did not have a DrugBank entry. The similarity between each of these perturbation instances and the input gene signature was measured by two enrichment scores, one for up-regulated genes (ESᵤₚ) and the other for down-regulated genes (ES_{down}). ESᵤₚ and ES_{down} were combined into an overall CMap score (CS) as follows: If the sign of ESᵤₚ and ES_{doc}n are the same, CS=0; otherwise, CS=(ESᵤₚ−ES_{down})/2 (Subramanian, Narayan et al. 2017). This yielded a list with a 41,774 CSs (one for each perturbation instance), for each of the 12 gene signatures (see above and FIG. 18). The results were further filtered to remove instance predictions with a CS of 0, and compiled into a csv file (Data File S4). Since the overall goal is to identify drugs that normalize the disease state by inverting the disease gene expression signature, drugs and small molecule perturbagens corresponding to the most negative CSs were prioritized (Tables S5) (Lamb, Crawford et al. 2006). P-values for the CSs were calculated using methods adapted from Chen et al., 2018 (Chen, Wei et al. 2017). For each gene signature, a distribution of random CSs was generated by calculating the CS between a random perturbation instance and random gene set with the same number of up- and down-regulated genes as the gene signature. This was performed up to 500,000 times for each gene signature, and were used to calculate a p-values for each CS. The p-values represent the probability of observing a CS at least as extreme using a random set of genes with the same size as the gene signature. The p-values were then adjusted for multiple testing using the FDR method (Benjamini and Hochberg 1995).

An initial drug prioritization of the resulting CMap predictions (Data File S4) was performed by retaining the top 20 drugs predicted from each signature with an FDR p-value<0.05, (Table S5; Data file S5). This resulted in a list of 196 predictions (Data file S5), containing 139 unique compounds, as some compounds appeared in more than 1 signature. The 139 compounds were then ranked based on their frequency of appearance across the 12 signatures (Table 1).

During the course of our initial studies, an updated and expanded 2020 LINCS database was released (see clue.io) that we used to generate a 2020 LINCS-DrugBank database. This version included the 1103 previously matched compounds and an additional 1033 compounds yielding 334,393 instances comprising 2136 DrugBank compounds (2795 LINCS compounds IDs) across 228 cell types, and a range of time-points and concentrations (clue.io). We performed a similar approach as described above in a follow up study using the expanded 2020 LINCS database (accessible at clue.io), except compounds were ranked (in ascending order) using the maximum quantile summary score (FIG. 1, Box G, Table 6 and Data file S5). The top 25 predictions from the follow up study are shown Table 6.

### Drug and Small Molecule Prioritization Using Network Proximity Analysis

Network proximity is used to evaluate the potential pharmacological significance of the network distance between a drug's target profile and a given disease module (Guney, Menche et al. 2016). The methodology (Guney, Menche et al. 2016) is based on the premise that a drug is effective against a disease by targeting proteins within or in the immediate vicinity of the corresponding disease module. In essence, this approach provides an independent criterion for increasing the specificity of the CMap analysis to enable further drug prioritization for experimental testing (FIG. 1 Unit 3). For determining network proximity, a representation of the liver specific protein-protein interaction (PPI) network (referred to as the background network) is required. The liver BioSnap network (Marinka Zitnik and Leskovec 2018) which contains 3,180 nodes and 48,409 edges was used. A subnetwork from this background network representing the PPIs specific to NAFLD was then generated as follows: we selected the KEGG pathway map of NAFLD which represents a stage dependent progression of NAFLD (pathway id: hsa04932, (Kanehisa and Goto 2000, Kanehisa, Furumichi et al. 2017)) in addition to 10 interrelated pathways that included: TNF-signaling (hsa04668, (Kanehisa and Goto 2000, Kanehisa, Furumichi et al. 2017)), insulin signaling (hsa04910, (Kanehisa and Goto 2000, Kanehisa, Furumichi et al. 2017)), Type II diabetes mellitus (hsa04930, (Kanehisa and Goto 2000, Kanehisa, Furumichi et al. 2017)), PI3K-Akt signaling (hsa04151, (Kanehisa and Goto 2000, Kanehisa, Furumichi et al. 2017)), adipocytokine signaling (hsa04920, (Kanehisa and Goto 2000, Kanehisa, Furumichi et al. 2017)), PPAR signaling (hsa03320, (Kanehisa and Goto 2000, Kanehisa, Furumichi et al. 2017)), fatty acid biosynthesis (hsa00061, (Kanehisa and Goto 2000, Kanehisa, Furumichi et al. 2017)), protein processing in the endoplasmic reticulum (hsa04141, (Kanehisa and Goto 2000, Kanehisa, Furumichi et al. 2017)), oxidative phosphorylation (hsa00190, (Kanehisa and Goto 2000, Kanehisa, Furumichi et al. 2017)) and apoptosis pathways (hsa04210, (Kanehisa and Goto 2000, Kanehisa, Furumichi et al. 2017)). We then created an initial subnetwork by taking the intersection between the background network and the genes from these 11 pathways, yielding 390 nodes. We further filtered this initial subnetwork to only include nodes that were differentially expressed in the 3 comparisons (PLI vs. N&S, PF vs. N&S and PF vs. PLI), resulting in a subnetwork of 234 nodes and 1,130 edges (see FIG. 19, Table S6, and Data File S6).

This NAFLD subnetwork was defined as the disease module and was used to determine the network proximity of the 139 CMap prioritized compounds (Table 1; Data File S5) described above. Among these, 91 are known to have target profiles that include liver-expressed proteins and constituted the set of drugs that underwent network proximity analysis (FIG. 1, Box J, Data file S7) as previously described (Guney, Menche et al. 2016) and summarized here. AS-601245 was also included in the network proximity with the targets MAP3K9, MAPK8/JNK1, MAPK9/JNK2, and MAPK10/JNK3. For the set of 234 NAFLD-associated subnetwork nodes (S) and each drug's set of targets (T) (as determined from DrugBank v5.1.4 (Wishart, Feunang et al. 2018)), the closest distance measured by the average shortest distance path between nodes s and the nearest node t in the human liver PPI interactome was calculated as:

$$d ( S , T ) = \frac{1}{‖ T ‖} {\sum }_{t ∈ T} {min}_{s ∈ S} d ( s , t )$$

A reference distance distribution was constructed, corresponding to the expected distance between two randomly selected groups of proteins of the same size and degree distribution as the original disease proteins and drug targets in the network. This procedure was repeated 1,000 times and the mean and standard deviation of the reference distance distribution were used to calculate a z-score by converting an observed distance to a normalized distance. Each drug was then assigned a z-score to rank its potential effects on NAFLD disease module, where a lower z-score represents a drug's target profile that is closer to the disease module. The output results are in Table S7 and Data file S8.

### Example 2

### Human Liver Acinus Microphysiology System (LAMPS) Studies

### Reagents

Matrix proteins: The extracellular matrix protein fibronectin was obtained from Millipore Sigma (Burlington, MA), rat-tail collagen type 1 from Corning (Franklin Lakes, NJ) and porcine liver extracellular matrix (LECM) was provided as a 10 mg/mL stock by the laboratory of Dr. Stephen F. Badylak, McGowan Institute for Regenerative Medicine, University of Pittsburgh (Pittsburgh, PA).

Media components: D-glucose solution, transferrin, selenium, glucagon, fetal bovine calf serum and bovine serum albumin were purchased from Sigma Millipore. Linoleic, oleic, and palmitic acids were also purchased from Sigma Millipore. William's E medium (no phenol red; 11.5 mM glucose), human recombinant insulin, penicillin streptomycin, HEPES and Gluta-MAX were purchased from ThermoFisher. In addition, a custom manufactured lot of D-glucose-, L-glutamine- and phenol red-free William's E medium was purchased from ThermoFisher. Lipopolysaccharide (LPS) was obtained from Sigma Millipore; TGF-β was purchased from ThermoFisher (Waltham, MA).

Fluorescent probes and antibodies: HCS LipidTOX Deep Red Neutral Lipid Stain (#H34477) and Hoechst 33342 (#H3570) were also obtained from ThermoFisher. Mouse monoclonal anti-smooth muscle actin (α-SMA; #A2457) was purchased from Sigma Millipore. AlexaFluor goat-anti mouse 488-conjugated secondary antibodies (#A32723) were purchased from ThermoFisher. All MPS staining procedures were carried out as previously described (Vernetti, Senutovitch et al. 2016, Lee-Montiel, George et al. 2017, Mohammed 2021).

Drugs: All compounds were purchased from Selleck Chemicals (Houston, TX): everolimus (#S1120); troglitazone (#S8432); GW9662 (#S2915).

LAMPS cell sources and cell culture. A single lot of selected cryopreserved primary human hepatocytes (lot #Hu1960) with >90% viability and re-plating efficiency post-thaw were purchased from ThermoFisher. A single lot of selected cryopreserved primary human liver sinusoidal endothelial cells (LSEC; lot #: HL160019) were purchased from LifeNet Health (formerly Samsara Sciences; Virginia Beach, VA.). The human monoblast cell line, THP-1, used to generate Kupffer cells, was purchased from ATCC (Rockville, MD). LX-2 human stellate cells were acquired from Sigma Millipore (Billerica, MA). The LX-2 cell is an immortalized human hepatic stellate cell that constitutively expresses key receptors regulating hepatic fibrosis, and proliferates in response to PDGF, a prominent mitogen contributing to liver fibrosis (Bonner 2004, Xu, Hui et al. 2005).

Primary human hepatocytes were thawed following the manufacturer's recommendations using Cryopreserved Hepatocyte Recovery Medium (CHRM; ThermoFisher) and were initially cultured in hepatocyte plating media (HPM) containing Williams E medium (11.5 mM glucose) supplemented with 5% fetal bovine serum, 100 mg/mL penicillin-streptomycin and 2 mM L-glutamine. For perfusion in LAMPS models, cells were maintained in normal fasting (NF), early metabolic syndrome (EMS), or late metabolic syndrome (LMS) media. The description of components of these media types is detailed in the Metabolic Syndrome Media section below. LSECs were thawed and expanded in endothelial cell basal medium-2 (EBM-2) supplemented with the endothelial growth medium-2 (EGM-2) supplement pack (Lonza; Alpharetta, GA; #CC-4176). THP-1 cells were cultured in suspension in RPMI-1640 medium (ThermoFisher) supplemented with 10% fetal bovine serum (FBS; ThermoFisher), 100 μg/mL penicillin streptomycin (ThermoFisher), and 2 mM L-glutamine (ThermoFisher). THP-1 cells were differentiated into mature macrophages by treatment with 200 ng/mL phorbol myristate acetate (Sigma Aldrich) for 48 h. Differentiated THP-1 monocytes release human tumor necrosis factor alpha (TNF-α) and interleukin-6 (IL-6) in response to LPS treatment, a condition reported to induce the immune mediated liver toxic response in in vitro models (Jang, Choi et al. 2006, Kostadinova, Boess et al. 2013). LX-2 cells were cultured in Dulbecco's Modified Eagle Medium (DMEM; ThermoFisher) supplemented with 2% FBS and 100 μg/mL penicillin streptomycin.

Metabolic syndrome media. We developed medias that were designed to create disease progression from Normal Fasting (NF) to early metabolic syndrome (EMS or NAFLD) and late metabolic syndrome (LMS or T2D) over a two-week period in the LAMPS platform (Mohammed 2021). For the studies described here, we used both NF and EMS media formulations. We developed the media around Williams E media that did not have glucose, insulin, glucagon, oleic acid, palmitic acid or molecular drivers of disease including TGF-β and LPS. We then adjusted these components to reflect the pathophysiological conditions as described below (Alford, Bloodm et al. 1977, Kuhl 1977, Kim, Kim et al. 2000, Chitturi, Abeygunasekera et al. 2002, de Almeida, Cortez-Pinto et al. 2002, Valencia, Marin et al. 2002, Edgerton, Ramnanan et al. 2009, Winnick, An et al. 2009).

Normal Fasting (NF) Media: NF media which was prepared in a custom formulation of William's E medium without glucose (ThermoFisher) supplemented with 5.5 mM glucose (Sigma Millipore), 1% FBS (Corning), 0.125 g/mL bovine serum albumin (Sigma), 0.625 mg/mL human transferrin, 0.625 μg/mL selenous acid, 0.535 mg/mL linoleic acid (Sigma), 100 nM dexamethasone (ThermoFisher), 2 mM glutamax, 15 mM HEPES (ThermoFisher), 100 U/100 μg/mL pen/strep (Hyclone Labs), 10 μM insulin (ThermoFisher) and 100 μM glucagon (Sigma).

Early Metabolic Syndrome (EMS) Media: Early metabolic syndrome (EMS) medium was derived from the NF media formulation with the following modifications: 11.5 mM glucose, 10 nM insulin, 30 μM glucagon, 200 μM sodium oleate and 100 μM palmitic acid (Sigma).

Late metabolic Syndrome (LMS) Media: Late metabolic syndrome (LMS) medium was derived from the NF media formulation with the following modifications: 20 mM glucose, 10 nM insulin, 30 μM glucagon, 200 μM sodium oleate and 100 μM palmitic acid (Sigma), 10 ng/ml TGF-β and 1 μg/ml lipopolysaccharide.

Preparation of fatty acids (FAs). FA coupling was performed as previously described (Busch, Cordery et al. 2002). Briefly, 18.4% BSA was dissolved in William's E media without glucose by gentle agitation at room temperature for 3 h. Palmitate (Sigma) or oleate (Sigma) (8 mmol/I) was then added as sodium salts, and the mixture was agitated overnight at 37° C. The pH was then adjusted to 7.4, and then, after sterile filtering, FA concentrations were verified using a commercial fatty acid quantification kit (BioVision, Milpitas, CA), and aliquots were stored at −20° C. until use.

LAMPS model assembly and maintenance. LAMPS studies were carried out as previously described (Vernetti, Senutovitch et al. 2016, Lee-Montiel, George et al. 2017, Miedel, Gavlock et al. 2019, Mohammed 2021) with modification to include the use of human primary liver sinusoidal endothelial cells (LSECs). A single chamber commercial microfluidic device (HAR-V single channel device, SCC-001, Nortis, Inc. Seattle, WA) was used for LAMPS studies. For all steps involving injection of media and/or cell suspensions into LAMPS devices, 100-150 μl per device was used to ensure complete filling of fluidic pathways, chamber and bubble traps. The percentages of hepatocytes, THP-1, LSEC, and LX-2 cells are consistent with the scaling used in our previously published models (Vernetti, Senutovitch et al. 2016, Lee-Montiel, George et al. 2017, Miedel, Gavlock et al. 2019, Mohammed 2021). For the studies described here, LAMPS models were set up and maintained for 10 days under flow. The experimental setup workflow is described in the Supplement.

Steatosis measurements. Steatosis measurements were performed using HCS LipidTOX Deep Red Neutral Lipid Stain (ThermoFisher) after completion of the experimental time course (Day 10) in LAMPS models as previously described (Lee-Montiel, George et al. 2017) and are outlined in detail in the Supplemental Methods section.

Stellate cell activation. Staining for LX-2 cell expression of α-smooth muscle actin (α-SMA) was performed after completion of the experimental time course (Day 10) in LAMPS models as previously described (Vernetti, Senutovitch et al. 2016, Li, George et al. 2018) and are outlined in detail in the Supplemental Methods section.

Secretome measurements. Efflux media from LAMPS devices was collected on days 2,4,6,8, and 10 to measure albumin, blood urea nitrogen, and lactate dehydrogenase. The enzyme linked immunosorbent assay (ELISA) for albumin was purchased from Bethyl Laboratories (Montgomery TX). The CytoTox 96 for lactate dehydrogenase (LDH) and the urea nitrogen test were purchased from Promega (Madison, WI) and Stanbio Laboratory (Boerne, TX), respectively. TNF-α (R&D Systems, Minneapolis, MN), Collagen 1A1 (R&D Systems, Minneapolis, MN) and TIMP-1 (R&D Systems, Minneapolis, MN) ELISA measurements were also made on efflux collected on days 2,4,6,8, and 10. All efflux measurements were carried out according to the manufacturer's protocol and obtained as described previously (Vernetti, Senutovitch et al. 2016, Lee-Montiel, George et al. 2017, Li, George et al. 2018, Miedel, Gavlock et al. 2019).

Statistical Analysis. Statistical comparisons between specific drug treatment groups were made by using PRISM (San Diego, CA) to perform One-Way ANAOVA analysis with Tukey's test to compare means of individual groups with a significance level a of 0.05 unless stated otherwise.

### Concordance Analysis of Differentially Enriched Pathways in Patients and LAMPS

The raw LAMPS transcriptome data were processed using the same pipeline as described for the patients (FIG. 1, Box L). Differentially expressed genes were identified using the standard LIMMA-VOOM protocol [Ritchie, M. E., et al. 2015, Law, C. W., et al. 2014] in which the genes were fit with a linear model for media treatment and timepoint. As we are interested in the treatment effects, time point was treated here as confounding variable [Law, C. W., et al. 2020]. We identified differentially expressed genes for LAMPS by performing three pairwise comparisons consisting of EMS vs NF, LMS vs NF, and LMS vs EMS, which are meant to be analogous to the patient pairwise comparisons (Lob vs. N&S, Fib vs. N&S, and Fib vs. Lob). The phenotypes of NF, EMS, and LMS range from minimal, moderate, and pronounced levels of steatosis, inflammation, and fibrosis, respectively [Saydmohammed, M., et al. 2021] (FIG. 31B). Differentially enriched pathways were identified by ranking the genes by t-statistic for each pairwise comparison and then performing GSEA [Subramanian, A., et al. 2005] using the MSigDB v7.0 C2 KEGG pathways [Liberzon, A., et al. 2011] for both the LAMPS and patient comparisons (FIG. 1, Box L, FIG. 38).

Using this differential enrichment pathway analysis as input, we performed a concordance analysis of the LAMPS and matched patient pairwise comparisons (FIGS. 1, Box L, FIG. 38). A pathway was considered concordant if it was significantly (FDR p-value<0.05) regulated in the same direction in both the LAMPS and matched patient pairwise comparisons (FIG. 38). Conversely, discordance indicates that a differentially enriched pathway identified in both comparisons, is regulated in opposite directions.

Comparing LAMPS NAFLD Model Transcriptomes to Patients Via Multinomial Logistic Regression with Elastic Net Penalization (MLENet)

We used an MLENet model [Friedman, J., T. Hastie, and R. Tibshirani 2010] to compare the LAMPS to patients since this is a classifier that performs feature (i.e., gene) selection (FIG. 1, Box L). The patient gene expression data was prepared by first ranking the genes by variance and taking the top 7,500 (this is done to reduce overfitting by removing uninformative features). The same variance thresholding was applied to the LAMPS expression matrix. Next genes which were not in both the variance filtered LAMPS and patient expression matrixes were removed from both, yielding a set of 4057 genes. For the LAMPS gene expression matrix, we used surrogate variable analysis [Leek, J. T. and J. D. Storey 2007, Leek, J. T. and et al. 2012] to predict and then remove unwanted sources of variation (timepoint, and possible cell ratio differences). Both the patient and LAMPS matrixes were standardized (gene-wise) to have zero mean and unit variance.

We used a nested cross-validation approach to ensure that MLENet could successfully differentiate between the 4 patient histological classifications (Normal, Steatosis, Lobular inflammation, or Fibrosis). To do this, we used the Glmnet package [Friedman, J., T. Hastie, and R. Tibshirani 2010] applying the appropriate distribution (multinomial) and setting (alpha=0.95) that in initial trials enabled optimal performance for classifying the LAMPS samples. The nested cross-validation was performed by first generating 100 sets of training and test data (FIG. 31A). This was done by sampling 70% of the patient from each class to create a training subset and then using the remaining 30% for the testing subset (FIG. 31A). For each of the 100 sets, we trained an MLENet model on the training subset using cv.glmnet [Friedman, J., T. Hastie, and R. Tibshirani 2010] and then used the testing subset to evaluate the model's performance by calculating the specificity and sensitivity of the 4 patient classes (FIG. 31A).

After ensuring that that the MLENet approach could accurately classify patients with a mean (numbers in parenthesis are standard deviation) specificity of 0.93 (0.03), 0.83 (0.03), 0.98 (0.02), 0.95 (0.03) for Normal, Steatosis, Lobular inflammation, and Fibrosis respectively, we trained a final model using the 182 patients using the parameters described above (FIG. 1, Box L, FIG. 31B). The final MLENet model selected 71 genes, of which, the majority (80%) had prior association with NAFLD in independent studies (usually being differentially expressed in other studies). We used this final MLENet model to classify the LAMPS samples as belonging to one of the 4 patient classes (FIG. 31B).

### Example 3

### Individual Patient KEGG Pathway Enrichment Profiles Cluster According to Predominant MAFLD Subtypes

The results of unsupervised clustering of KEGG pathway enrichment profiles from 182 patient samples representing different stages of MAFLD (NAFLD when the patient samples were studied-(Gerhard, Legendre et al. 2018) including 36 normal, 46 steatosis, 50 lobular inflammation and 50 fibrosis are shown (FIG. 3; Table S2). The dendrogram was cut at the third level, and this resulted in three clusters that were each significantly enriched in one of these stages (FIG. 3). Details of the sample composition of each cluster are shown in Table S2. The first cluster is composed of 43.3% normal patients and 48.1% patients with simple steatosis (NAFL), termed Normal & Steatosis (N&S), highlighting the challenge of distinguishing these two cohorts by gene expression analysis alone when inflammation is not discernable (FIG. 3; Table S2). The second cluster is predominated by patients with lobular inflammation (70.3%) with little or no fibrosis, termed Predominately Lobular Inflammation (PLI) (FIG. 3; Table S2). The third is comprised of patients with advanced disease having fibrosis, termed Predominately Fibrosis (PF) (FIG. 3; Table S2). The sample clustering is significantly associated (Pearson's Chi-squared Test) with MAFLD subclass (p<2.2e-16) and type 2 diabetes (T2D) status (p=0.01). FIG. 3 also shows that the distribution of sex, body mass index (BMI) and age are similar across the different clusters. In contrast, the occurrence of T2D in cluster PF (55%) is higher than in cluster N&S (32%) and PLI (32%), corroborating independent analyses of this particular cohort (Gerhard, Legendre et al. 2018) and other cohorts (Bazick, Donithan et al. 2015, Portillo-Sanchez, Bril et al. 2015, Kwok, Choi et al. 2016) that among individuals with type 2 diabetes and MAFLD, the prevalence of NASH and advanced fibrosis is enriched when compared to nondiabetics with MAFLD. This is most evident among the individual patients diagnosed with fibrosis in the PF cluster with 77% having T2D (Table S1).

We next investigated in more detail the association between distinct pathway enrichment profiles (i.e., molecular disease phenotypes) and clinical subtypes by determining the differential pathway enrichment profiles of the pairwise comparisons among the 3 clusters and among the corresponding clinical subtypes (FIG. 1, Box C).

The pairwise cluster comparisons of PLI vs. N&S, PF vs. N&S and PF vs. PLI gene and pathway expression data yielded a total of 139 unique differentially enriched pathways (FDR p-value<0.001) (FIG. 1, Box C; Data file S2). Analogously, the pairwise clinical subtype comparisons of Lobular inflammation vs Normal & Steatosis (Lob vs. N&S), Fibrosis vs Normal & Steatosis (Fib vs. N&S), and Fibrosis vs Lobular inflammation (Fib vs. Lob) gene and pathway expression data yielded a total of 140 unique differentially enriched pathways (FDR p-value<0.001) (Data file S2). The distributions of these differentially enriched pathways within their respective top-level KEGG hierarchical classifications in each pairwise comparison are presented in FIG. 5A and FIG. 35A, respectively. Overall, these distributions are consistent with the intrinsic heterogeneity of NAFLD that reflects the diverse but convergent impacts of the environment, metabolism, comorbidities, and genetic risk factors. More specifically, many of these differentially enriched pathways can be associated with at least one of four categories that comprise our current conceptual framework of NAFLD progression (FIG. 1, Box D, Methods): C1) Insulin resistance and oxidative stress, C2) Cell stress, apoptosis, and lipotoxicity, C3) Inflammation, and C4) Fibrosis (FIGS. 3B, S1B) [Friedman, S. L., et al. 2018, Sanyal, A. J. 2019]. Apart from these four main categories, other pathways have been observed that are less directly associated with NAFLD or the metabolic syndrome (FIG. 1 Box D, FIG. 5B, FIG. 35B).

The 10 most differentially enriched pathways for all patient subgroup pairwise comparisons, and their association with the disease processes within these four categories (C₁-C₄) are shown in FIG. 5C and FIG. 35C. The 10 pathways for the PF vs. N&S and the PLI vs. N&S cluster-based comparisons, and the Fib vs. N&S and the Lob vs. N&S clinical subtype comparisons, are consistent with the metabolic underpinning, and the resultant cellular stress and inflammatory response intrinsic to NAFLD pathogenesis. Complementarily, the differentially enriched pathways within the comparisons between PF vs PLI and between Fib vs. Lob are consistent with fibrosis being the widely recognized hallmark of disease progression in NASH (FIGS. 5C, 35C). The majority of the top 10 differentially enriched pathways in these comparisons have been shown to have a role in hepatic fibrosis [30-37] with several involved in hepatic stellate cell activation [30-32]. The majority of differentially enriched pathways derived from the unsupervised clusters are concordant with those derived from the clinical subtypes per se (FIG. 36), corroborating an association of these pathways with NAFLD progression. A meta-analysis extending the unsupervised cluster comparisons to three independent NAFLD patient cohorts further supports an association of many of these differentially enriched pathways with NAFLD progression (FIG. 37). The fraction of the top 10 differentially enriched pathways playing a role in multiple disease categories in the PF vs PLI comparison was greater than the fractions in the other two comparisons, indicative of enhanced disease complexity during progression (FIG. 5C). Together, the analysis of this transcriptomic data set appears to have corroborated the clinical relevance of these differentially enriched pathways in the context of the current conceptual framework of NAFLD progression [Friedman, S. L., et al. 2018, Sanyal, A. J. 2019].

Although each of these identified differentially enriched pathways has the potential to be a drug target, their large number and diversity, the prospect of redundancy, and the uncertainty regarding their individual contribution to NAFLD pathogenesis, especially across a heterogeneous patient population, all present challenges to translating this information into revealing pathophysiological mechanisms and informing therapeutic strategies. To help conceptualize this translational objective, we suggest that differentially expressed gene (DEG) signatures that map to differentially enriched pathways involved in the disease processes comprising the four NAFLD categories C₁-C₄ (see below and FIG. 1, Box E; Table 2; Methods) mirror emergent disease-specific networks (i.e., disease states) at different stages of disease progression. Pharmacologically normalizing these gene signatures using the integrative approach outlined in FIG. 1, Boxes E-L and as described herein is expected to modify disease progression in a clinically relevant human MPS model of NAFLD.

### Example 4

### Differentially Enriched Pathways Involved in Metabolic Dysfunction, Hepatic Inflammation, and Fibrosis.

The comparison of PLI vs. N&S, PF vs. N&S and PF vs. PLI pathway expression data yielded a total of 139 unique differentially enriched pathways (FDR<0.001) (Table S3; Data file S2). The distribution of the six KEGG pathway groups for each of the three pairwise comparisons are shown in FIG. 5A. Overall, this set of pathways is consistent with the intrinsic heterogeneity of MAFLD that reflects the diverse but convergent impacts of the environment, metabolism, comorbidities, and genetic risk factors (Friedman, Neuschwander-Tetri et al. 2018). More specifically, many of these differentially enriched pathways can be associated with at least one of four categories that comprise our current conceptual framework of MAFLD progression: C1) Insulin resistance and oxidative stress, C2) Cell stress, apoptosis and lipotoxicity, C3) Inflammation, C4) Fibrosis, (Sanyal 2019) as well as C5) Disease related pathways, C6) Other associated pathways that relate to comorbidities such as cardiovascular disease and cancer (FIG. 5B). Finally, a seventh category (C7) is comprised of three differentially enriched pathways with no clear association to NAFLD or the metabolic syndrome. The detailed pathway description and categorization can be found in Table S3 and Data file S2.

The 10 most differentially enriched pathways for both the PF versus N&S and the PLI versus N&S comparisons (FIG. 5C) are consistent with the metabolic underpinning, and the resultant cellular stress and inflammatory response intrinsic to MAFLD pathogenesis. In these two comparisons, dysregulated glyoxylate and dicarboxylate metabolism and fructose and mannose metabolism are evident. The former plays a major role in regulating TCA cycle anaplerotic flux, fatty acid synthesis versus degradation, gluconeogenesis and alpha ketoglutarate-dependent dioxygenases involved in epigenetic modifications of DNA, chromatin and the posttranslational modifications of MALFD-related transcription factors (Sinton, Hay et al. 2019). In the case of the latter, fructose uptake and metabolism is known to allosterically dysregulate and provide substrate for de novo lipogenesis (DNL) in MAFLD (Hannou, Haslam et al. 2018). Mannose itself has been associated with insulin resistance (Lee, Zhang et al. 2016) and its metabolism is critical for N-linked protein glycosylation and proteostasis. Dysregulation of amino sugar and nucleotide sugar metabolism that generates substrates for glycosyltransferases and glycosphingolipid biosynthesis in conjunction with reduced glycan degradation was also evident (FIG. 5C). Together, this multifactorial metabolic dysregulation of protein glycosylation and glycosphingolipid biosynthesis induces ER stress and a resultant unfolded protein response (UPR) as evidenced by both an increase in the ubiquitin-mediated proteolysis pathway and RNA degradation mediated in part by regulated IRE-dependent decay (RIDD)) (Lebeaupin, Vallee et al. 2018). The UPR promotes an inflammatory response as evidenced in part by an increase in the expression of the T cell receptor signaling pathway mediated by gap junction disruption and ultimately, an apoptotic response (FIG. 5C) (Lebeaupin, Vallee et al. 2018). Dysregulated and potentially uncoupled oxidative phosphorylation, lowered expression of the pentose phosphate pathway that compromises the reduction of glutathione to mitigate ROS, and an increase in the non-homologous end-joining to repair DNA oxidative damage in NAFLD (Nishida, Yada et al. 2016) indicate concurrent oxidative and ER stress promoted by dysregulated metabolism.

Complementary to the 10 most enriched pathways in each of the PF versus N&S and PLI versus N&S comparisons, the comparison between PLI and PF is consistent with fibrosis being the widely recognized hallmark of disease progression in NASH (FIG. 5C). Each of the 10 differentially enriched pathways in this latter comparison have been shown to have a role in hepatic fibrosis (Ramachandran and Iredale 2012, Zhao, Yu et al. 2017, Kennedy, Hargrove et al. 2018, Wang, Li et al. 2018, Zhu, Kim et al. 2018, Hintermann and Christen 2019, Sircana, Paschetta et al. 2019, Diedrich, Kummer et al. 2020) with several involved in hepatic stellate cell activation (Zhao, Yu et al. 2017, Wang, Li et al. 2018, Zhu, Kim et al. 2018). Unsurprisingly, the p-values of the top 10 differentially enriched pathways in the PF vs. N&S comparison were smaller than that of PLI vs. N&S comparison, indicating more significant changes of pathway dysregulation in later stage of MAFLD. In addition, the number of the top 10 differentially enriched pathways in the comparison PF vs. N&S playing a role in multiple disease categories was greater than the number in the PLI vs. N&S and PF vs. PLI comparisons indicative of enhanced disease complexity during progression. Details of the full list of differentially enriched pathways for each comparison can be found in Table S3 and Data file S2. Together, the analysis of this transcriptomic data set appears to have corroborated the clinical relevance of these differentially enriched pathways in the context of our conceptual framework of MAFLD progression (Friedman, Neuschwander-Tetri et al. 2018, Sanyal 2019).

Although each of these identified differentially enriched pathways has the potential to be a drug target, their large number and diversity, the prospect of redundancy, and the uncertainty regarding their individual contribution to MAFLD pathogenesis especially across a heterogeneous patient population, all present challenges to translating this information into therapeutic strategies. To help conceptualize this translational objective, we hypothesize that DEG signatures for each of the four NAFLD/MAFLD categories (FIG. 5; FIG. 18, Data file S2) will reflect emergent disease-specific networks at different stages of disease progression. These networks will be indicative of how individual category-specific differentially enriched pathways contribute and communicate with one another to form emergent disease-specific network hubs that can be pharmacologically modulated. We have tested this hypothesis using the integrative approach outlined in FIG. 1 E-J and below.

### Example 5

Connectivity Map (CMap) Analysis Predicts Drugs and Small Molecule Perturbagens Associated with MAFLD Progression

In order to predict drugs/small molecules that modulate individual components of MAFLD progression we focused on the DEGs that mapped to the categorized differentially enriched pathways (FIGS. 1D and 5C; Table S3 and Data file 51-2) identified in the three comparisons PLI vs. N&S, PF vs. N&S and PF vs. PLI. For each of these comparisons, a DEG signature resulted for each of the 4 NAFLD/MAFLD progression categories: C1. Insulin resistance and oxidative stress, C2. cell stress, apoptosis and lipotoxicity, C3. inflammation, C4. Fibrosis, generating a total of 12 gene signatures (Data file S3). Each of these 12 gene signatures was then used as input to query CMap (see (Subramanian, Narayan et al. 2017) and Methods).

CMap connects the differentially expressed gene signature between disease and non-disease states to drugs and other pharmacologically active compounds that can normalize the gene signature (Lamb, Crawford et al. 2006, Subramanian, Narayan et al. 2017, Keenan, Jenkins et al. 2018). In the context of this study, the output of CMap enables the pharmacologic testing of the hypothesis that normalization of the gene signatures between two disease states will halt or even reverse disease progression in an experimental human NAFLD model (see below). The output connectivity score ranges from −0.83 to 0.83 (see Data file S4), representing respectively the inverse to the most similar perturbation signature produced by the corresponding pharmacologic agent in comparison to the input signature. Since our initial objective is to identify drugs/small molecules that can be repurposed for preventing MAFLD progression, we focused on CMap outputs present in DrugBank (see Methods) that could promote the reversion of the disease-associated gene signature in each NAFLD category. The top 20 ranked drugs/small molecules for each of the 12 queries were selected, resulting in 139 unique predicted drugs/small molecules, 40 of which appeared as an output in more than one query (Table S5; Data file S4). Given the complex interplay among dysregulated metabolic pathways, oxidative and ER stress, inflammation and fibrosis during MAFLD progression, our initial prioritization of 20 drugs focused on those predicted to modulate multiple gene expression signatures (Table 1). Enriched in this set are drugs with targets known to be associated with MAFLD and with the potential to act pleiotropically to modulate several pathways. For example, isradipine, a dihydropyridine calcium channel blocker is predicted to modulate 5 signatures (Table 1). Calcium dyshomeostasis is induced in NAFLD by steatosis resulting in decreased Ca++ in the ER and increased Ca++ in both the cytoplasm and mitochondria. This imbalance further promotes steatosis, insulin resistance and ROS that can be reduced in human cell and murine models with calcium channel blockers that include dihydropyridines.

Likewise, the HSP90 inhibitor, geldanamycin, is predicted to modulate 4 signatures. One of the HSP90 client proteins involves the NLRP3 inflammasome, whose activation is considered to be a major contributor to liver inflammation and fibrosis in NASH. Accordingly, HSP90 inhibitors mechanistically indistinguishable from geldanamycin ameliorate NASH in murine models. The histone deacetylase inhibitor, vorinostat, and the JNK 1 inhibitor, AS061245 are predicted to modulate three signatures (Table 1) and each has targets associated with NAFLD. HDAC inhibitors have been associated with inhibition of stellate cell activation and liver fibrosis and JNK1 has a critical role in a feed forward activation loop amplifying the integrated deregulation among lipotoxicity, ER and oxidative stress and the NLRP3 inflammasome. In the context of these predictions and associations, it was surprising to observe the pure estrogen receptor antagonist, fulvestrant, appear in five signatures since estrogen itself is known to be protective against MAFLD. (Cheng, Wen et al. 2019, Bayoumi, Grønbæk et al. 2020).

### Example 6

Complementary Prioritization of Predicted Drugs from CMap Analysis Using Network Proximity

To prioritize the list of 139 drugs from CMap using an approach complementary to the one indicated in Table 1, we constructed a NAFLD subnetwork (FIG. 19; Methods) and used proximity to this network (Guney, Menche et al. 2016) to enhance the specificity and relevance of the CMap analysis for MAFLD. In essence, this algorithm connects NAFLD-associated gene signatures to drug-target profiles and maps the targets of a particular drug to the network protein nodes (FIG. 1, Boxes H-J; Methods). Drugs with target profiles that most closely overlap with a subset of protein nodes in the NAFLD network are prioritized for pharmacological testing in MAFLD models using human biomimetic liver MPS experimental models (FIG. 1, Box K and Methods). The current conceptual framework of NAFLD/MAFLD involves diverse but convergent pathways (Sanyal 2019, Eslam, Sanyal et al. 2020). The KEGG pathway database contains an annotated map of the stage-dependent progression of NAFLD (pathway id: hsa04932, (Kanehisa and Goto 2000, Kanehisa, Furumichi et al. 2017)). We used this NAFLD progression pathway as an anchor extending it with 10 interrelated pathways to generate a NAFLD subnetwork in the context of the liver protein-protein interactome (FIG. 19; Methods) From the total number of 9,904 DEGs (FDR p-value<0.001) in our three comparisons PLI vs. N&S, PF vs. N&S and PF vs. PLI, (Data file S1) 234 DEGs mapped to these 11 NAFLD associated pathways and the background liver PPI network (see FIG. 19; Methods). The degrees of the subnetwork nodes ranges from 0 to 64, with 9.7 neighbors on average for the 234 DEGs and ranges from 0 to 354, with 52.1 neighbors on average for the background liver network (Data file S6). Among the top 20 hub proteins (Table S6) were HSP90, -activated protein kinase 8 (MAPK8), NFκB essential modulator (IKBKG), protein kinase C alpha (PRKCA), caspase 8 (CASP8), signal transducer and activator of transcription 3 (STAT3), mitogen-activated protein kinase kinase kinase 7 (MAP3K7), and protein kinase C zeta type (PRKCZ). MAPK8 is a member of the MAP kinase and JNK family, acting as an integration point for multiple biochemicals signals, and is involved in 7 of the 11 NAFLD associated pathways including the NAFLD main pathway, TNF signaling pathway, Insulin signaling pathway, Type II diabetes mellitus, Adipocytokine signaling pathway, Protein processing in endoplasmic reticulum and Apoptosis. Among the 139 unique drugs/small molecules identified by our CMap analysis per se, 92 of these had targets in the liver background network (see Methods). These were then further evaluated by determining the network proximity between their targets and the NAFLD subnetwork (i.e., disease module) (FIG. 19; Methods), (Guney, Menche et al. 2016). The network proximity measure for each drug was represented by a z-score ranging from −3.7 to 1.6 (Data file S7). Negative z-scores indicate that the targets of the drug are more intrinsic to the disease module than a random set of targets. Therefore, the lower the z-score of a predicted drug the more likely it is to modulate the signaling in our NAFLD disease module. The 25 highest priority drugs and their known targets are shown in Table S7. As a result of the high degree of the HSP90 hub (Table S6), geldanamycin, highly ranked in the CMap analysis alone (Table 1) was the highest ranked drug from the network proximity analysis; it was accompanied by two other HSP90 inhibitors. The carboxylesterase 1 (CES1) inhibitor, TFA, also showed a high network proximity ranking resulting in part from its additional target, succinate dehydrogenase, the only enzyme participating in both the Krebs Cycle and the electron transport chain (i.e., Complex II). Genetic knockout of the corresponding CES1 gene, ces1d, in two independent NASH murine models lowered hepatic steatosis improved insulin sensitivity and protected mice from liver inflammation and fibrosis. The adenosine monophosphate-activated protein kinase (AMPK) agonist, phenformin also exhibited a high network proximity ranking. AMPK is reduced in NASH and agonists prevent NASH-induced hepatocyte apoptosis by enhancing AMPK mediated phosphorylation of procaspase-6 that in turn prevents its activation in murine NASH models. The thiazolidinedione, troglitazone, was also prioritized by network proximity. Though being highly ranked in only one signature-based query, troglitazone has many known targets in the MAFLD disease module (Table S5). Isradipine and AS-601245 two previously discussed drugs highly ranked by CMAp analysis that appeared in the queries from multiple signatures (Table 1) were also ranked highly in the network proximity analysis (Table S7).

### Example 7

### Experimental Testing of CMap Predicted Drugs in a Human Biomimetic Liver Microphysiological Model of MAFLD Progression

The human Liver Acinus MicroPhysiological System (LAMPS) is a platform containing primary human hepatocytes and liver sinusoidal endothelial cells (LSECs) as well as human Kupffer (THP-1) and stellate (LX-2) cell lines layered in the microfluidic device to partially recapitulate the structure and functions of the human liver acinus. We have recently demonstrated that this model system recapitulates key aspects of NAFLD progression including lipid accumulation, stellate cell activation, and the production of pro-inflammatory cytokines and fibrotic markers using media containing key MAFLD drivers including increased levels of glucose, insulin and free fatty acids. We also examined the effects of two control drugs that have shown marginal clinical benefit in NAFLD clinical trials, obeticholic acid (OCA) and pioglitazone (PGZ) as well as drugs identified by our computational analysis of MAFLD drug prediction (Table 1; Table S5 Data File S4, Data File S7) using the LAMPS experimental model (FIG. 17). LAMPS models were maintained for 10 days in MAFLD disease media containing either the indicated concentration of drug or DMSO vehicle control. We determined drug concentrations to test in LAMPS models guided by the CMap database and by cytotoxicity testing in primary hepatocytes (Table S8). In addition, we determined the amount of each compound that was adsorbed by the PDMS component of the LAMPS device (Table S8). Hepatocyte toxicity and drug adsorption were confounding parameters for some of the top-ranking drugs, so the testing of these were deprioritized for this study. We examined a panel of metrics to monitor disease-specific phenotypes including model functionality (albumin and blood urea nitrogen production) and cytotoxicity (lactate dehydrogenase secretion), hepatocellular steatosis (LipidTOX Lipid Stain from ThermoFisher Scientific labeling), stellate cell activation (α-smooth muscle actin staining), and the production of a panel of pro-inflammatory cytokines and fibrotic markers (Pro-collagen 1A1 and TIMP-1) (Mohammed et. al, 2021).

We first studied the two control drugs OCA and PGZ along with troglitazone (TGZ) that was predicted (Table S6). Although both PGZ and TGZ are members of the same drug class, glitazone members are known to exhibit distinct target profiles. Since in contrast to TGZ, PGZ was not highly ranked in our CMap analysis and since CMap does not bias against noncanonical targets, it was of potential interest to compare PGZ with TGZ in this model. LAMPS models were maintained for 10 days in NAFLD disease media containing 10 μM OCA, 30 μM PGZ, 10 μM TGZ or vehicle control (FIG. 7). Throughout the experimental time course, albumin, blood urea nitrogen and lactate dehydrogenase show no significant differences between vehicle control and drug treatment groups (FIG. 7A-C), suggesting no overt cytotoxicity and loss of function. However, there is a significant decrease in LipidTOX and α-SMA staining intensity in the OCA, TGZ and PGZ treatment groups compared to vehicle control demonstrating that both hepatocellular steatosis (FIG. 7D) and stellate cell activation (FIG. 7E) are reduced by treatment with these drugs. Although there is an ˜20% decrease in secretion of the pro-fibrotic marker Pro-Col 1a1 (FIG. 7F) with treatment of OCA, TGZ or PGZ, this decrease is not significant. In addition, there is also no significant change in the secreted levels of TIMP-1 in any of the treatment groups compared to vehicle (FIG. 7G).

We next examined the effect of two more compounds identified by our computational pipeline, the HDAC inhibitor, Vorinostat, and the JNK inhibitor AS601245. LAMPS models were maintained for 10 days in NAFLD disease media containing either Vorinostat (1.7 μM or 5 μM), AS601245 (1 μM or 3 μM), or DMSO vehicle control. As shown in FIG. 8, Vorinostat reduces stellate cell activation, the secretion of both pro-fibrotic markers and inflammatory cytokines in LAMPS models treated with NAFLD disease media. Albumin and blood urea nitrogen curves show no significant differences between vehicle and drug treatment groups (FIGS. 8A & B), suggesting that these drug treatments do not induce appreciable loss of hepatic functionality. However, there is a significant decrease in LDH secretion (FIG. 8C) at days 8 and 10 in the 5 μM Vorinostat treatment group, demonstrating that treatment with this drug alleviates disease media-induced cytotoxicity. This result is further supported by the overall significant decrease in the day 10 measurements of stellate cell activation (FIG. 8E; α-SMA intensity), production of the pro-fibrotic markers pro-collagen 1a1 and TIMP-1 (FIGS. 8F & G) and inflammatory cytokine production (FIG. 8H) observed in the vorinostat treatment group. In addition, within the AS601245 treatment group, we observed a general dose-dependent increase in the secretion of each of the cytokines assayed, suggesting that treatment with the higher dose of this compound resulted in an uptick of inflammatory signaling in the model (FIG. 8H). This is supported by the significant increase in day 10 LDH compared to the vehicle control for 3 uM AS601245 (FIG. 8C). Neither Vorinostat or AS601245 treatment alleviates lipid accumulation at day 10 compared to vehicle control (FIG. 8D), indicating no effect on steatosis. Overall, two of the predicted drugs used for the POC, TGZ and vorinostat, appear to exhibit complementary effects that mitigate NAFLD progression in the LAMPS, likely mirroring their distinct criteria for CMap drug prediction prioritization.

### Example 8

### Computational and Experimental Results

The liver is a highly complex organ composed of distinct cell types engaged in a wide range of functions that include macronutrient, amino acid and xenobiotic metabolism and glucose, lipid, and cholesterol homeostasis. In addition to its metabolic functions, the liver has several immunological roles, producing acute phase and complement proteins, cytokines and chemokines and is home to diverse populations of immune cells. Under normal environmental conditions, the liver is exposed to dietary and gut-derived pro-inflammatory bacterial products requiring a highly regulated integration of metabolic and immunological responses to preserve both tissue and organ homeostasis. This metabolic and immunologic regulatory network encompasses several conserved intra- and intercellular pathways and has evolved under the selection pressure of nutrient limitations. Current habits and lifestyles involving over-nutrition and the lack of physical activity impose challenges to this regulatory network in the form of vicious cycles of obesity-driven metabolic stress and aberrant immune responses leading to a chronic inflammatory state. Extensive pre-clinical and clinical studies have corroborated this dysregulated interplay between hepatic metabolism and immune responses and has provided a framework for understanding MAFLD progression. Nevertheless, this complex pathophysiology has thwarted therapeutic development as no drug to date has been approved for a NAFLD indication. To help address this unmet clinical need, we have implemented an integrated computational and experimental platform for predicting drug candidates from clinical data and demonstrating a proof of concept (POC) at this stage with a few of the predicted drugs in a human liver biomimetic MPS model of NAFLD progression.

Our present studies add significant knowledge to the current working model of MAFLD progression through the demonstration that distinct patterns of pathway expression, derived from RNA seq analysis of liver biopsies from a patient cohort encompassing the full spectrum of MAFLD progression, cluster with patients presenting with distinct clinical subtypes (i.e., simple steatosis, lobular inflammation, fibrosis) indicative of different stages of disease (See Methods). Accordingly, differentially expressed genes and pathways between any two of these three distinct stages were identified (FIG. 1, Box C & Box D; Methods). Each of these differentially expressed genes or pathways could represent a therapeutic target to halt or even reverse NAFLD progression. However, their large number and diversity, the prospect of redundant pathogenic mechanisms, and the uncertainty regarding their individual contribution to MAFLD pathogenesis across a heterogeneous patient population, all present challenges to translating this information into therapeutic strategies. Of fundamental importance, existing knowledge is not sufficient to determine how specific differentially expressed genes or associated pathways relate to emergent NAFLD-specific networks that likely involve complex dysregulated intra- and intercellular interactions (see above).

Given the daunting task of determining the mechanistic role single differentially expressed genes or even specific pathways play in the network pathophysiology of MAFLD, we implemented a mechanistically unbiased approach focused on molecularly phenotyping the progression of disease states (FIG. 1, Boxes E-G). We identified sets of genes differentially expressed between pairs of clustered samples each representing different NAFLD stages and sorted these gene sets into disease process categories (FIG. 1, Box E; Table S4 and Methods) according to our working NAFLD framework. These category specific gene signatures reflect changes in disease state at different stages of disease progression, but do not explicitly indicate a defined pathogenic (or compensatory) role in the context of a disease-specific network. We then implemented CMap analysis as part of this mechanistically unbiased approach to identify known drugs and other small molecule perturbagens that could invert these signatures and thereby normalize the disease state with the potential to halt or even reverse MAFLD progression (FIG. 1, Box F). Given the complex interplay among MAFLD associated processes and role of feedforward loops, we prioritized CMap predicted drugs based upon the frequency of appearances any one drug had across the 12 signatures (FIG. 1, Box G; Table S5). The highest priority drugs based on this criterion were enriched in those drugs with well-defined targets that would be expected to exhibit downstream pleiotropic modulation of cell processes that have been independently shown from the literature to be associated with NAFLD/MAFLD progression. Literature mining gave independent support for this drug prediction approach by providing evidence that modulation of targets of the predicted drugs by gene editing and/or drugs in the same mechanistic class showed benefit in murine models of NAFLD. Together these analyses support the mechanistically unbiased approach for predicting drugs that can mitigate MAFLD progression and provide the rationale for hypothesis testing in clinically relevant human biomimetic liver MPS experimental models.

We also implemented a complementary approach for prioritizing CMap predicted drugs using systems-informed network proximity as described in detail in the Methods and Results. We constructed a NAFLD subnetwork (FIG. 19; Methods) and used proximity to this network (Guney, Menche et al. 2016) to enhance the specificity and relevance of the CMap analysis for MAFLD. In essence, this algorithm connects NAFLD-associated gene signatures to drug-target profiles and maps the targets of a particular drug to the network protein nodes (FIG. 1, Boxes H-J; Methods). Drugs with target profiles that most closely overlap with a subset of protein nodes in the NAFLD network are prioritized. A limitation of this particular drug prioritization approach is the uncertainty of knowledge of the relative contribution of particular nodes to NAFLD progression. Despite this limitation, drugs such as geldanamycin and AS60125 whose targets HSP90 and JNK1 (MAPK8) are hubs with high degrees that would be expected to exhibit pleiotropic effects on the NAFLD-disease network were prioritized using either approach. In addition, drugs found in only one signature but having targets known to be associated with NAFLD such as troglitazone were also prioritized. Together, prioritization using these complementary approaches generated a diverse set of drugs for the present POC experiments, as well as detailed future testing singly and in combinations.

Even in the limited Proof of Concept (POC) experimental studies, we identified the HDAC inhibitor, vorinostat, that robustly reduced stellate cell activation and the secretion of both pro-fibrotic and inflammatory markers and significantly protected against disease-induced cell death during MAFLD progression in the LAMPS model without significant amelioration of hepatic steatosis. These experimental observations are consistent with vorinostat having a particularly high ranking in the output from the inflammation and fibrosis NAFLD category-specific CMap queries. In contrast, obeticholic acid and pioglitazone, selected as positive controls for the LAMPS studies on the basis of their marginal clinical benefit in NAFLD and troglitazone, identified using CMap analysis and prioritized through network proximity, all reduced steatosis and stellate activation but demonstrated no significant effect on secreted pro-fibrotic markers. The complementary beneficial effects of troglitazone and vorinostat in the LAMPS experimental model of MAFLD progression likely reflect in part their distinct selection criteria for CMap drug prediction prioritization and support the potential of complementary drug combinations as one therapeutic strategy.

The studies reported here support the use of the computational and experimental quantitative systems pharmacology platform described herein for identifying novel therapeutic strategies involving repurposed drugs for MAFLD. The present study focused on harnessing unbiased computational methods to predict drugs that might halt or even reverse the progression of MAFLD. However, we also selected a few of the predicted drugs to demonstrate a proof of concept (POC) for this approach using 2 doses of the drugs based on the CMax and published data where available.

The current studies have also identified two confounding factors for the POC experimental drug testing studies in the LAMPS-MAFLD disease model: 1) some predicted drugs induced toxicity at the literature-based doses used during a 10-day drug exposure; and 2) some drugs exhibited extensive (>75%) nonspecific drug adsorption to the PDMS components of the LAMPS experimental model making it risky to computationally correct the data. Therefore, we selected drugs that had little or no detected liver MPS toxicity over a 10-day drug exposure and little or no non-specific binding to the PDMS in the LAMPS model to demonstrate the POC.

### Example 9

### Creating a NAFLD Therapeutic Strategy

Recent advances in our human liver biomimetic MPS, the vascularized Liver Acinus MPS (vLAMPS), is the next stage MAFLD experimental model. The vLAMPS: 1) circumvents the large amount of the drug adsorbing polymer, PDMS, so that most drugs can be tested without significant corrections; 2) two channels connected by a 3 um pore filter allows the vascular channel to communicate with the hepatic channel that recapitulates the liver acinus structure allowing the delivery of drugs and immune cells under flow; 3) physiological continuous oxygen zonation is produced by controlling the flow in the two channels; and 4) the glass and plastic components allow real-time imaging to monitor temporal and spatial changes in a variety of metrics (Mhohammed et. al., 2021, Gough et. al., 2020) that extends the power of the metrics used to evaluate the response of the MAFLD experimental disease model to drugs and combinations.

We have initiated the detailed experimental investigation of the top 40-50 predicted drugs and combinations in the vLAMPS with full dose response curves using an expanded list of metrics including media efflux, a range of fluorescent protein biosensors and genomics. Instead of a mix of primary human liver cells (hepatocytes and LSECs) and well characterized human cell lines for stellate cells (LX2) and Kupffer cells (ThP-1), we are using all primary liver cells to build the MAFLD experimental vLAMPS model by isolating the cells from fresh liver resections where genomic characterization has been performed. These studies will serve as a reference data set for the future use of induced pluripotent stem cells (iPSCs) derived from individual patients that will have been characterized genomically and state of MAFLD disease (Gough et. al., 2020). The full power of human liver biomimetic MPS experimental models will be reached when we can analyze patient liver iPSC-derived cells to create patient cohorts based on genomic and disease state backgrounds.

The prospect for implementing iPSCs to make the vLAMPS (Gough et. al., 2020) patient-specific will help address the challenges of therapeutic development and clinical trial design imposed by patient heterogeneity intrinsic to MAFLD. However, in the near term, our POC studies suggest that combination strategies employing vorinostat and other predicted drugs that may modulate immune and fibrotic impacts and an approved anti-steatotic drug should be considered. In addition, the detailed drug and drug combination testing of the top predicted drugs identified in this study using all human primary cells in the vLAMPS MAFLD experimental disease model should lead to the identification of multiple drugs/combinations optimal for distinct patient cohorts in the near future.

### Example 10

### Initial Prediction and Testing of Drugs in a Human Liver MPS Model of NAFLD

To predict drugs/small molecules that modulate individual components of NAFLD progression, we initially focused on the DEGs (Data file S1) that mapped to the categorized (four NAFLD categories, C₁-C₄; Methods) differentially enriched pathways (FIG. 1, Boxes D-E; Data file S2; Methods) identified above in each of the 3 comparisons of unsupervised clusters (i.e., PLI vs. N&S, PF vs. N&S and PF vs. PLI) resulting in a total of 12 gene signatures (Table S4; Data file S3; Methods). Each of these 12 gene signatures was then used as input to perform connectivity mapping (CMap) on the LINCS database (see Subramanian et al., 2017 and Methods).

CMap connects the DEG signature between different disease states (including the non-disease state) to drugs and other pharmacologically active compounds predicted to normalize the disease-associated gene signature (see Methods)[Subramanian, A., et al. 2017, Keenan, A. B. et al. 2018, Lamb, J. et al. 2006]. In the context of this study, the output of CMap [Subramanian, A., et al. 2017, Keenan, A. B. et al. 2018, Lamb, J. et al. 2006] enables the pharmacologic testing of the hypothesis that normalization of the gene signatures between two disease states will halt or perhaps reverse disease progression in an experimental human NAFLD model (see below; Methods). Since a key objective is to identify drugs that can be repurposed for preventing NAFLD progression, we focused on CMap outputs present in DrugBank (see Methods) that could promote the reversion of the disease-associated gene signature in each NAFLD category (Methods; FIG. 1, Box F). For our initial study using the 2017 LINCS database, we selected the top 20 drugs (ranked by their most negative CMap score among all instances for that particular drug, see Methods) for each of the 12 queries, resulting in 106 unique predicted drugs, 35 of which appeared as an output in more than one query (FIG. 1, Box G; Table S5; Data file S4). Given the complex interplay among dysregulated metabolic pathways, oxidative and ER stress, inflammation, and fibrosis during NAFLD progression, our initial prioritization of 25 drugs focused on those predicted to modulate multiple gene expression signatures (FIG. 1, Box G; Table S5). Enriched in this set are drugs with targets known to be associated with NAFLD and with the potential to act pleiotropically, to modulate several pathways. For example, vorinostat is predicted to normalize 5 of the 12 signatures focused primarily on inflammation and fibrosis and previous studies in rodent models of NAFLD suggested efficacy with other HDAC inhibitors [Park, K. C., et al. 2014, Huang, H. M., et al. 2022].

We next used our LAMPS model of NAFLD to test the predicted drugs. The LAMPS model comprises an all-human cell platform containing primary hepatocytes and liver sinusoidal endothelial cells (LSECs) as well as Kupffer (differentiated THP-1) and stellate (LX-2) cell lines layered in a microfluidic device that recapitulates several key structural features and functions of the human liver acinus (FIG. 1, Box K, FIG. 17; Methods). The LAMPS model has been tested and reproduced by the Texas A&M Tissue Chip Validation Center (Tex-Val), one of the National Center for Advancing Translational Sciences (NCATS) funded Tissue Chip Testing Centers (TCTC). We have recently demonstrated that this model system recapitulates critical aspects of NAFLD progression including lipid accumulation, stellate cell activation, and the production of pro-inflammatory cytokines and fibrotic markers, using media containing key NAFLD drivers including increased levels of glucose, insulin and free fatty acids [Gough, A., et al. 2021] (FIG. 17; Methods). To gain further evidence supporting the clinical relevance of the LAMPS NAFLD model, we implemented a machine learning approach based on transcriptomic analysis of the 182 patient cohort [Gerhard, G. S., et al. 2018] described in FIG. 2 and Table S2 (Methods; FIG. 1, Box L). We first trained a multinomial logistic regression with elastic net penalization model (MLENet) using nested cross-validation to successfully differentiate among 4 clinical classifications of NAFLD (FIG. 31A). The final model used 71 genes with 80% of these having prior association with NAFLD. Using this patient-based model, we then classified the transcriptome of individual LAMPS under three media conditions, normal fasting (NF), early metabolic syndrome (EMS), and late metabolic syndrome (LMS) as shown in FIG. 1, Box L, FIG. 31B and the Methods. At the transcriptome level, progression of NAFLD in LAMPS upon media treatment mimics disease progression observed in patients, independently corroborating the biomarker and imaging data (FIG. 1, Box L, 31B).

We then examined the effects of two control drugs that have shown appreciable clinical benefit in NAFLD clinical trials, obeticholic acid (OCA) [Shah, R. A. and K. V. Kowdley 2020, Younossi, Z. M., et al. 2019] and pioglitazone (PGZ) [Musso, G., et al. 2017] using the LAMPS experimental model (FIG. 1, Box K, FIG. 32). LAMPS were maintained for 10 days in EMS media containing either the indicated concentration of drug or DMSO vehicle control. EMS conditions were selected since biomarker and imaging analysis indicate that steatosis, inflammation, and fibrosis are progressively induced during the 10-day testing period [Saydmohammed, M., et al. 2021]. We determined drug concentrations to test in LAMPS guided by the concentrations indicated in the LINCS L1000 database, reported PK/PD and by the absence of cytotoxicity at these concentrations during pre-testing in primary hepatocytes (Table S6). In addition, we determined the amount of each compound that was adsorbed by the PDMS component of the LAMPS device (Table S6). We examined a panel of metrics to monitor disease-specific phenotypes including model functionality (albumin and blood urea nitrogen production) and cytotoxicity (lactate dehydrogenase secretion), hepatocellular steatosis (LipidTOX® labeling), stellate cell activation (α-smooth muscle actin staining), and the production of a panel of pro-inflammatory cytokines (TNF-α, IL-6, IL-8, IL-1α and MCP-1) and fibrotic markers (Pro-collagen 1A1 and TIMP-1) [41] (FIG. 31B).

LAMPS models were maintained for 10 days in EMS media containing 10 μM OCA, 30 μM PGZ, or vehicle control (FIG. 32). Throughout the experimental time course, albumin, blood urea nitrogen and lactate dehydrogenase showed similar secretion profiles between vehicle control and drug treatment groups (FIG. 32A-C), suggesting no hepatocellular damage or loss of function. At the day 6 timepoint (FIG. 32A), there was a significant increase in albumin secretion in the OCA group; however, no further significant increases in albumin output were observed at later time points (days 8 and 10). However, there was a significant decrease in LipidTOX® and α-SMA staining intensity in the OCA and PGZ treatment groups compared to vehicle control demonstrating that both hepatocellular steatosis (FIG. 32D-E) and stellate cell activation (FIG. 32F-G) were reduced. Although there was a ˜20% decrease in secretion of the pro-fibrotic marker Pro-collagen 1a1 (FIG. 32H) with treatment of OCA, or PGZ, this decrease was not statistically significant, similar to other previous studies examining collagen 1 gene expression and secretion in response to treatment with OCA and PGZ [Leclercq, I. A., et al. 2006, Kostrzewski, T., et al. 2020]. In addition, there was also no significant change in the secreted levels of TIMP-1, another pro-fibrotic marker, in any of the treatment groups compared to vehicle (FIG. 32I).

We next examined the effect of the histone deacetylase (HDAC) inhibitor, vorinostat (abbreviated SAHA), the highest ranking drug predicted from our initial CMap analysis (FIG. 1, Box K, 32J-S; Table S5). LAMPS models maintained for 10 days in EMS disease media contained either vorinostat (1.7 μM or 5 μM), or DMSO vehicle control. As shown in FIG. 32, albumin and blood urea nitrogen curves showed no significant differences between vehicle and drug treatment groups (FIG. 32J-K), suggesting that these drug treatments do not induce appreciable loss of hepatic functionality. There was a significant decrease in LDH secretion (FIG. 32L) at days 8 and 10 in the 5 μM vorinostat treatment group, suggesting that treatment with this drug alleviates disease media-induced cytotoxicity. This result is further supported by the overall significant decrease in the day 10 measurements of stellate cell activation (FIG. 32O-P; α-SMA intensity), production of the pro-fibrotic markers pro-collagen 1a1 and TIMP-1 (FIG. 32Q-R) and inflammatory cytokine production (FIG. 32S) observed in the vorinostat treatment group. In contrast to PGZ and OCA, and despite its significant effect on profibrotic markers, vorinostat treatment did not appreciably alleviate lipid accumulation at day 10 (FIG. 32M-N), indicating no significant effect on steatosis.

Overall, the CMap predicted drug vorinostat in comparison to the control drugs PGZ and OCA, exhibited complementary effects that mitigated NAFLD progression in the LAMPS. To extend our initial proof-of-concept (PoC) findings, we tested LAMPS models maintained in EMS media containing either control or combinations of pioglitazone (30 μM) and vorinostat (1.7 μM or 5 μM) and monitored the same panel of disease-specific metrics. As shown in FIG. 33, while albumin secretion profiles showed no significant differences between vehicle and drug treatment groups, suggesting that these drug combinations did not result in loss of model functionality (FIG. 33A), a significant increase in urea nitrogen secretion was observed in both drug combination groups compared to control, suggesting increased model metabolic activity (FIG. 33B). In addition, like the LDH profile in FIG. 32, there was a significant decrease in LDH secretion (FIG. 33C) in the 5 μM vorinostat treatment group, suggesting a reduction in disease-induced cytotoxicity. In contrast to the individual drug testing studies shown in FIG. 32, we found an effect on the full complement of disease progression markers measured in this study when pioglitazone and vorinostat were used in combination, as we observed a significant reduction in both lipid accumulation (FIG. 33D-E) and stellate cell activation (FIG. 33F-G), as well as in the production of the pro-fibrotic markers pro-collagen.

### Expansion and Complementary Prioritization of CMap Predicted Drugs Using Network Proximity

During the course of these initial studies the LINCS L1000 database (accessible at clue.io) was significantly expanded providing an additional 1033 drugs that were annotated in DrugBank and accordingly, a more comprehensive set of perturbation instances that also encompassed additional cell lines. We took advantage of this larger biological representation by incorporating a percentile statistic for defining an overall CMap score for ranking drugs (FIG. 1, Boxes F-G; Methods and [Subramanian, A., et al. 2017]). Using this updated database, many drugs were identified ranking higher than vorinostat with the 25 highest shown in Table 6. Some of these drugs having canonical targets associated with NAFLD are predicted to revert 7 of the 12 cluster-based signatures. For example, the NSAID fenoprofen inhibits cyclooxygenase 1 and 2 to modulate prostaglandin synthesis and also activates the peroxisome proliferator receptors, alpha and gamma (PPARα/γ). The androgen receptor agonist oxandrolone, also predicted to revert 7 of the 12 signatures, promoted hepatic ketogenesis in an observational trial of adult males [Vega, G. L., et al. 2008] consistent with enhanced fatty acid partitioning from intrahepatic triglycerides towards mitochondrial beta oxidation and 4-hydroxybutyrate formation as proposed for the reversal of NAFLD resulting from a short-term ketogenic diet [Watanabe, M., et al. 2020, Luukkonen, P. K., et al. 2020]. Although several of the ranked drugs (Table 6) were structurally steroid-like, considerable structural diversity was evident in the predicted antibiotic and oncology drug classes. The cephalosporin, cefotaxime, interacts with the family of organic anion transporters (OATs or SLC22) whose expression is significantly altered during NAFLD progression [Li, T. T., et al. 2019]. These transporters mediate the hepatic disposition of drugs, xenobiotic metabolites and endogenous intermediates and metabolites. Targeting NAFLD associated hepatic proteins that have critical roles both in xenobiotic and endobiotic metabolism may be an emerging theme (see Example 11 and [Naik, A., et al. 2013]) that can be extended to nuclear receptor transcription factors as the diverse drugs tetracycline, SN-38, and the endogenous steroid, pregnanolone, have been shown to interact with PXR [Cave, M. C., et al. 2016, Sayaf, K., et al. 2021]. In a parallel CMap analysis based on queries derived from 12 patient subtype signatures (complementary to the set of 12 signatures derived from the unsupervised clusters, Table S4; Data files S3-5), 17/25 of the same predicted drugs (Table 6) were also identified and enriched in the highest ranked drugs.

As a complementary approach to prioritizing the 126 drugs from the CMap analysis (FIG. 1, Box G; Tables 6, S5) we constructed a NAFLD subnetwork (FIG. 1, Box H; Methods) and used proximity to this network [Guney, E., et al. 2016] as an approach to potentially enhance the specificity and relevance of the CMap analysis. In essence, this algorithm connects NAFLD-associated gene signatures to drug-target profiles and maps the targets of a particular drug to the network protein nodes (FIG. 1, Boxes H-J; Methods). Drugs with target profiles that most closely overlap with a subset of protein nodes in the NAFLD network are prioritized for pharmacological testing in our human liver biomimetic MPS experimental models (FIG. 1, Box K and Methods). The KEGG pathway database contains an annotated map of the stage-dependent progression of NAFLD (pathway id: hsa04932, [Kanehisa, M., et al. 2017, Kanehisa, M. and S. Goto 2000]). We used this NAFLD progression pathway as an anchor extending it with 10 interrelated pathways to generate a NAFLD subnetwork in the context of the liver protein-protein interactome (FIG. 1, Box H, Methods). From the total number of 9,904 DEGs (FDR p-value<0.001) in our three comparisons PLI vs. N&S, PF vs. N&S and PF vs. PLI, (Data file S1) 234 DEGs mapped to these 11 NAFLD associated pathways and the background liver PPI network (FIG. 1, Box H, Methods). The degrees of the subnetwork nodes range from 0 to 64, with 9.7 neighbors on average for the 234 DEGs and ranges from 0 to 354, with 52.1 neighbors on average for the background liver network (Data file S6). Among the top 20 hub proteins (Table S7; Data file S6) were HSP90, MAP kinase 8 (MAPK8), NFκB essential modulator (IKBKG), protein kinase C alpha (PRKCA), caspase 8 (CASP8), signal transducer and activator of transcription 3 (STAT3), mitogen-activated protein kinase kinase kinase 7 (MAP3K7), and protein kinase C zeta type (PRKCZ).

Among the 126 unique drugs identified by our CMap analysis per se, 45 had targets in the liver background network (see Methods). These were further evaluated by determining the network proximity between their targets and the NAFLD subnetwork (Methods) [Guney, E., et al. 2016]. The network proximity measure for each drug was represented by a z-score ranging from −2.8 to 2.1 (Data file S7; Methods). Negative z-scores indicate that the targets of the drug are more intrinsic to the disease module than a random set of targets. Therefore, the lower the z-score of a predicted drug the more likely it is to modulate the signaling in the NAFLD disease module. The 25 highest priority drugs and their known targets are shown in Table S7. Among the highest ranked drugs was fenoprofen, also highly ranked by signature frequency (Table 6) bolstering its prioritization for future testing. The HSP90 inhibitor, alvespimycin was also highly ranked by network proximity, consistent with HSP90 being a critical hub protein in the NAFLD subnetwork (Table S7; Data file S6). In addition, a closely related HSP90 inhibitor has been reported to modulate the activation of the NLRP3 inflammasome resulting in efficacy in murine models of NASH [Xu, G., et al. 2021]. A hallmark of NAFLD is hepatic calcium dyshomeostasis induced by steatosis that further promotes steatosis, insulin resistance and ROS that can be ameliorated in murine NASH models by the calcium channel blocker nifedipine [Nakagami, H., et al. 2012, Lee, S., et al. 2019]. Nifedipine and another calcium channel blocker, cinnarizine, were among the drugs ranked higher by network proximity. Two statins, fluvastatin and mevastatin were also identified by network proximity, consistent with recent meta-analyses [Doumas, M., et al. 2018, Lee, J. I., et al. 2021], suggesting the benefit of statin use in NASH development and progression.

### Example 11

An important outcome of the initial analysis in this study was the identification of differential pathway enrichment profiles among clinically defined stages of NAFLD progression. This information enabled disease states to be defined that could be targeted by systems-based approaches that are more comprehensive and less biased than traditional targeted approaches and therefore, may be better suited to address the heterogeneity and complex pathophysiology intrinsic to NAFLD. An unsupervised analysis of RNA-seq data from individual liver biopsies derived from a 182 NAFLD patient cohort encompassing a full spectrum of disease progression subtypes from simple steatosis to cirrhosis showed the presence of three patient clusters distinguishable by their pathway enrichment profiles and their predominant association with one of three clinical subtypes: normal/simple steatosis, lobular inflammation, or fibrosis. Pairwise comparisons among these clusters identified differentially enriched pathways consistent with the metabolic underpinning of NAFLD and the pathophysiological processes implicated in its progression that included lipotoxicity, insulin resistance, oxidative and cellular stress, apoptosis, inflammation, and fibrosis. The differentially enriched pathways identified among the pairwise comparisons of clusters originally derived from the unsupervised analysis showed significant congruence with those derived from the clinical subtypes within this patient cohort and through a meta-analysis, additional patient cohorts.

Guided by systems-based concepts and building upon the gene expression and pathway enrichment analyses, we implemented a QSP approach for defining NAFLD states, predicting drugs that target these states and testing the predicted drugs in human clinically relevant liver MPS NAFLD models. We defined disease states by first identifying differentially expressed genes for each of the pairwise comparisons among either the three unsupervised cluster groupings or among the three clinically defined clinical groups associated with disease progression. The differentially expressed genes that mapped to differentially enriched pathways were then categorized according to one (or more) of four categories of NAFLD pathophysiological processes in which the pathways are known to participate. This analysis resulted in two sets of twelve gene expression signatures reflecting different states of NAFLD progression. These signatures were then used to query the LINCS L1000 database to identify and rank drugs predicted to revert these gene signatures and accordingly, normalize their respective corresponding disease states [Subramanian, A., et al. 2017, Keenan, A. B., et al. 2017]. Among the higher CMap-ranked drugs two complementary criteria, frequency of appearance within each set of 12 signatures or NAFLD subnetwork proximity based on a predicted drug's known target profile were used for further prioritization for experimental testing.

To test the predicted drugs in a clinically relevant experimental system, we implemented a human liver acinus MPS, LAMPS, that recapitulates critical structural and functional features of the liver acinus [Lee-Montiel, F. T., et al. 2017, Vernetti, L. A., et al. 2016]. A large and diverse set of biomarkers and image-based analyses measured over time under different media that reflect normal fasting and early and late metabolic syndrome conditions, indicated that the human LAMPS also recapitulates critical aspects of NAFLD progression (e.g., simple steatosis, lipotoxicity, oxidative stress, insulin resistance, lobular inflammation, stellate cell activation and fibrosis) [Gough, A., et al. 2021, Saydmohammed, M., et al. 2021]. Nevertheless, with the translational goal in mind of identifying disease modifying therapies, it is important to know if these clinical phenotypes observed pre-clinically, arise through those mechanisms that occur in patients. To further establish the clinical relevance of LAMPS NAFLD model, we implemented a machine learning approach. We trained a transcriptome-based model from the 182 NAFLD cohort representing a full spectrum of disease progression subtypes to classify patients with high specificity. We then implemented this patient-based model consisting of 71 genes, with 57 of these having an independently determined association with NAFLD, to classify the transcriptomes of individual LAMP models treated under media conditions mirroring different stages of disease progression. The congruence between the patient-derived transcriptome-based classification of individual LAMPS and the diverse panel of NAFLD associated biomarker measurements supports the clinical relevance of the LAMPS as a NAFLD model. Two mechanistically distinct drugs, obeticholic acid and pioglitazone, that have shown some clinical benefit for NAFLD, were then tested as controls and both exhibited a hepatocellular antisteatotic effect and inhibition of stellate cell activation without an appreciable effect on profibrotic markers. We then tested the top ranked drug from an initial CMap analysis, the HDAC inhibitor vorinostat, predicted to primarily modulate inflammation and fibrosis. Consistent with the NAFLD CMap analysis and in contrast to the control drugs obeticholic acid and pioglitazone, vorinostat showed significant inhibition of proinflammatory and fibrotic biomarkers without an appreciable effect on steatosis. In addition, vorinostat ameliorated disease-induced cytotoxicity. Based on the complementary effects exhibited by vorinostat and the control drugs, the combination of vorinostat and pioglitazone was tested and demonstrated significant improvement across the full complement of NAFLD biomarkers. Altogether, these studies provide initial proof-of-concept for a patient-derived QSP platform that can infer disease states from gene expression signatures, predict drugs and drug combinations that can target these disease states and experimentally test these predictions in clinically relevant NAFLD models.

With the recent expansion of the LINCS L1000 database, we have identified several drugs predicted to be more efficacious than vorinostat for future testing and providing mechanistic inferences. Several of these predicted drugs have known interactions with proteins associated with NAFLD such as nuclear receptors, and bile and fatty acid transporters. In contrast, others had no known interactions with targets associated with NAFLD despite being predicted to reverse many of the same signatures. These drugs were either highly selective for a particular target such as topoisomerase (e.g., SN-38) or were antibiotics having minimum interactions with human proteins. Further analysis suggested a common thread among many of the predicted drugs that involve nuclear receptors such as PXR [Oladimeji, P. O. and T. Chen 2018] and the related constitutive androstane receptor. PXR is a transcriptional regulator capable of interacting with diverse exogenous and endogenous ligand modulators that has evolved in the liver to have xenobiotic/endobiotic metabolic functions in addition to functions regulating glucose/lipid metabolism/energy, inflammation, and stellate cell activation. Traditional targeted drug discovery approaches have identified FXR and PPAR agonists converging on this broader family of nuclear receptors intimately associated with NAFLD pathophysiology. The QSP approach described here has independently done so in a more comprehensive and unbiased manner with the potential to identify drugs/combinations more efficacious than obeticholic acid and pioglitazone by more completely targeting disease states. In essence, the systems-based platform described here can inform therapeutic strategies that are inherently more pleiotropic than traditional approaches and thus has the potential to address the complexity of transcriptional dysregulation intrinsic to diseases such as NAFLD [Yang, H., et al. 2021]. The finding that this can be achieved by repurposing approved drugs suggests that acceptable therapeutic indices could result by selectively modulating disease states. In conjunction with the advances in patient-derived iPSC technology [Collin de lHortet, A., et al. 2019] and in situ methods for RNA, metabolomic, and proteomic analyses, the QSP platform described in this study will become a mainstay for a personalized approach towards developing effective NAFLD therapeutic strategies.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method for quantitative systems pharmacology (QSP), comprising:
analyzing, using a computing device, RNA sequencing (RNA-seq) data for a plurality of patients having a disease, wherein the analysis identifies a plurality of differentially expressed genes (DEGs) and a plurality of differentially enriched biological pathways;
deriving, using the computing device, a plurality of gene expression signatures associated with each of a plurality of disease states of the disease using the DEGs and the differentially enriched biological pathways;
identifying, using the computing device, a plurality of drugs predicted to reverse a particular gene expression signature associated with a particular disease state of the disease; and
prioritizing for further experimental testing, using the computing device, the drugs predicted to normalize the particular gene expression signature and/or physiological characteristic associated with the particular disease state of the disease.

2. The method of claim 1, wherein the disease is metabolic dysfunction associated fatty liver disease (MAFLD) or non-alcoholic fatty liver disease (NAFLD).

3. The method of claim 2, wherein the disease states comprise entirely normal and steatosis, predominantly lobular inflammation, and predominantly fibrosis.

4. The method of claim 1, further comprising testing, using a microphysiological systems (MPS) platform, a drug or combination of drugs selected from the drugs predicted to normalize the particular gene expression signature and/or physiological characteristic associated with the particular disease state of the disease.

5. The method of claim 1, wherein the step of analyzing, using the computing device, the RNA-seq data comprises:
mapping a plurality of gene expression values to a plurality of biological pathway expression profiles; and
associating the biological pathway expression profiles with the disease states of the disease.

6. The method of claim 5, wherein the step of mapping the gene expression values to the biological pathway expression profiles comprises using a gene set variation analysis (GSVA) algorithm.

7. The method of claim 5, wherein the step of associating the biological pathway expression profiles with the disease states of the disease comprises using a clustering algorithm.

8. The method of claim 1, wherein the step of identifying, using the computing device, the drugs predicted to reverse the particular gene expression signature and/or physiological characteristic associated with the particular disease state of the disease comprises using a connectivity map (CMap).

9. The method of claim 1, wherein the step of prioritizing for further experimental testing, using the computing device, the drugs predicted to normalize the particular gene expression signature and/or physiological characteristic associated with the particular disease state of the disease comprises using a signature frequency ranking algorithm.

10. The method of claim 1, wherein the step of prioritizing for further experimental testing, using the computing device, the drugs predicted to normalize the particular gene expression signature and/or physiological characteristic associated with the particular disease state of the disease comprises using a network mapping algorithm.

11. The method of claim 10, wherein the network mapping algorithm considers best scores or percentile scores.

12. The method of claim 1, further comprising demonstrating, using a microphysiological systems (MPS) platform, that the drugs predicted to normalize the particular gene expression signature and/or physiological characteristic associated with the particular disease state of the disease reverses or halts the progression of the disease.

13. The method of claim 1, wherein the drugs predicted to normalize the particular gene expression signature and/or physiological characteristic associated with the particular disease state of the disease comprise a modulator directly acting on one or more targets with or without downstream pleiotropic effects to correct the particular disease state of the disease.

14. The method of claim 1, wherein the drugs comprise a compound defined by Formula I: 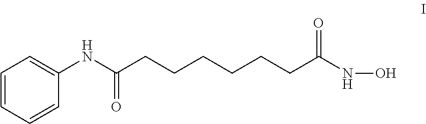 [SMILES: O=C(CCCCCCC(=O)Nc1ccccc1)NO]
or a pharmaceutically acceptable salt thereof.

15. The method of claim 14, wherein the drugs further comprise a compound defined by Formula II: 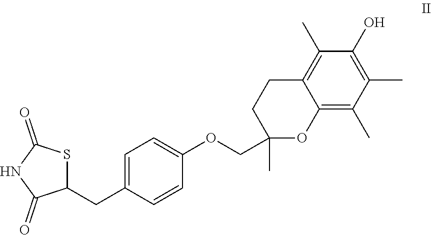 [SMILES: Cc1c(C)c2c(c(C)c1O)CCC(C)(COc1ccc(CC3SC(=O)NC3=O)cc1)O2]
or a pharmaceutically acceptable salt thereof.

16. The method of claim 1, wherein the drugs are selected from the group consisting of 7-hydroxystaurosporine, adenosine-phosphate, alfacalcidol cinnarizine, alvespimycin, alvocidib, ambrisentan, amorolfine, at-7519, auranofin, bezafibrate, brequinar, bromocriptine, brompheniramine, capsaicin, cebranopadol, cefotaxime, chlorpromazine, cladribine, curcumin, cytarabine, dasatinib, dexamethasone, dinoprost, dopamine, eltanolone, ephedrine, ethinylestradiol, fenofibrate, fenoprofen, fexaramine, fexofenadine, flucloxacillin, flucytosine, fluocinolone, Fluvastatin, fulvestrant, geldanamycin, gemcitabine, granisetron, gw-9662, hexestrol, indirubin, iohexol, isoprenaline, itraconazole, k-252a, medrysone, melphalan, mestranol, methylene-blue, mevastatin, midazolam, mifepristone, nifedipine, nitrendipine, norethindrone, Olaparib, olomoucine, oxacillin, oxandrolone, Palbociclib, pd-0325901, phenacetin, piceatannol, probenecid, proxyphylline, PX-12, pyrazolanthrone, ramipril, resveratrol, sildenafil, SN-38, streptozotocin, sulfanitran, tamoxifen, telmisartan, teniposide, tetracycline, thalidomide, trichostatin-a, troxerutin. Vemurafenib, vorinostat, wortmannin, derivatives thereof, and combinations thereof.

17. The method of claim 1, wherein the drugs are selected from the group consisting of 7-hydroxystaurosporine, adenosine-phosphate, alfacalcidol cinnarizine, alvespimycin, alvocidib, amorolfine, at-7519, auranofin, bezafibrate, brequinar, bromocriptine, brompheniramine, capsaicin, cebranopadol, cefotaxime, chlorpromazine, cladribine, curcumin, dasatinib, dexamethasone, dinoprost, dopamine, eltanolone, fenofibrate, fenoprofen, fexaramine, fexofenadine, flucloxacillin, fulvestrant, geldanamycin, gemcitabine, granisetron, gw-9662, hexestrol, iohexol, isoprenaline, itraconazole, k-252a, medrysone, melphalan, mestranol, methylene-blue, midazolam, nitrendipine, norethindrone, Olaparib, olomoucine, oxacillin, oxandrolone, Palbociclib, pd-0325901, phenacetin, piceatannol, probenecid, proxyphylline, PX-12, pyrazolanthrone, ramipril, resveratrol, sildenafil, SN-38, streptozotocin, sulfanitran, tamoxifen, telmisartan, teniposide, tetracycline, thalidomide, trichostatin-a, troxerutin, vorinostat, wortmannin, derivatives thereof, and combinations thereof.

18. The method of claim 1, wherein the drugs are selected from the group consisting of vorinostat, SN-38, auranofin, PX-12, methylene-blue, teniposide, trichostatin-a, trichostatin-a, dexamethasone, geldanamycin, capsaicin, curcumin, itraconazole, midazolam, Olaparib, chlorpromazine, fulvestrant, gemcitabine, alvocidib, brompheniramine, cladribine, dasatinib, dinoprost, fexaramine, fexofenadine, derivatives thereof, and combinations thereof.

19. The method of claim 1, wherein the drugs are selected from the group consisting of eltanolone, fenoprofen, oxandrolone, cefotaxime, amorolfine, dexamethasone, proxyphylline, sn-38, sulfanitran, tetracycline, 7-hydroxystaurosporine, dopamine, medrysone, mestranol, norethindrone, troxerutin, brequinar, bromocriptine, cebranopadol, flucloxacillin, granisetron, hexestrol, iohexol, melphalan, oxacillin, derivatives thereof, and combinations thereof.

20. The method of claim 1, wherein the drugs are selected from the group consisting of bezafibrate, geldanamycin, wortmannin, pd-0325901, piceatannol, fenofibrate, gw-9662, Palbociclib, alvespimycin, olomoucine, dasatinib, telmisartan, pyrazolanthrone, thalidomide, at-7519, nitrendipine, resveratrol, alvocidib, curcumin, probenecid, tamoxifen, sildenafil, methylene-blue, phenacetin, ramipril, derivatives thereof, and combinations thereof.

21. The method of claim 1, wherein the drugs are selected from the group consisting of isoprenaline, fenoprofen, streptozotocin, Palbociclib, 7-hydroxystaurosporine, alvespimycin, k-252a, adenosine-phosphate, alfacalcidol, cinnarizine, ambrisentan, hexestrol, nifedipine, mifepristone, Fluvastatin, mevastatin, cytarabine, ephedrine, ethinylestradiol, tetracycline, fluocinolone, indirubin, dopamine, flucytosine, vemurafenib, derivatives thereof, and combinations thereof.

22. The method of claim 1, wherein the drugs are selected from the group consisting of eltanolone, fenoprofen, oxandrolone, cefotaxime, amorolfine, dexamethasone, proxyphylline, sn-38, sulfanitran, tetracycline, 7-hydroxystaurosporine, dopamine, medrysone, mestranol, norethindrone, troxerutin, brequinar, bromocriptine, cebranopadol, flucloxacillin, granisetron, hexestrol, iohexol, melphalan, oxacillin, derivatives thereof, and combinations thereof.

23. The method of claim 1, further comprising analyzing the drugs predicted to normalize the particular gene expression signature and/or physiological characteristic associated with the particular disease state of the disease to identify a common thread for further experimental testing.

24. A method for treating non-alcoholic fatty liver disease (NAFLD) comprising administering the drugs identified by the method of claim 1 to a subject in need thereof in an effective amount to decrease or inhibit the disease.

25-33. (canceled)

34. A quantitative systems pharmacology (QSP) device, comprising:
at least one processor; and
a memory operably coupled to the at least one processor, wherein the memory has computer-executable instructions stored thereon that, when executed by the at least one processor, cause the at least one processor to:
analyze RNA sequencing (RNA-seq) data for a plurality of patients having a disease, wherein the analysis identifies a plurality of differentially expressed genes (DEGs) and a plurality of differentially enriched biological pathways;
derive a plurality of gene expression signatures associated with each of a plurality of disease states of the disease using the DEGs and the differentially enriched biological pathways;
identify a plurality of drugs predicted to reverse a particular gene expression signature associated with a particular disease state of the disease; and
prioritize for further experimental testing the drugs predicted to normalize the particular gene expression signature and/or physiological characteristic associated with the particular disease state of the disease.

35. The device of claim 34, wherein the step of analyzing the RNA-seq data comprises:
mapping a plurality of gene expression values to a plurality of biological pathway expression profiles; and
associating the biological pathway expression profiles with the disease states of the disease.

36. The device of claim 35, wherein the step of mapping the gene expression values to the biological pathway expression profiles comprises using a gene set variation analysis (GSVA) algorithm.

37. The device of claim 35, wherein the step of associating the biological pathway expression profiles with the disease states of the disease comprises using a clustering algorithm.

38. The device of claim 34, wherein the step of identifying the drugs predicted to reverse the particular gene expression signature and/or physiological characteristic associated with the particular disease state of the disease comprises using a connectivity map (CMap).

39. The device of claim 34, wherein the step of prioritizing for further experimental testing the drugs predicted to normalize the particular gene expression signature and/or physiological characteristic associated with the particular disease state of the disease comprises using a signature frequency ranking algorithm.

40. The device of claim 34, wherein the step of prioritizing for further experimental testing the drugs predicted to normalize the particular gene expression signature and/or physiological characteristic associated with the particular disease state of the disease comprises using a network mapping algorithm.

41. The device of claim 40, wherein the network mapping algorithm considers best scores or percentile scores.

42. The device of claim 34, wherein the memory has further computer-executable instructions stored thereon that, when executed by the at least one processor, cause the at least one processor to analyze the drugs predicted to normalize the particular gene expression signature and/or physiological characteristic associated with the particular disease state of the disease to identify a common thread for further experimental testing.

43. A quantitative systems pharmacology (QSP) system, comprising:
a microphysiological systems (MPS) platform; and
a computing device comprising at least one processor and a memory operably coupled to the at least one processor, wherein the memory has computer-executable instructions stored thereon that, when executed by the at least one processor, cause the at least one processor to:
analyze RNA sequencing (RNA-seq) data for a plurality of patients having a disease, wherein the analysis identifies a plurality of differentially expressed genes (DEGs) and a plurality of differentially enriched biological pathways;
derive a plurality of gene expression signatures associated with each of a plurality of disease states of the disease using the DEGs and the differentially enriched biological pathways;
identify a plurality of drugs predicted to reverse a particular gene expression signature associated with a particular disease state of the disease; and
prioritize for further experimental testing the drugs predicted to normalize the particular gene expression signature and/or physiological characteristic associated with the particular disease state of the disease, wherein the MPS platform is configured for testing a drug or combination of drugs selected from the drugs predicted to normalize the particular gene expression signature associated with the particular disease state of the disease.

44. The system of claim 43, wherein the step of analyzing the RNA-seq data comprises:
mapping a plurality of gene expression values to a plurality of biological pathway expression profiles; and
associating the biological pathway expression profiles with the disease states of the disease.

45. The system of claim 44, wherein the step of mapping the gene expression values to the biological pathway expression profiles comprises using a gene set variation analysis (GSVA) algorithm.

46. The system of claim 44, wherein the step of associating the biological pathway expression profiles with the disease states of the disease comprises using a clustering algorithm.

47. The system of claim 43, wherein the step of identifying the drugs predicted to reverse the particular gene expression signature and/or physiological characteristic associated with the particular disease state of the disease comprises using a connectivity map (CMap).

48. The system of claim 43, wherein the step of prioritizing for further experimental testing the drugs predicted to normalize the particular gene expression signature and/or physiological characteristic associated with the particular disease state of the disease comprises using a signature frequency ranking algorithm.

49. The system of claim 43, wherein the step of prioritizing for further experimental testing the drugs predicted to normalize the particular gene expression signature and/or physiological characteristic associated with the particular disease state of the disease comprises using a network mapping algorithm.

50. The system of claim 49, wherein the network mapping algorithm considers best scores or percentile scores.

51. The system of any-ene-f claim 43, wherein the memory has further computer-executable instructions stored thereon that, when executed by the at least one processor, cause the at least one processor to analyze the drugs predicted to normalize the particular gene expression signature and/or physiological characteristic associated with the particular disease state of the disease to identify a common thread for further experimental testing.
